SEQUENCE LISTING (32 sequences)

SEQ ID NO: 1 - DNA, 1910 bp, Homo sapiens
  modified_base (1)..(1910): “n” represents a, t, c, g, other or unknown
gtccttcgcgtcccggcggcgcggcggaggggccggcgtgacgcagcggttgctacgggccgcccttataaataaccgggctcaggagaaactttagcgagtcagagccgcgcacgggactgggaaggggacccacccgagggtccagccaccagccccctcactaatagcggccaccccggcagcggcggcagcagcagcagcgacgcagcggcgacagctcagagcagggaggccgcgcacctgcgggccggccggagcgggcagccccaggccccctccccgggcacccgcgttcatgcaacgcctggtggcctgggacccagcatgtctccccctgccgccgccgccgcctgcctttaaatccatggaagtggccaacttctactacgaggcggactgcttggctgctgcgtacggcggcaaggcggcccccgcggcgccccccgcggccagacccgggccgcgcccccccgccggcgagctgggcagcatcggcgaccacgagcgcgccatcgacttcagcccgtacctggagccgctgggcgcgccgcaggccccggcgcccgccacggccacggacaccttcgaggcggctccgcccgcgcccgcccccgcgcccgcctcctccgggcagcaccacgacttcctctccgacctcttctccgacgactacgggggcaagaactgcaagaagccggccgagtacggctacgtgagcctggggcgcctgggggctgccaagggcgcgctgcaccccggctgcttcgcgcccctgcacccaccgcccccgccgccgccgccgcccgccgagctcaaggcggagccgggcttcgagcccgcggactgcaagcggaaggaggaggccggggcgccgggcggcggcgcaggcatggcggcgggcttcccgtacgcgctgcgcgcttacctcggctaccaggcggtgccgagcggcagcagcgggagcctctccacgtcctcctcgtccagcccgcccggcacgccgagccccgctgacgccaaggcccccccgaccgcctgctacgcgggggccgggccggcgccctcgcaggtcaagagcaaggccaagaagaccgtggacaagcacagcgacgagtacaagatccggcgcgagcgcaacaacatcgccgtgcgcaagagccgcgacaaggccaagatgcgcaacctggagacgcagcacaaggtcctggagctcacggccgagaacgagcggctgcagaagaaggtggagcagctgtcgcgcgagctcagcaccctgcggaacttgttcaagcagctgcccgagcccctgctcgcctcctccggccactgctagcgcggcccccgcggcgtccccctggggccggccggggctgagactccggggagcgcccgcgcccgcgccctcgcccccncccccnnnnccgcaaaactttggcactggggcacttggcagcnggggagcccgtcggtaattttaatattttattatatatatatatctatattttgccaaccaaccgtacatgcagatggctcccgcccgtggtgtataaagaagaaatgtctatgtgtacagatgaatgataaactctctgctctccctctgcccctctccaggcccggcgggcggggccggtttcgaagttgatgcaatcggtttaaacatggctgaacgcgtgtgtacacgggactgacgcaacccacgtgtaactgtcagccgggccctgagtaatcgcttaaagatgttctagggcttgttgctgttgatgttttgttttgttttgttttttggtctttttttgtattataaaaaataatctatttctatgagaaaagaggcgtctgtatattttgggaatcttttccgtttcaagcaattaagaacacttttaataaacttttttttg

SEQ ID NO: 2 - DNA, 1038 bp, Homo sapiens
atgcaacgcctggtggcctgggacccagcatgtctccccctgccgccgccgccgcctgcctttaaatccatggaagtggccaacttctactacgaggcggactgcttggctgctgcgtacggcggcaaggcggcccccgcggcgccccccgcggccagacccgggccgcgcccccccgccggcgagctgggcagcatcggcgaccacgagcgcgccatcgacttcagcccgtacctggagccgctgggcgcgccgcaggccccggcgcccgccacggccacggacaccttcgaggcggctccgcccgcgcccgcccccgcgcccgcctcctccgggcagcaccacgacttcctctccgacctcttctccgacgactacgggggcaagaactgcaagaagccggccgagtacggctacgtgagcctggggcgcctgggggctgccaagggcgcgctgcaccccggctgcttcgcgcccctgcacccaccgcccccgccgccgccgccgcccgccgagctcaaggcggagccgggcttcgagcccgcggactgcaagcggaaggaggaggccggggcgccgggcggcggcgcaggcatggcggcgggcttcccgtacgcgctgcgcgcttacctcggctaccaggcggtgccgagcggcagcagcgggagcctctccacgtcctcctcgtccagcccgcccggcacgccgagccccgctgacgccaaggcccccccgaccgcctgctacgcgggggccgggccggcgccctcgcaggtcaagagcaaggccaagaagaccgtggacaagcacagcgacgagtacaagatccggcgcgagcgcaacaacatcgccgtgcgcaagagccgcgacaaggccaagatgcgcaacctggagacgcagcacaaggtcctggagctcacggccgagaacgagcggctgcagaagaaggtggagcagctgtcgcgcgagctcagcaccctgcggaacttgttcaagcagctgcccgagcccctgctcgcctcctccggccactgctag

SEQ ID NO: 3 - DNA, 969 bp, Homo sapiens
atggaagtggccaacttctactacgaggcggactgcttggctgctgcgtacggcggcaaggcggcccccgcggcgccccccgcggccagacccgggccgcgcccccccgccggcgagctgggcagcatcggcgaccacgagcgcgccatcgacttcagcccgtacctggagccgctgggcgcgccgcaggccccggcgcccgccacggccacggacaccttcgaggcggctccgcccgcgcccgcccccgcgcccgcctcctccgggcagcaccacgacttcctctccgacctcttctccgacgactacgggggcaagaactgcaagaagccggccgagtacggctacgtgagcctggggcgcctgggggctgccaagggcgcgctgcaccccggctgcttcgcgcccctgcacccaccgcccccgccgccgccgccgcccgccgagctcaaggcggagccgggcttcgagcccgcggactgcaagcggaaggaggaggccggggcgccgggcggcggcgcaggcatggcggcgggcttcccgtacgcgctgcgcgcttacctcggctaccaggcggtgccgagcggcagcagcgggagcctctccacgtcctcctcgtccagcccgcccggcacgccgagccccgctgacgccaaggcccccccgaccgcctgctacgcgggggccgggccggcgccctcgcaggtcaagagcaaggccaagaagaccgtggacaagcacagcgacgagtacaagatccggcgcgagcgcaacaacatcgccgtgcgcaagagccgcgacaaggccaagatgcgcaacctggagacgcagcacaaggtcctggagctcacggccgagaacgagcggctgcagaagaaggtggagcagctgtcgcgcgagctcagcaccctgcggaacttgttcaagcagctgcccgagcccctgctcgcctcctccggccactgctag

SEQ ID NO: 4 - DNA, 444 bp, Homo sapiens
atggcggcgggcttcccgtacgcgctgcgcgcttacctcggctaccaggcggtgccgagcggcagcagcgggagcctctccacgtcctcctcgtccagcccgcccggcacgccgagccccgctgacgccaaggcccccccgaccgcctgctacgcgggggccgggccggcgccctcgcaggtcaagagcaaggccaagaagaccgtggacaagcacagcgacgagtacaagatccggcgcgagcgcaacaacatcgccgtgcgcaagagccgcgacaaggccaagatgcgcaacctggagacgcagcacaaggtcctggagctcacggccgagaacgagcggctgcagaagaaggtggagcagctgtcgcgcgagctcagcaccctgcggaacttgttcaagcagctgcccgagcccctgctcgcctcctccggccactgctag

SEQ ID NO: 5 - protein, 345 aa, Homo sapiens
MQRLVAWDPACLPLPPPPPAFKSMEVANFYYEADCLAAAYGGKAAPAAPPAARPGPRPPAGELGSIGDHERAIDFSPYLEPLGAPQAPAPATATDTFEAAPPAPAPAPASSGQHHDFLSDLFSDDYGGKNCKKPAEYGYVSLGRLGAAKGALHPGCFAPLHPPPPPPPPPAELKAEPGFEPADCKRKEEAGAPGGGAGMAAGFPYALRAYLGYQAVPSGSSGSLSTSSSSSPPGTPSPADAKAPPTACYAGAGPAPSQVKSKAKKTVDKHSDEYKIRRERNNIAVRKSRDKAKMRNLETQHKVLELTAENERLQKKVEQLSRELSTLRNLFKQLPEPLLASSGHC

SEQ ID NO: 6 - protein, 322 aa, Homo sapiens
MEVANFYYEADCLAAAYGGKAAPAAPPAARPGPRPPAGELGSIGDHERAIDFSPYLEPLGAPQAPAPATATDTFEAAPPAPAPAPASSGQHHDFLSDLFSDDYGGKNCKKPAEYGYVSLGRLGAAKGALHPGCFAPLHPPPPPPPPPAELKAEPGFEPADCKRKEEAGAPGGGAGMAAGFPYALRAYLGYQAVPSGSSGSLSTSSSSSPPGTPSPADAKAPPTACYAGAGPAPSQVKSKAKKTVDKHSDEYKIRRERNNIAVRKSRDKAKMRNLETQHKVLELTAENERLQKKVEQLSRELSTLRNLFKQLPEPLLASSGHC

SEQ ID NO: 7 - protein, 147 aa, Homo sapiens
MAAGFPYALRAYLGYQAVPSGSSGSLSTSSSSSPPGTPSPADAKAPPTACYAGAGPAPSQVKSKAKKTVDKHSDEYKIRRERNNIAVRKSRDKAKMRNLETQHKVLELTAENERLQKKVEQLSRELSTLRNLFKQLPEPLLASSGHC

SEQ ID NO: 8 - DNA, 1500 bp, Mus sp.
gcccgttgccaggcgccgccttataaacctcccgctcggccgccgccgcgccgagtccgagccgcgcacgggaccgggacgcagcggagcccgcgggccccgcgttcatgcaccgcctgctggcctgggacgcagcatgcctcccgccgccgcccgccgcctttagacccatggaagtggccaacttctactacgagcccgactgcctggcctacggggccaaggcggcccgcgccgcgccgcgcgcccccgccgccgagccggccattggcgagcacgagcgcgccatcgacttcagcccctacctggagccgctcgcgcccgccgcggacttcgccgcgcccgcgcccgcgcaccacgacttcctctccgacctcttcgccgacgactacggcgccaagccgagcaagaagccggccgactacggttacgtgagcctcggccgcgcgggcgccaaggccgcgccgcccgcctgcttcccgccgccgcctcccgcggcgctcaaggcggagccgggcttcgaacccgcggactgcaagcgcgcggacgacgcgcccgccatggcggccggtttcccgttcgccctgcgcgcctacctgggctaccaggcgacgccgagcggcagcagcggcagcctgtccacgtcgtcgtcgtccagcccgcccggcacgccgagccccgccgacgccaaggccgcgcccgccgcctgcttcgcggggccgccggccgcgcccgccaaggccaaggccaagaagacggtggacaagctgagcgacgagtacaagatgcggcgcgagcgcaacaacatcgcggtgcgcaagagccgcgacaaggccaagatgcgcaacctggagacgcagcacaaggtgctggagctgacggcggagaacgagcggctgcagaagaaggtggagcagctgtcgcgagagctcagcaccctgcggaacttgttcaagcagctgcccgagccgctgctggcctcggcgggccactgctagcgcggcgcggtggcgtggggggcgccgcggccaccgtgcgccctgccccgcgcgctccggccccgcgcgcgcgcccggaccaccgtgcgtgccctgcgcgcacctgcacctgcaccgaggggacaccgcgggcacaccgcgggcacgcgcggcgcacgcacctgcacagcgcaccgggtttcgggacttgatgcaatccggatcaaacgtggctgagcgcgtgtggacacgggactacgcaacacacgtgtaactgtctagccgggccctgagtaatcaccttaaagatgttcctgcggggttgttgatgtttttggttttgtttttgttttttgttttgttttgtttttttttttggtcttattattttttttgtattatataaaaaagttctatttctatgagaaaagaggcgtatgtatatttgagaaccttttccgtttcgagcattaaagtgaagacattttaataaacttttttgggagaatgtttaaaagccaaa

SEQ ID NO: 9 - protein, 296 aa, Mus sp.
MHRLLAWDAACLPPPPAAFRPMEVANFYYEPDCLAYGAKAARAAPRAPAAEPAIGEHERAIDFSPYLEPLAPAADFAAPAPAHHDFLSDLFADDYGAKPSKKPADYGYVSLGRAGAKAAPPACFPPPPPAALKAEPGFEPADCKRADDAPAMAAGFPFALRAYLGYQATPSGSSGSLSTSSSSSPPGTPSPADAKAAPAACFAGPPAAPAKAKAKKTVDKLSDEYKMRRERNNIAVRKSRDKAKMRNLETQHKVLELTAENERLQKKVEQLSRELSTLRNLFKQLPEPLLASAGHC

SEQ ID NO: 10 - DNA, 1739 bp, Rattus sp.
aggggccccggcgtgacgcagcccgttgccaggcgccgccttataaacctccgctcggccgccgccgagccgagtccgagccgcgcacgggaccgggacgcagcggagcccgcgggccccgcgttcatgcaccgcctgctggcctgggacgcagcatgcctcccgccgccgcccgccgcctttagacccatggaagtggccaacttctactacgagcccgactgcctggcctacggggccaaggcggcccgcgccgcgccgcgcgcccccgccgccgagccggccatcggcgagcacgagcgcgccatcgacttcagcccctacctggagccgctcgcgcccgccgccgcggacttcgccgcgcccgcgcccgcgcaccacgacttcctttccgacctcttcgccgacgactacggcgccaagccgagcaagaagccgtccgactacggttacgtgagcctcggccgcgcgggcgccaaggccgcaccgcccgcctgcttcccgccgccgcctcccgccgcactcaaggccgagccgggcttcgaacccgcggactgcaagcgcgcggacgacgcgcccgccatggcggccggcttcccgttcgccctgcgcgcctacctgggctaccaggcgacgccgagcggcagcagcggcagcctgtccacgtcgtcgtcgtccagcccgcccgggacgccgagccccgccgacgccaaggccgcgcccgccgcctgcttcgcggggccgccggccgcgcccgccaaggccaaggccaagaaggcggtggacaagctgagcgacgagtacaagatgcggcgcgagcgcaacaacatcgcggtgcgcaagagccgcgacaaggccaagatgcgcaacctggagacgcagcacaaggtgctggagctgacggcggagaacgagcggctgcagaagaaggtggagcagctgtcgcgagagctcagcacgctgcggaacttgttcaagcagctgcccgagccgctgctggcctcggcgggtcactgctagcccggcgggggtggcgtgggggcgccgcggccaccctgggcaccgtgcgccctgccccgcgcgctccgtccccgcgcgcgcccgggcaccgtgcgtgcaccgcgcgcacctgcacctgcaccgaggggacaccgtgggcaccgcgcgcacgcacctgcaccgcgcaccgggtttcgggacttgatgcaatccggatcaaacgtggctgagcgcgtgtggacacgggactgacgcaacacacgtgtaactgtcagccgggccctgagtaatcacttaaagatgttcctgcggggttgttgctgttgatgtttttctttttgttttttgttttttgttttttttttggtcttattatttttttgtattatataaaaaagttctatttctatgagaaaagaggcgtatgtatattttgagaaccttttccgtttcgagcattaaagtgaagacattttaataaacttttttggagaatgtttaaaaaccttttgggggcagtagttggcttttgaaaaaaaattttttttcttccctcctgactttggatttatgcgagattttgttttttgtgtttctggtgtgtagggggctgcgggttatttttgggttgtgtgtggtggtgggtgggggtgtcgcatctgggtttttctcctcccctggcagatgggatgccagcccctccccccaggagagggggcagagtgccgggtcaggaattc

SEQ ID NO: 11 - DNA, 1390 bp, Rattus sp.
gaattccgggaccgggacgcagcggagcccgcgggccccgcgttcatgcaccgcctgctggcctgggacgcagcatgcctcccgccgccgcccgccgcctttagacccatggaagtggccaacttctactacgagcccgactgcctggcctacggggccaaggcgggccgcgccgcgccgcgcgcccccgccgccgagccggccatcggcgagcacgagcgcgccatcgacttcagcccctacctggagccgctcgcgcccgccgccgcggacttcgccgcgcccgcgcccgcgcaccacgacttcctttccgacctcttcgccgacgactacggcgccaagccgagcaagaagccgtccgactacggttacgtgagcctcggccgcgcgggcgccaaggccgcaccgcccgcctgcttcccgccgcctcccgccgcactcaagcccgagccgggcttcgaacccgcggactgcaagcgcgcggacgacgcgccgccatggcggccggcttcccgttcgccctgcgcgcctacctgggctaccaggcgacgccgagcggcagcagcggcagcctgtccacgtcgtcgtcgtccagcccgcccgggacgccgagccccgccgacgccaagcccgcccgccccctgcttcgcgggccgccggccgcgcccgccaagccaaggccaagaaggcggtggacaagctgagcgacgagtacaagatgcgagcgcaacaacatcgcggtgcgcaagagacaaggccaagatgcgcaacctggagacgcagcacaaggtgctggagctgacggcggagaacgagcggctgcagaagaaggtggagcagctgtcgcgagagctcagcacgctgcggaacttgttcaagcagctgcccgagccgctgctggcctcggcgggtcactgctaggccggcgggggtggcgtgggggcgccgcggccaccctgggcaccgtgcgccctgcccccgctccgtccccgcgcgcgcccggggcaccgtgcgtgcaccgggacctgcacctgcaccgaggggacaccgtcgcgcacgcacctgcaccgcgcaccgggtttcagggacttgatgcaatccggatcaaacgtggctgagcgcgtgtggacacgggactgacgcaacacacgtgtaactgtcagccgggccctgagtaatcacttaaagatgttctgcggggttgttgctgttgatgtttttgtttttgttttttgttttttgtttttttttggtcttattatttttttgtattatataaaaaagttctatttctatgagaaaagaggcgtatgtatattttgagaaccttttccgtttcgagcattaaagtgaagagcattttaataaacttttttggagaaaggaattc

SEQ ID NO: 12 - protein, 16 aa, Artificial Sequence
  Description of Artificial Sequence Synthetic Peptide
AAVALLPAVLLALLAP

SEQ ID NO: 13 - protein, 29 aa, Artificial Sequence
  Description of Artificial Sequence Synthetic Peptide
CFITKALGISYGRKKRRQRRRPPQGSQTH

SEQ ID NO: 14 - protein, 16 aa, Homo sapiens
MQRLVAWDPACLPLPP

SEQ ID NO: 15 - DNA, 105 bp, Artificial Sequence
  Description of Artificial Sequence Oligonucleotide
gatctgcagctggtaccatgggctaccatggaacgcctggtggcctgggacccagcatgctccccctgccgccgccgccgcctgcctttaaatccggagaagtgg

SEQ ID NO: 16 - DNA, 102 bp, Artificial Sequence
  Description of Artificial Sequence Oligonucleotide
ccacttctccggatttaaaggcaggcggcggcggcggcagggggagacatgctgggtcccaggccaccaggcgttccatggtagcccatggtaccagctgca

SEQ ID NO: 17 - DNA, 1042 bp, Homo sapiens
ctaccatggaacgcctggtggcctgggacccagcatgctccccctgccgccgccgccgcctgcctttaaatccggagaagtggccaacttctactacgaggcggactgcttggctgctgcgtacggcggcaaggcggcccccgcggcgccccccgcggccagacccgggccgcgcccccccgccggcgagctgggcagcatcggcgaccacgagcgcgccatcgacttcagcccgtacctggagccgctgggcgcgccgcaggccccggcgcccgccacggccacggacaccttcgaggcggctccgcccgcgcccgcccccgcgcccgcctcctccgggcagcaccacgacttcctctccgacctcttctccgacgactacgggggcaagaactgcaagaagccggccgagtacggctacgtgagcctggggcgcctgggggctgccaagggcgcgctgcaccccggctgcttcgcgcccctgcacccaccgcccccgccgccgccgccgcccgccgagctcaaggcggagccgggcttcgagcccgcggactgcaagcggaaggaggaggccggggcgccgggcggcggcgcaggcatggcggcgggcttcccgtacgcgctgcgcgcttacctcggctaccaggcggtgccgagcggcagcagcgggagcctctccacgtcctcctcgtccagcccgcccggcacgccgagccccgctgacgccaaggcccccccgaccgcctgctacgcgggggccgggccggcgccctcgcaggtcaagagcaaggccaagaagaccgtggacaagcacagcgacgagtacaagatccggcgcgagcgcaacaacatcgccgtgcgcaagagccgcgacaaggccaagatgcgcaacctggagacgcagcacaaggtcctggagctcacggccgagaacgagcggctgcagaagaaggtggagcagctgtcgcgcgagctcagcaccctgcggaacttgttcaagcagctgcccgagcccctgctcgcctcctccggccactgctag

SEQ ID NO: 18 - protein, 297 aa, Rattus sp.
MHRLLAWDAACLPPPPAAFRPMEVANFYYEPDCLAYGAKAGRAAPRAPAAEPAIGEHERAIDFSPYLEPLAPAAADFAAPAPAHHDFLSDLFADDYGAKPSKKPSDYGYVSLGRAGAKAAPPACFPPPPPAALKAEPGFEPADCKRADDAPAMAAGFPFALRAYLGYQATPSGSSGSLSTSSSSSPPGTPSPADAKAAPAACFAGPPAAPAKAKAKKAVDKLSDEYKMRRERNNIAVRKSRDKAKMRNLETQHKVLELTAENERLQKKVEQLSRELSTLRNLFKQLPEPLLASAGHC

SEQ ID NO: 19 - protein, 15 aa, Homo sapiens
KKTVDKHSDEYKIRR

SEQ ID NO: 20 - protein, 17 aa, Homo sapiens
RRERNNIAVRKSRDKAK

SEQ ID NO: 21 - DNA, 19 bp, Artificial Sequence
  Description of Artificial Sequence Primer
atgcaccgcctgctggcct

SEQ ID NO: 22 - DNA, 18 bp, Artificial Sequence
  Description of Artificial Sequence Primer
ctagcagtgacccgccga

SEQ ID NO: 23 - DNA, 30 bp, Artificial Sequence
  Description of Artificial Sequence Hybridization sequence
agcacgctgcggaacttgttcaagcagctg

SEQ ID NO: 24 - DNA, 30 bp, Artificial Sequence
  Description of Artificial Sequence Hybridization sequence
cagctgcttgaacaagttccgcagcgtgct

SEQ ID NO: 25 - DNA, 12 bp, Artificial Sequence
  Description of Artificial Sequence Synthetic oligonucleotide
gaattccatgca

SEQ ID NO: 26 - DNA, 10 bp, Artificial Sequence
  Description of Artificial Sequence Synthetic oligonucleotide
tagagtcgac

SEQ ID NO: 27 - DNA, 34 bp, Artificial Sequence
  Description of Artificial Sequence Synthetic oligonucleotide
agatctgcagctggtaccatggaattcgaagctt

SEQ ID NO: 28 - DNA, 33 bp, Artificial Sequence
  Description of Artificial Sequence Synthetic oligonucleotide
agatctgcagctggtaccatggaattccatgca

SEQ ID NO: 29 - DNA, 13 bp, Artificial Sequence
  Description of Artificial Sequence Synthetic oligonucleotide
atggaagtggcca

SEQ ID NO: 30 - DNA, 14 bp, Artificial Sequence
  Description of Artificial Sequence Synthetic oligonucleotide
tagagtcgaagctt

SEQ ID NO: 31 - DNA, 44 bp, Artificial Sequence
  Description of Artificial Sequence Synthetic oligonucleotide
gtgcagatccgagctcgagatctgcagctggtaccatggaattc

SEQ ID NO: 32 - DNA, 44 bp, Artificial Sequence; identical to SEQ ID NO: 31
  Description of Artificial Sequence Synthetic oligonucleotide

(19) United States
(12) Patent Application Publication
(10) Pub. No.: US 2003/0166586 A1
(43) Pub. Date: Sep. 4, 2003

(54) C/EBP beta isoforms and methods of use in cell regulation and anti-tumorigenesis

(76) Inventor: Linda Sealy, Brentwood, TN (US)

Correspondence Address: SHARI J CORIN PH.D, NEEDLE & ROSENBERG PC, THE CANDLER BUILDING, 127 PEACHTREE STREET NE, ATLANTA, GA, 30303-1811, US

(21) Appl. No.: 09/789,831

(22) Filed: Feb. 20, 2001

Related U.S. Application Data
(60) Provisional application No. 60/183,532, filed on Feb. 18, 2000.

(51) Int. Cl. A61K 48/00; A61K 39/395

(52) U.S. Cl. 514/44; 424/143.1

(57) ABSTRACT

The present invention relates generally to biological methods for treating tumors, including metastatic tumors. More particularly, the present invention discloses aberrant signal transduction pathways related to tumorigenesis. The present invention provides compositions and methods for treating tumors and improving the condition of a mammal in need thereof. The present invention further provides compositions and methods for regulation of cellular functions including, but not limited to: proliferation, differentiation, and cell death.

The present application claims right of priority under 37 U.S.C. §119(e) to the benifit of the earlier filing date for U.S. Provisional Application “C/EPBβ Isoforms and Methods of Use in Cell Regulation and Anti-Tumorigenesis”, Ser. No. 60/183,532, filed Feb. 18, 2000.

### BACKGROUND OF THE INVENTION

1. The present invention relates generally to compositions and methods for regulating cellular proliferation, differentiation, and death, including apoptosis. More particularly, the present invention provides compositions, methods, and kits related to the isoforms of C/EBPβ for treating tumors, including metastatic tumors, and inhibiting tumorigenesis including in a human or other mammal in need thereof. Also disclosed are compositions and methods related to C/EBPβ isoforms for maintaining a population of cells in a differentiated state, inhibiting proliferation of a population of cells, and promoting cell death in a population of cells.

2. State of the Prior Art

Tumors and Cancer

Normal cells reproduce and differentiate as the body requires. The proper amount of proliferation or the degree of differentiation varies according to each cell type, tissue type, the stage of development, and environmental conditions. Normal cells are under the control of the numerous regulatory factors, signals, and processes that help keep us healthy. If a cell becomes abnormal with regard to these regulatory systems, a tumor, or a abnormal mass of cells, can result. A benign tumor does not spread beyond its local environment and is life threatening only in certain cases. A malignant tumor is capable of spreading, or invading, other tissues and organs; even to distal parts of the body. The damage to cells, tissues, and organs that is caused by a malignant tumor is usually life threatening.

Standard Treatments for Tumors

Broadly, current methods of treatment for tumors include: surgery, radiation therapy, chemotherapy, hormone therapy, and biological therapy.

Highly localized benign tumors can be removed by surgical means. However, surgery is not usually effective for metastatic tumors because of the diffuse nature of such tumors. Also, surgery usually involves a level of pain and often requires the removal of a significant portion of normal tissue, for example, mastectomy.

Radiation therapy involves the destruction of cells in and around a tumor using high energy rays including radioactive particles. Radiation therapy is used to treat localized tumors of certain types, but the outcome is unpredictable on an individual basis. This unpredictability may result from radio-resistance of certain cells. Side effects of radiation therapy include weakness, nausea, loss of appetite, and a compromised immune system. Radiation therapy even could be counter-productive in apoptosis deficient tumors wherein the tumor cells do not die following radiation exposure, but the tumor fighting properties of the immune system are compromised.

Chemotherapy involves the use of drugs that kill rapidly dividing cells. The administration of chemotherapeutics is systemic usually so metastatic tumors can be treated. In addition to killing tumor cells, chemotherapeutics kill the cells in our bodies that should normally proliferate. Thus, chemotherapy is damaging to nearly every system of our bodies including the gastrointestinal tract, skin, hair, reproductive organs, and the immune system. Similar to radiation therapy, the outcome for chemotherapy is unpredictable on an individual basis; thus, chemotherapy could be counter productive if some of the tumor cells have a defective apoptotic machinery.

Hormone therapy is used to treat certain types of tumors that are hormonally dependent, such as, some breast and prostrate tumors. Hormone therapy typically involves the removal of hormone producing organs, such as the ovaries or the testicles; or the administration of hormone altering drugs and is effective in a small percentage of cases.

Biological Therapy for Tumors

Biological therapy entails the use of cells, genes, protein, and other “biological” devices and methods in the treatment of tumors. Most biological therapy has focused on the use of the immune system to treat tumors or to treat the side effects of other tumor therapies. Other biological therapies have focused on the use of genes and gene products that regulate cellular proliferation and apoptosis pathways. Biological therapy can be administered locally or systemically and, depending on the use, is more specific in affecting tumor cells that general irradiation, chemotherapy, or the like. Side effects of biological therapy vary with the treatment, but typically include flu like symptoms.

U.S. Pat. No. 5,545,563 to Darlington et al., describes a gene sequence that encodes the human CCAAT/enhancer binding protein (C/EBP), gene, and recombinant vectors that are capable of mediating the expression of the C/EBP gene. The C/EBP gene described in U.S. Pat. No. 5,545,563 currently is known as C/EBPα (for review of current nomenclature see, Lekstrom-Himes et al. J Biol Chem 273(44):28545-28548, 1998).

U.S. Pat. No. 5,532,220 to Lee et al., describes a method for utilizing p53 cDNA to suppress the neoplastic phenotype in mammalian cancer cells that are lacking normal p53.

U.S. Pat. No. 5,858,771 to Lee et al., describes the gene sequence that encodes the human retinoblastoma (Rb) gene and a vector containing the Rb gene. The specification describes a Rb gene and vector as being useful in gene therapy for suppressing the neoplastic phenotype.

U.S. Pat. No. 5,831,062 to Taylor et al., describes the human interferon consensus gene and use of this gene in eukaryotic expression systems. The specification describes methods for its use thereof in gene therapy, including the treatment of cancer.

The Need for Effective and Less Sever Treatment for Tumors

Fewer than 50% of people diagnosed with cancer in the United States survive five years. This five year all cancer types mortality rate for the United States has remained unchanged for at least one-hundred years. Progress has been made in treating some forms of cancer such as stomach; however, increases in mortality from other cancers, such as lung cancer, offset these gains. Breast cancer accounts for about 30% of all cancer deaths in women in the United States. Although breast cancer diagnosed in its earliest clinical stages (when confined to the breast) is highly curable with about a 97% five year survival, the survival rate for more advanced stages drops precipitously. When the breast cancer is at a regional stage (cancer has spread to surrounding tissues) the five year survival rate drops to about 76% and to about 20% when cancer is diagnosed at a distant stage (metastasized). Furthermore, young women with breast cancer (45 years or younger) tend to fare worse than older women.

Ovarian cancer also is relatively curable in its earliest stages, but the overwhelming majority of patients are diagnosed in stages III and IV. Although responsive to chemotherapy, most patients with advanced ovarian cancer relapse and die of their disease. Lung cancers also are typically diagnosed at a late stage wherein current treatment protocols are ineffective.

It is clear that effective treatment, especially with reduced side effects, is needed by patients with tumors and by physicians treating these disorders. In addition to the relief of suffering, such treatment will have tremendous commercial potential.

### SUMMARY OF THE INVENTION

The present invention overcomes deficiencies in the prior art including treating a tumor in a mammal in need thereof, inhibiting tumorigenesis, inhibiting tumor cell proliferation, inducing tumor cell differentiation, and promoting tumor cell death. The present invention also provides compositions and methods for the inhibition of tumorigenesis in a population of cells, maintaining a population of cells in a differentiated state, inhibiting proliferation of a population of cells, and promoting cell death in a population of cells.

In an unexpected and surprising series of discoveries, the inventor determined that the C/EBPβ-1, C/EBPβ-2, and C/EBPβ-3 isoforms of the CEBPB gene have distinct biological activities which are fundamental to the cellular balance between proliferation, differentiation, and cell death. The three known isoforms of C/EBPβ correspond to the first, second, and third in-frame AUG codons of the CEBPB gene in humans, mice, and rats. The inventor discovered biological activities and expression patterns of C/EBPβ-1, C/EBPβ-2, and C/EBPβ-3 that are characteristic of a tumor cell or a cell undergoing tumorigenesis. The present invention provides methods for treating a tumor in a mammal in need thereof, including using selective C/EBPβ isoforms for therapy.

The inventor discovered that C/EBPβ-1 promotes the differentiation of cells. The inventor further discovered that a characteristic of tumor cells is the loss of C/EBPβ-1 expression. Therefore, it is an aspect of the present invention that insertion (administration) and expression of a C/EBPβ-1 gene, or the insertion (administration) of a C/EBPβ-1 protein itself, into a tumor cell induces the tumor cell to differentiate and inhibits proliferation of the tumor cell. Furthermore, the insertion and expression of a C/EBPβ-1 gene, or the insertion of a C/EBPβ-1 protein itself, into a population of cells (including tumor cells and/or non-tumor cells) induces differentiation and inhibits proliferation of the population of cells.

The inventor discovered that C/EBPβ-2 promotes the proliferation of cells and that the aberrant expression of C/EBPβ-2 is related to tumorigenesis. Specifically, only tumor cells exhibit a nuclear expression of C/EBPβ-2. Therefore, it is an aspect of the present invention, that a characteristic of tumorigenesis, and tumor cells in general, is the gain of nuclear C/EBPβ-2 expression.

In anther aspect of the present invention, the inventor discovered that the insertion and expression of a C/EBPβ-3 gene, or the insertion of a C/EBβ-3 protein itself, into a tumor cell inhibits proliferation of the tumor cell and promotes cell death of the tumor cell. In addition, the insertion and expression of a C/EBPβ-3 gene, or the insertion of a C/EBPβ-3 protein itself, into a population of cells (including tumor cells and/or non-tumor cells) inhibits proliferation and promotes cell death of a population of cells.

It is an additional aspect of the present invention, that a portion of tumor cells or a portion of cells in a targeted population of cells will display the phenotype of beneficial effects of therapy by the method of the present invention even though the portion of cells does not actually acquire exogenous C/EBPβ-1 or C/EBPβ-3 during the course of therapy. This effect is known in the art with regard to treatment with other anti-tumor agents as the “bystander effect”. The phenotype of beneficial effects of therapy by the method of the present invention is meant to include, but not to be limited to: inhibition of tumorigenesis, inhibition of tumor cell proliferation, induction of tumor cell differentiation, and promotion of tumor cell death, the inhibition of tumorigenesis in a population of cells, the induction or maintenance of a population of cells in a differentiated state, inhibiting proliferation of a population of cells, and promoting cell death in a population of cells.

It is an object of the present invention to provide a method of treating a tumor in a mammal in need thereof, comprising administering an anti-tumor effective amount of selective C/EBPβ isoforms to a cell of the mammal. In certain preferred embodiments, the C/EBPβ isoform comprises a C/EBPβ-1 isoform, a C/EBPβ 3 isoform, or a combination of both C/EBPβ-1 and C/EBPβ-3 isoforms. In certain embodiments, the C/EBPβ-1 and/or C/EBPβ-3 isoforms are administered as proteins. In such embodiments, it is preferred that the administered formulation contain a minimal amount of a C/EBPβ-2 isoform of C/EBPβ and even more preferred that the formulation is essentially free of C/EBPβ-2. In certain still more preferred embodiments, the C/EBPβ-1 and/or C/EBPβ-3 isoforms are expressed from a nucleic acid which is introduced to the cell and which expresses the C/EBPβ-1 and/or C/EBPβ-3 isoforms. In such embodiments, it is preferred that the nucleic acid is incapable or made incapable of expressing a C/EBPβ-2 isoform. In certain exemplary embodiments, C/EBPβ-1 and C/EBPβ-3 (but not C/EBPβ-2) are expressed from a nucleic acid introduced to the cell.

It is an object of the present invention to administer the C/EBPβ isoforms as a protein or encoded in a nucleic acid(s) which expresses the C/EBPβ isoform in the cell of the mammal. It is preferred that nucleic acid constructs engineered to express the C/EBPβ-1 isoform be mutated to inhibit (and preferably prevent) the expression of the C/EBPβ-2 isoform, but that such mutation should not eliminate the therapeutic anti-tumor activity of the C/EBPβ-1 isoform.

It is an object of the present invention to provide a method of treating a tumor in a mammal in need thereof, including treatment of mammary tumors originating in a mammary epithelial cell.

It is an object of the present invention to provide a method of treating a metastatic tumor, wherein the metastatic tumor originated in a body component including, but not limited to the following: adipose, adrenal, bladder, bone, brain, central nervous system, cartilage, cervix, colon, endometrium, epidermis, epithelium, eye, fallopian tube, heart, intestine, joint, kidney, liver, lung, lymphoid, muscle, ovary, pancreas, peripheral nervous system, peritoneum, pluripotent stem cells (including lymphoid cells and myeloid cells), prostate, rectum, skin, spleen, stomach, tendon, testicle, uterus, and vasculature (e.g., vascular endothelium).

It is an object of the present invention to provide a method of treating a tumor, wherein the tumor is localized in a body component including, but not limited to the following: adipose, adrenal, bladder, bone, brain, central nervous system, cartilage, cervix, colon, endometrium, epidermis, epithelium, eye, fallopian tube, heart, intestine, joint, kidney, liver, lung, lymphoid, muscle, ovary, pancreas, peripheral nervous system, peritoneum, pluripotent stem cells (including lymphoid cells and myeloid cells), prostate, rectum, skin, spleen, stomach, tendon, testicle, uterus, and vasculature (e.g., vascular endothelium).

It is an object of the present invention to provide a method of treating a metastatic tumor, wherein the site of treatment is a body component including, but not limited to the following: adipose, adrenal, bladder, bone, brain, central nervous system, cartilage, cervix, colon, endometrium, epidermis, epithelium, eye, fallopian tube, heart, intestine, joint, kidney, liver, lung, lymphoid, muscle, ovary, pancreas, peripheral nervous system, peritoneum, pluripotent stem cells (including lymphoid cells and myeloid cells), prostate, rectum, skin, spleen, stomach, tendon, testicle, uterus, and vasculature (e.g., vascular endothelium).

It is an object of the present invention that administration of treatment includes, but is not limited to, the following modes and routes of administration: buccal, dermal, inhalation, injection, intradermal, intramuscular, intraocular, intraotic, intraperitoneal, intratumoral, intravenous, nasal, orthotopic, rectal, subcutaneous, topical, or vaginal.

It is an object of the present invention to provide a method of inhibiting angiogenesis within the tumor or disrupting the maintenance of the vessels supplying blood, oxygen, or nutrients to the tumor.

It is an object of the present invention to inhibit a proliferation of a population of cells in a mammal in need thereof.

It is an object of the present invention to maintain a state of differentiation in a population of cells in a mammal in need thereof.

It is an object of the present invention to promote cell death in a population of cells in a mammal in need thereof.

It is an object of the present invention to inhibit the formation of a tumor from a population of cells, and to inhibit the transformation of a tumor into a metastatic tumor, in a mammal in need thereof.

It is an object of the present invention to provide C/EBPβ isoforms combined with pharmaceutical preparations for treating a tumor in a mammal in need thereof.

It is an object of the present invention to provide kits, including pharmaceutically acceptable kits, for treating a tumor in a mammal in need thereof.

Accordingly, the present invention provides a method of treating a tumor in a mammal in need thereof, comprising administering an anti-tumor effective amount of an isolated C/EBPβ isoform to a cell of the mammal; wherein the C/EBPβ isoform is at least one of an isolated C/EBPβ-1 isoform or an isolated C/EBPβ-3 isoform. In certain embodiments, C/EBPβ-1 and C/EBPβ-3 are administered in their polypeptide forms. In certain embodiments, C/EBPβ-1 and/or C/EBPβ-3 is administered in the form of an isolated nucleic acid encoding C/EBPβ-1 and/or C/EBPβ-3 and which expresses C/EBPβ-1 and/or C/EBPβ-3 in the mammalian cell.

It is believed that any pharmaceutical formulation that is compatible with the delivery of proteins and/or nucleic acids can be utilized in certain embodiments of the present invention. A preferred pharmaceutical formulation comprises an isolated C/EBPβ-1 or an isolated C/EBPβ-3, or an isolated nucleic acid vector encoding a polypeptide thereof, mixed with a liposome. A combination of isolated C/EBPβ-1 and isolated C/EBPβ-3, or an isolated nucleic acid vector encoding C/EBPβ-1 and C/EBPβ-3 polypeptides, can also be mixed with a liposome for an effective pharmaceutical formulation.

It is preferred that treatment according to the present invention has at least one of the following effects on the tumor or the cells that comprise the tumor: inhibition of cell proliferation, induction of cell differentiation, shrinkage of the tumor mass, induction of cell death and inhibition of a secretion by the tumor. In certain embodiments, it is preferred that treatment inhibits the formation of blood vessels or inhibits the maintenance of blood vessels that supply the tumor with blood, oxygen, and nutrients. In certain embodiments, treatment is performed on cells that are not necessarily a part of a tumor, but wherein such treatment is detrimental to the existence of the tumor. Examples include, but are not limited to: treatment of vascular endothelial cells to inhibit angiogenesis, stimulation of the anti-tumorigenic potential of immune cells, the inhibition of tumor invasion into surrounding tissues through treatment of the cells surrounding the tumor, and the treatment of a population of cells to inhibit tumorigenesis.

Accordingly, a population of non-tumor cells can be treated with isolated C/EBPβ-1 to maintain the cells in a differentiated state and inhibit proliferation. A differentiated, non-proliferating state is detrimental to tumor formation; so, treating a population of non-tumor cells with isolated C/EBPβ-1 will impede tumorigenesis. Furthermore, a population of non-tumor cells can be treated with C/EBPβ-3 to inhibit proliferation and/or prime the cells for cell death. Inhibition of proliferation and induction of cell death are detrimental tumorigenesis; so, treating a population of non-tumor cells with isolated C/EBPβ-3 will impede or inhibit tumorigenesis. Also, cell death of tumor cells will cause the tumor to decrease in size, volume, or mass. In certain embodiments, isolated C/EBPβ-1 and C/EBPβ-3 are administered (polypeptide or expressed from an isolated nucleic acid). It is preferred that C/EBPβ-2 polypeptide is substantially removed or absent from the treating formulation. It is also preferred that C/EBPβ-2 expression is inhibited or eliminated in nucleic acid expression constructs for expressing C/EBPβ-1 and/or C/EBPβ-3.

Other objects, features and advantages of the present invention will become apparent from the following detailed description. It should be understood, however, that the detailed description and the specific examples, while indicating preferred embodiments of the invention, are given by way of illustration, since various changes and modifications within the spirit and scope of the invention will become apparent to those skilled in the art from this detailed description.

### BRIEF DESCRIPTION OF THE DRAWINGS

FIG. 1 is a diagram of C/EBPβ showing the relative sizes and positions of the polypeptide and polynucleotide forms of C/EBPβ-1, C/EBPβ-2, and C/EBPβ-3 isoforms of C/EBPβ and the recognition sites of the N-terminal peptide antibody and the C-terminal antibody (on a peptide sequence).

FIG. 2A is a diagram showing the National Center for Biotechnology Information (NCBI) LocusLink information for the C/EBPβ gene from human, mouse, and rat sources along with their respective gene products.

FIG. 2B is a diagram showing the LocusLink information for human C/EBPβ. As shown, the official gene symbol is “CEBPB” and the official gene name is “CCAAT/enhancer binding protein (C/EBP), beta”.

FIG. 2C is a diagram showing the LocusLink information for mouse C/EBPβ.

FIG. 2D is a diagram showing the LocusLink information for rat C/EBPβ.

FIG. 3A is a diagram of an annotated human C/EBPβ nucleotide sequence (SEQ ID NO:1).

FIG. 3B is a diagram of an annotated human C/EBPβ polypeptide sequence (SEQ ID NO:5).

FIG. 3C is a diagram of an annotated mouse C/EBPβ nucleotide sequence (SEQ ID NO:8).

FIG. 3D is a diagram of an annotated mouse C/EBPβ polypeptide sequence (SEQ ID NO:9).

FIG. 3E1-3 is a diagram showing one possible alignment of a rat C/EBPβ (also known as liver-enriched transcriptional activator protein or LAP) nucleotide sequence determined by U. Schibler ((Descombes et al. (1991) Cell 67:569-579) (SEQ ID NO:10) and the nucleotide sequence of a rat C/EBPβ clone identified by the present inventor in 1988. The boxes indicate the positions of sequence differences. The Schibler sequence has been withdrawn from GenBank database, but was obtained prior to its withdraw.

FIG. 3F is a diagram of an annotated rat C/EBPβ polypeptide sequence provided by the present inventor (SEQ ID NO:18). The underlined “G” (approximately position 41 in FIG. 3F) represents the glycine determined in the inventor's C/EBPβ clone (EF2, see FIG. 3E-1). The rat C/EBPβ polypeptide previously entered into the Entrez protein database, but now withdrawn, shows an alanine (A) at this position. In the mouse and human alignments to the rat sequence (FIGS. 5B, 5C), an alanine is found at this position. Glycine and alanine are a conservative residue substitution. It is possible that the difference is simply a polymorphism between clones.

FIG. 4A is a diagram of one possible alignment of the human and mouse C/EBPβ polypeptides (see FIGS. 3B and 3D). This particular alignment shows a 72.8% identity between the human and mouse C/EBPβ polypeptides shown.

FIG. 4B is a diagram of one possible alignment of the human and rat C/EBPβ polypeptides (see FIGS. 3B and 3F).

FIG. 4C is a diagram of one possible alignment of the mouse and rat C/EBPβ polypeptides (see FIGS. 3D and 3F). These polypeptides are about 99% homologous.

FIG. 5 is a diagram of an annotated human C/EBPβ-3 polypeptide showing predicted nuclear localization sequences KKTVDKHSDEYKIRR (underlined, SEQ ID NO:19) and RRERNNIAVRKARDKAK (double line overhead, SEQ ID NO:20).

FIG. 6A shows a Western blot of the expression of human C/EBPβ isoforms in human mammary epithelial cells (HMECs) and 7 cell lines derived from mammary tumors: MDA468, MDA 231, MCF7-neo, MCF7-218, MCF7, T47D, and BT474. Cytoplasmic and nuclear fractions were prepared from cultured HMECs and whole cell extracts were prepared from the mammary carcinoma derived cell lines.

FIG. 6B shows a Western blot of the expression of C/EBPβ-1, C/EBPβ-2, and C/EBPβ-3 isoforms in HMECs, normal breast epithelial cells from 3 different reduction mammoplasties, and from six different breast cancers. C/EBPβ-1 expression is confirmed by stripping and reprobing with a C/EBPβ-1 specific antibody.

FIG. 6C shows a Western blot of the expression of human C/EBPβ-1 isoform in human mammary epithelial cells (HMECs), milk ductal epithelial (MDE) cells and MCF-7 cells. C/EBPβ-1 was detected with the N-terminal C/EBPβ-1 specific antibody.

FIG. 6D shows a Western blot of C/EBPβ isoform expression in HMECs, MCF10A, and MCF7 cells. MCF7 is a model cell line for an aggressive breast carcinoma. MCF10 A is a model cell line for an intermediate stage of tumorigenesis of breast epithelial cells.

FIG. 6E shows a panel of four Western blots of C/EBPβ isoform expression assayed with a C-terminal antibody (top panels, recognizes all three C/EBPβ isoforms) and an N-terminal antibody (bottom panels, recognizes only the C/EBPβ-1 isoform). Ten tumor samples are assayed (left panels) and twelve normal samples are assayed (right panels).

FIG. 7 is a diagram showing the relative expression of C/EBPβ isoforms in HMECs, MDE (milk ductal epithelial) cells, primary tissues from breast reduction surgeries, primary tissues from surgically removed breast carcinomas, and cultured cells derived from breast carcinomas. FIG. 7 also shows the relative proliferation and differentiation status between each cell type.

FIG. 8A is a graph showing the relative luciferase activity generated by a luciferase reporter gene linked to approximately 1000 basepairs of the human cyclin D1 promoter in the presence of: a CMV driven expression vector for a human C/EBPβ-1 isoform or a CMV driven expression vector for a human C/EBPβ-2 isoform.

FIG. 8B shows two micrographs. The left micrograph shows a view using phase microscopy of a field of human mammary epithelial cells (HMECs) growing in cell culture. The right micrograph shows a view using fluorescent microscopy of the same field of HMECs stained with the C-terminal C/EBPβ antibody (which recognizes all three isoforms of C/EBPβ) and detected with Alexa 546 (red) conjugated secondary antibody. Punctate staining of C/EBPβ-2 was observed in the cytoplasm. Intense staining of C/EBPβ-1 and C/EBPβ-3 in the nucleus was also observed.

FIG. 8C is a diagram of a LZRS-his-C/EBPβ-IRES-eGFP vector. The vector allows expression of more than one gene from one transcript using the IRES technology (described herein). Specific vectors constructed include: a LZRS his-C/EBPβ-1 IRES-eGFP vector (preferably modified to prevent C/EBPβ-2 expression), a LZRS his-C/EBPβ-2-IRES-eGFP vector (modified to prevent C/EBPβ-3 expression in certain embodiments), and a LZRS-his- C/EBPβ-3-IRES-eGFP vector.

FIG. 8D (left panel) shows a Western blot of cyclin D1 expression in whole cell extracts of HMECs. Lane 1 is a sample of uninfected cells. Lane 2 is a sample of cells infected with LZRS IRES-eGFP. Lane 3 is a sample of cells infected with LZRS his- C/EBPβ-2 IRES-eGFP. These data show that C/EBPβ-2 expression stimulates cyclin D1 expression in HMECs. FIG. 8D (right panel) shows a Western blot of pRb (phosphorylated Rb) expression in whole cell extracts of HMECs. Lane 1 is a sample of uninfected cells. Lane 2 is a sample of cells infected with LZRS IRES-eGFP. Lane 3 is a sample of cells infected with LZRS his- C/EBPβ-2 IRES-eGFP. These data show that C/EBPβ-2 expression stimulates an increase in phosphorylation of Rb in HMECs (the band is shifted up indicating lower mobility in the gel).

FIG. 9A is a diagram showing the construction of the pRSETC-NFIL6 vector (also known as the pRSETC-C/EBPβ-1 vector) by linking the C/EBPβ-1 coding segment of the CMV-C/EBPβ-1 vector with the pRSETC vector. This adds a histidine tag to the C/EBPβ-1 sequence.

FIG.9B is a diagram showing the mutagenesis of pRSETC-C/EBPβ-1 by replacement of a portion of the vector with mutated oligonucleotides (SEQ ID NO:15 (top strand) and SEQ ID NO:16 (bottom strand))forming the pCDNA3.1HisA-C/EBPβ-1 vector. The C/EBPβ-2 translation start site (2nd in-frame ATG, see infra) is eliminated and a perfect Kozak sequence is created around the C/EBPβ-1 translation start site (1st in-frame ATG, see infra). This step prevents production of the C/EBPβ-2 isoform and enhances production of the C/EBPβ-1 isoform from the resulting insert when placed in an appropriate expression vector.

FIG. 9C is a diagram showing the transfer of the mutated polyhistidine tagged C/EBPβ-1 insert from the pCDNA3.1HisA-C/EBPβ-1 vector into the pLZRSpBMN-Z vector forming the pLZRShisC/EBPβ-1 vector. The pLZRSpBMN-Z vector is a hybrid retroviral/Epstein Barr expression vector (described in U.S. Pat. No. 5,830,725 to Nolan et al., incorporated herein by reference). The pLZRShisC/EBPβ-1 vector expresses C/EBPβ-1 in infected mammalian cells. The pLZRShisC/EBPβ-1 vector is not capable of expressing C/EBPβ-2.

FIG. 9D is a diagram of the pLZRShisC/EBPβ-1 vector.

FIG. 10A is a diagram showing the construction of the pcDNA3.1HisC/EBPβ-3 vector from prsetALip and pcDNA3.1HisC. The resultant vector adds a polyhistidine tag to the C/EBPβ-3 polynucleotide coding sequence.

FIG. 10B is a diagram showing the construction of the pLZRShisC/EBPβ-3 vector from the pcDNA3.1HisC/EBPβ-3 vector of FIG. 10A and the pLZRSpBMN-Z hybrid retroviral/Epstein Barr viral expression vector.

FIG.10C is a diagram of the pLZRShisC/EBPβ-3 vector.

FIG. 11 is a photo of two culture dishes of MDA 231 plated with 800 cells each. The cells in the left plate were infected with pLZRShisC/EBPβ-3 vector prior to plating. Colony formation was detected with hematoxylin stain.

FIG. 12 is a diagram of the preferred human DNA codons with the order of preference from left to right adjacent to each amino acid.

FIG. 13 shows three panels of MCF-10A mammary epithelial cells in culture that were infected with high titer pLZRS-his-C/EBPβ-2 retrovirus (2×10⁶ infectious units). The top left panel shows T7 tag staining by immunofluorescence microscopy with FITC conjugated (green) T7 tag antibody (indicating that all cells in the field are infected, also punctate staining of C/EBPβ-2 is observed in the cytoplasmic compartments of the cells). The top right panel shows DNA staining with BoPro3 (red, shows the nuclei). The bottom panel shows an overlay wherein essentially all nuclei contain yellow markings indicating a high level of C/EBPβ-2 expression.

FIG. 14 shows cellular foci formed when MCF-10A cells are infected with pLZRS-his-C/EBPβ-2 or LZRS-his-C/EBPβ-2-IRES-eGFP retroviral vector (separate experiments, shown in the three panels). No such foci were formed from LZRS IRES-eGFP retrovirus or from infection with LZRS his-C/EBPβ-1-IRES-eGFP retrovirus (data not shown). The formation of cellular foci is a model for transformation/tumorigenesis.

FIG. 15 shows MCF-10A cells infected with his C/EBPβ-2 retrovirus forming anchorage-independent growth colonies in soft agar. (left top and bottom panels), control MCF-10A cells which were not infected (middle top and bottom panels), and MDA 231 cells which are known to form anchorage-independent growth colonies in soft agar and to be tumorigenic when transplanted into animals (right top and bottom panels). Anchorage-independent growth colony formation is one model for tumorigenic characteristics of cultured cells.

FIG. 16 shows two micrographs of MDA 231 cells infected with LZRS-his-C/EBPβ-3-IRES-eGFP retrovirus. The left panel shows T7-tag antibody staining of C/EBPβ-3 (p20) expression) which is highly localized to the nucleus of these cells. The right panel shows green fluorescent protein (GFP) and T7-tag antibody detection in the same field of cells. All cells in the field express C/EBPβ-3.

FIG. 17 shows four micrographs of MDA 231 cells either infected with LZRS-his- C/EBPβ-3-IRES-eGFP (left top and bottom panels) or sham infected with LZRS-IRES-eGFP retrovirus (right top and bottom panels). The top panels (left and right) are of cells examined at day 10 post-infection. The bottom panels (left and right) are of cells examined at day 23 post infection.

FIG. 18 shows a quantitative FACS analysis of MDA 231 cells either infected with LZRS-his-C/EBPβ-3-IRES-eGFP (squares, solid line) or sham infected with LZRS-IRES-eGFP retrovirus (diamonds, dashed line) during a progression of days post-infection.

FIG. 19 shows a Western analysis of polypeptides in extracts from MDA 231 cells detected using the C-terminal C/EBPβ antibody. Lane 1 is an extract of cells sham infected with LZRS-IRES-eGFP retrovirus. Lane 2 is an extract of cells infected with LZRS-his-C/EBPβ-3-IRES-eGFP. Lane 3 is an extract of cells that were not infected. The relative positions are indicated for his-p20 (C/EBPβ-3) multimer, p42 (C/EBPβ-2), his-p20 (his- C/EBPβ-3), and p20 (C/EBPβ-3).

### DETAILED DESCRIPTION OF THE INVENTION

1.00 Definitions

The singular forms “a,” “an,” and “the” include plural references in this specification, including the claims, unless the content clearly dictates otherwise.

The present invention is not bound to theory or mechanism including the subject matter of the claims.

All U.S. Patents, foreign patents, patent disclosures, articles, books or sections of books, references, and citations provided herein are hereby incorporated in their entirety by reference and made part of this application.

In describing the present invention, the following terms are used. The meanings of the terms are understood by one of ordinary skill in the art and include the information provided below, which is listed by way of example so that the invention may be more easily understood.

As used herein, the terms “C/EBPβ-1” and “C/EBPβ-1 isoform” are used interchangeably. As used herein, the terms “C/EBPβ-2” and “C/EBPβ-2 isoform” are used interchangeably. As used herein, the terms “C/EBPβ-3” and “C/EBPβ-3 isoform” are used interchangeably. As used herein, the terms “C/EBPβ-1”, “C/EBPβ-2” and “C/EBPβ-3” are used to refer to three distinct isoforms of C/EBPβ (either protein or nucleic acid).

The terms “cancer” or “tumor” are clinically descriptive terms which are used to characterize cells or masses of cells that exhibit unchecked and abnormal cellular proliferation. The term “tumor”, when applied to tissue, generally refers to any abnormal tissue growth, i.e., excessive and abnormal cellular proliferation. A tumor may be “benign” and unable to spread from its original focus, or “malignant” and capable of spreading beyond its anatomical site to other areas throughout the host body. The term “cancer” is an older term which is generally used to describe a malignant tumor or the disease state arising therefrom. Alternatively, the art refers to an abnormal growth as a neoplasm, and to a malignant abnormal growth as a malignant neoplasm.

The terms “cell death” and “programmed cell death” are used herein interchangeably and are meant to include the process of “apoptosis” which is a well understood mechanism of programmed cell death in the art. The terms “cell death” and “programmed cell death” are specifically meant herein to include additional biological mechanisms through which a cell dies (ceases to function).

The term “transformation” has a number of meanings known to one with skill in the art. Initially one biological meaning to the term “transformation” was the genetic modification of a bacterium by incorporation of free DNA from another ruptured bacterial cell. The meaning of the term “transformation” has expanded to include the process through which a neoplasm becomes a malignant neoplasm and this meaning is included herein. The terms “transformation” and “tumorigenesis” are used interchangeably in certain embodiments herein. The formation of foci or anchorage-independent growth of colonies in soft agar are model systems for identifying “transformed” and “tumorigenic” cells. The term “tumorigenic” also means that the cells are capable of forming tumors in vivo when placed into an animal or transplanted from one animal to another.

As used herein, “isolated polynucleotide” means a polynucleotide the structure of which is not identical to that of any naturally occurring nucleic acid or to that of any fragment of a naturally occurring genomic nucleic acid spanning more than three separate genes naturally contiguous genes. The term therefore covers, for example, (a) a nucleic acid incorporated into a vector or into the genomic DNA of a prokaryote or eukaryote in a manner such that the resulting molecule is not identical to any naturally occurring vector or genomic DNA; (b) a separate molecule such as a cDNA, a genomic fragment, a fragment produced by polymerase chain reaction (PCR), or a restriction fragment; and (c) a recombinant nucleotide sequence that is part of a hybrid gene, i.e., a gene encoding a fusion protein. This definition of “isolated polynucleotide” supersedes and controls all other definitions known in the art.

As used herein, “hybridization probe” means a nucleic acid or mimetic that is labeled for detection, such as labeling with radiation (e.g., 33P, 32P, 14C, 3H labeled nucleotides), fluorescence, color, enzymatic detection, and the like. Labels and labeling systems or kits are readily available in the art. Hybridization probes (including nucleic acid mimetics, such as, peptide nucleic acids) are well known in the art.

As used herein, “culturing the cell” means providing culture conditions that are conducive to polypeptide expression. Such culturing conditions are well known in the art.

As used herein, “high stringency hybridization conditions” or “highly stringent hybridization conditions” means the following: hybridization at 42C in the presence of 50% formamide; a first wash at 65C with about 2×SSC containing 1% SDS; followed by a second wash at about 65C with 0.1×SSC.

The terms “nucleotide”, “nucleic acid”, “nucleic acid sequence”, “nucleotide sequence”, “DNA”, and “RNA” are known to one of ordinary skill in the art. Definitions of these terms are also found in the World Intellectual Property Organization (WIPO) Handbook on Industrial Property Information and Documentation, Standard ST.25: Standard for the Presentation of Nucleotide and Amino Acid Sequence Listings in Patent Applications (1998), including Tables 1 through 6 in Appendix 2, incorporated herein by reference. (Hereinafter “WIPO Standard ST.25 (1998)”). In certain embodiments of the present invention, the terms “nucleic acid”, “nucleic acid sequence”, “DNA”, and “RNA” include derivatives and biologically functional equivalents. In certain embodiments of the present invention, the terms “nucleic acid”, “nucleic acid sequence”, “polynucleotide” and “nucleotide sequence” are used interchangeably. These terms refer to a polymer of nucleotides of dinucleotide and greater, including polymers of 2 to about 100 nucleotides in length, including polymers of about 101 to about 1,000 nucleotides in length, including polymers of about 1,001 to about 10,000 nucleotides in length, and including polymers of more than 10,000 nucleotides in length.

The terms “amino acid” and “amino acid sequence” are known to one of ordinary skill in the art. Definitions of these terms are found in the WIPO Standard ST.25 (1998)″. In certain embodiments of the present invention, the terms “amino acid” and “amino acid sequence” include derivatives, mimetics, and analogues including D- and L-amino acids which may not be specifically defined in WIPO Standard ST.25 (1998). The terms “peptide”, “polypeptide”, and “amino acid sequence” are used interchangeably herein and refer to any polymer of amino acids (dipeptide or greater) typically linked through peptide bonds. The terms “peptide”, “polypeptide”, and “amino acid sequence” include oligopeptides, protein fragments, analogues, nuteins, and the like.

An “isolated” or “purified” polypeptide or polynucleotide as used herein refers to a polypeptide or polynucleotide that has been at least partially removed from its natural environment. An “isolated p20 polypeptide” is separated to some extent from the natural milieu of proteins and factors found in a mammalian cell expressing p20. In one example, a p20 polypeptide expressed in a host cell not of mammalian origin is an isolated p20 polypeptide. An “isolated p20 polynucleotide” does not include more than three contiguous genes (including the p20) as found in native genomic DNA.

The term “fusion protein” means a polypeptide sequence that is comprised of two or more polypeptide sequences linked by a peptide bond(s). “Fusion proteins” that do not occur in nature can be generated using recombinant DNA techniques. For example, a nucleic acid encoding a membrane transport sequence (membrane transport sequence) is part of an expression insert that also includes a nucleic acid sequence encoding p20. Expression of the insert results in the production of an MTS-p20 fusion protein. This could also be called a fusion polypeptide.

A “membrane transport signal” (also known as an “importation competent signal peptide”) is a sequence of amino acids generally of a length of about 10 (possibly fewer) to about 50 or more amino acid residues, many (typically about 55-60%) residues of which are hydrophobic such that they have a hydrophobic, lipid-soluble portion. The hydrophobic portion is a common, major motif of the signal peptide, and it is often a central part of the signal peptide of protein secreted from cells. The MTS peptides of this invention are “importation competent,” i.e., capable of penetrating through the cell membrane from outside the cell to the interior of the cell. Specific MTS sequences are provided in U.S. Pat. No. 6,043,339 to Lin et al. and U.S. Pat. No. 5,807,746 to Lin et al., each patent incorporated herein by reference.

In certain embodiments, the terms “peptide”, “polypeptide”, “protein”, and “amino acid sequence” are used interchangeably and refer to any polymer of amino acids (dipeptide or greater) linked through peptide bonds. In some instances, the term “peptide” is used to indicate a chain of amino acids that is a dinucleotide or greater. The term “peptide” is often used in the art to refer to an amino acid chain of about 2 to about 100 residues in length, but sometimes longer. In some instances the term “polypeptide” is used to refer to a chain of amino acids that is about 100 residues in length or more. In some instances, the term “protein” is used to refer to a polymer of amino acids of any length that is folded in a biologically appropriate conformation conducive to the activity of the “protein” (which may include multiple conformations). As used herein, the terms “peptide”, “polypeptide”, “protein”, and “amino acid sequence” are used interchangeably to mean an amino acid chain of about 2 residues or longer, including: 2 residues to about 100 residues, from about 101 residues to about 1,000 residues, from about 1,001 residues to about 10,000 residues, and more than 10,000 residues. As used herein, the terms “peptide”, “polypeptide”, “protein”, and “amino acid sequence” include oligopeptides, protein fragments, analogues, nuteins, fusion proteins and the like.

Meanings of the term “gene expression” are known to those with skill in the art. “Gene expression” includes the production of a protein from RNA or DNA and production of a RNA from a DNA. A gene is said to be “expressed” when it is transcribed into RNA, but this meaning also includes translation into a peptide or protein. The term “gene expression” is often shortened to “expression”, “expressed”, or the like. Additional meanings of the term “gene expression” are known to those with skill in the art.

The terms “transfect”, “transfection” or “transfecting” are used to indicate the act or method of introducing a molecule, usually a nucleic acid, into a cell.

The meanings of the terms “differentiated”, “differentiated cell”, and “differentiation” include herein the state of the cell in which it can perform certain specialized functions not common to all cells of the organism.

The meanings of the term “consensus sequence” include herein a sequence of general agreement between multiple nucleic acid or peptide sequences that are aligned and examined for sequence similarities. Several programs are commonly used in the scientific community to perform sequence alignments. These include BLAST for nucleotide sequence alignments and FASTA for peptide or protein alignments. In certain embodiments herein, the term “consensus sequence” refers to a window of similarity which may or may not include the entire sequence of one or more sequences under comparison.

The terms “homologous”, “homology”, “sequence homology” can be used interchangeably and indicate a relative degree of sequence identity between two or more biologically relevant sequences. Homology can be determined, for example, between two peptide sequences by aligning the sequences to obtain a best alignment or a preferred alignment (programs such as FASTA in the case of peptide sequences can be helpful); the number of identical amino acids in the alignment and the total number of amino acids are counted; and the homology is usually represented as a percentage (the ratio of identical units to total units (amino acids in this example) multiplied by one-hundred). In certain embodiments of the present invention, the homology of a derivative sequence is determined for some or all of the derivative sequence and for some or all of the comparison sequence. In certain embodiments of the present invention, the homology of a biological equivalent sequence is determined for some or all of the biologically equivalent sequence and for some or all of the comparison sequence.

The terms “treating” and “therapy” mean the reduction or elimination of symptoms of a disease of interest. This can be through alteration of physiological or molecular level abnormalities. Therapy can also refer, herein, to the reduction or elimination of signs, symptoms, or conditions of disease through unknown mechanism as the present invention is not bound by theory or mechanism. The term “treatments” can also refer to a substance or process applied to an experimental or medical condition.

Meanings of the terms used herein are known to those of ordinary skill in the art and can include meanings not specifically mentioned in the definitions above which are provided by way of example. Other terms and meanings are provided herein.

2.00 Proliferation, Differentiation, Cell Death, and Tumorigenesis

There is persuasive evidence that abnormal cellular proliferation and differentiation is the result of progressive failures of multiple genetic and tissue mediated regulatory mechanisms. These mechanisms are thought to act in the nucleus, cytoplasm, at cellular membranes, and in the tissue-specific environment of the cell. The process of transformation of a cell from a normal state to a condition of excessive or abnormal cellular proliferation is called tumorigenesis. Tumorigenesis can also include an abnormal cellular differentiation state or a defective ability of a cell to undergo cell death including apoptosis.

Tumorigenesis is considered a multistep progression from a normal cellular state to, in some instances, a full malignancy because the biological activity of multiple regulatory systems must be degraded for progression through tumorigenesis to occur. It is widely believed that multiple genetic mutations (commonly referred to as “hits”) of the cellular regulatory mechanisms result in a series of cumulative tumorigenic events including a clonal selection of cells with increasingly aggressive growth properties, defective abilities to differentiate, and defective abilities to undergo cell death. The literature is rife with examples of defects in proliferation, differentiation, and cell death pathways being associated with tumorigenesis.

2.10 Molecular Defects and Mutations in Tumorigenesis

At least four important classes of genes: protooncogenes, tumor suppressor genes, DNA repair genes, and cell cycle related genes are thought to be targeted by mutations as related to tumorigenesis. Certain mutations in protooncogenes such as c-ras, c-fos, and c-myc have been observed to promote tumorigenesis. The protooncogenes are involved in cellular proliferation signaling pathways which are normally switched off in non-dividing cells, but protooncogene mutation is believed to result in a gain of mitogenic function and a step toward tumorigenesis.

Germline mutations in the tumor suppressor genes p53, Rb, and p16 are associated with inherited cancer syndromes. It is widely held that tumor suppressors act as cellular brakes to proliferation in abnormal environments. If cells with a normal complement of tumor suppressor factors are inappropriately stimulated to divide, the tumor suppressors induce apoptosis which interrupts the tumorigenic process. Thus, defects in these and other tumor suppressers are associated with tumorigenesis.

Many of the tumor suppressors act on cell cycle related factors to inhibit cell division at this fundamental level. The mutation of a cell cycle factor such that it can no longer interact with a tumor suppressor also is known to promote tumorigenesis. Alternatively, defects leading to the overexpression of cell cycle factors can force the cell through the proliferative cycle. In one such example, cyclin D1 expression is observed in about 50% of all breast carcinomas. Furthermore, the overexpression of cyclin D1 in transgenic mice results in the development of mammary carcinomas.

Defects that result in over activity of numerous growth factors is also associated with tumor development. For example, certain tumors overexpress epidermal growth factor receptor (EGFR), erbB-2 (also known as HER2 and neu), the Ras-Raf-MAPK pathway, c-fos, c-jun, c-myc, and c-max. This list is not limiting. Also, defects in DNA repair genes are known to underlie certain syndromes associated with multiple and/or metastatic tumor formation.

2.20 Mammary Gland Development and Breast Cancer

The human mammary gland undergoes a remarkable course of postnatal development that can be divided into four stages. Ductal outgrowth and branching into the stromal fat pad occurs at puberty, lobuloalveolar proliferation and differentiation accompanies pregnancy, synthesis and secretion of milk by functionally differentiated secretory epithelial cells occurs during lactation, and at involution the entire alveolar epithelial compartment is remodeled to resemble a virgin-like state. Each developmental stage, as well as the maintenance of each stage, depends on a critical balance between proliferation, differentiation, and cell death orchestrated by multiple signaling pathways. It is widely held that defects, somewhere in this molecular circuitry, can result in the transformation of a mammary cell from a normal state to a tumorigenic state. This tumorigenic state is typified by dysregulated proliferation, differentiation, and programmed cell death of the affected cells. It is also widely held that methods that inhibit proliferation, induce differentiation, and induce cell death of tumor cells are beneficial to the treatment of a tumor. Furthermore, it is widely held that methods that inhibit proliferation, induce differentiation, and potentate cell death can reduce the tumorigenic potential of a population of cells.

2.30 C/EBPβ and the C/EBP Family of Transcription Factors

The study of the molecular circuitry of mammary gland development and its relationship to mammary tumor formation has become a model system for understanding tumorigenesis in general. Important to this molecular circuitry is the expression and biological activities of the transcription factor C/EBPβ. C/EBPβ is one of six currently known members of the CCAAT/enhancer-binding protein (C/EBP) family of transcription factors which includes C/EBPα, C/EBPβ, C/EBPγ, C/EBPδ, C/EBPε, and C/EBPζ. The C/EBP family has a vast array of biological activities including roles in the regulation of development, metabolism, proliferation, differentiation, and inflammation (for review, see Poli (1998) J Biol Chem 273(45):29279-29282, incorporated herein by reference). C/EBP transcription factors are modular proteins comprised of a basic domain-leucine zipper (bZIP) and a transactivation domain. The bZIP enables two compatible bZIP molecules, including various C/EBP family members, to noncovalently bind together through their leucine zipper domains into a dimeric structure. The dimeric form is then capable of noncovalently binding to specific deoxyribonucleic acid (DNA) sequences through interactions with the basic DNA binding domain of the bZIP. Upon binding to a DNA site, the transactivation domain is positioned to interact with other transcriptional machinery to stimulate, or otherwise regulate, gene expression. Additional mechanisms of action including direct protein-protein interactions are also contemplated.

2.40 The Role of C/EBPβ in the Regulation of Mammary Cells

Gene deletion studies show that the transcription factor, C/EBPβ is critical for growth and differentiation of the mammary gland (Gigliotti (1998) J Cell Physiol 174(2):232-239, incorporated herein by reference). C/EBPβ null mice exhibit severely curtailed alveolar development and differentiation during pregnancy. Epithelial cell proliferation in early pregnancy and differentiation at late pregnancy stages are strongly impaired in the absence of C/EBPβ. No expression of β-casein or whey acidic protein (WAP) mRNA is detected in mammary tissue from C/EBPβ null mice late in pregnancy and the mice failed to lactate, showing the influence of C/EBPβ on differentiation. The regenerative response following partial hepatectomy in C/EBPβ null mice shows that DNA synthesis is decreased to 25% of normal and liver regeneration is reduced compared to normal mice demonstrating that C/EBPβ is required for the proliferative response to partial hepatectomy. In addition, increased C/EBPβ expression is associated with apoptosis accompanying involution following lactation.

These data demonstrate multiple roles for C/EBPβ in proliferation, differentiation, and cell death. The literature shows that C/EBPβ plays these roles in numerous tissues including, but not limited to, epithelium, endothelium, lymphoid, and myeloid cells. The literature demonstrates that C/EBPβ regulates proliferation, differentiation, and cell death in various cell types including, but not limited to: mammary epithelial cells, prostate cells, adipose, endothelial cells (including vascular endothelium), epithelial cells, hepatocytes, lymphoid cells (including B-cells and T-cells), and myeloid cells (including monocytes and macrophages).

There has been a lack of understanding regarding how one factor, C/EBPβ, regulates proliferation, differentiation and cell death in the majority of tissues and cells of the body; especially given that these processes are by dogma opposed to one another. Without being bound to mechanism or theory, the inventor has discovered how C/EBPβ regulates proliferation, differentiation, and cell death; how such regulation relates to tumorigenesis and the treatment of tumors and tumorigenesis. The present invention provides compositions and methods for use thereof for the treatment of tumorigenesis, the inhibition of tumorigenesis, the inhibition of cellular proliferation, the induction of cellular differentiation, and the potentiation of cell death.

2.50 Isoforms of C/EBPβ

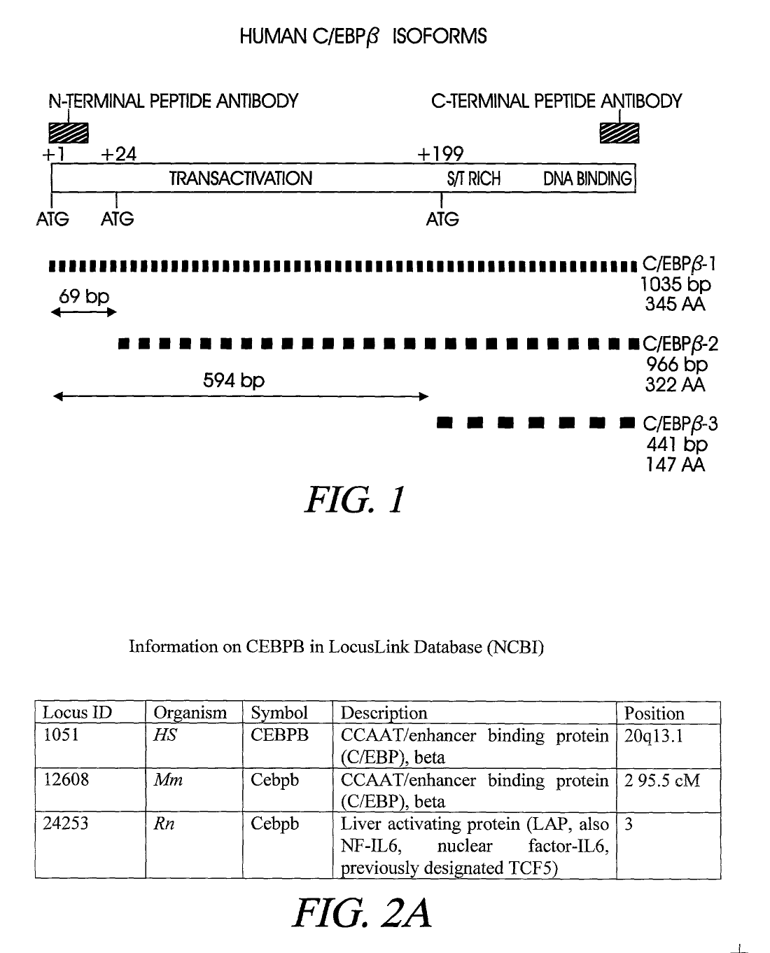

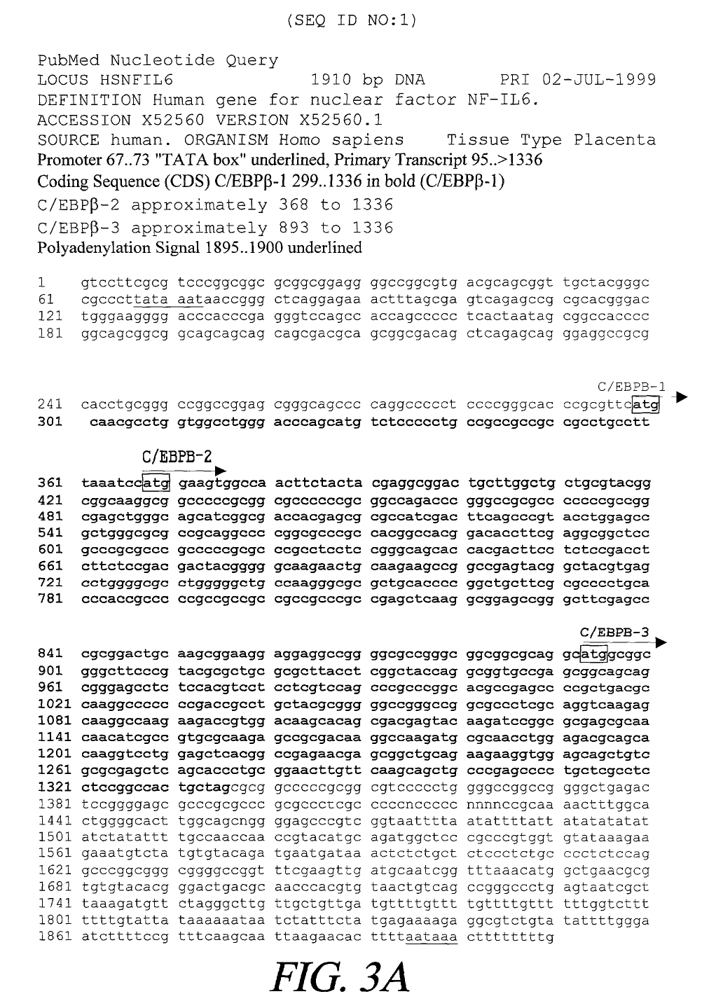

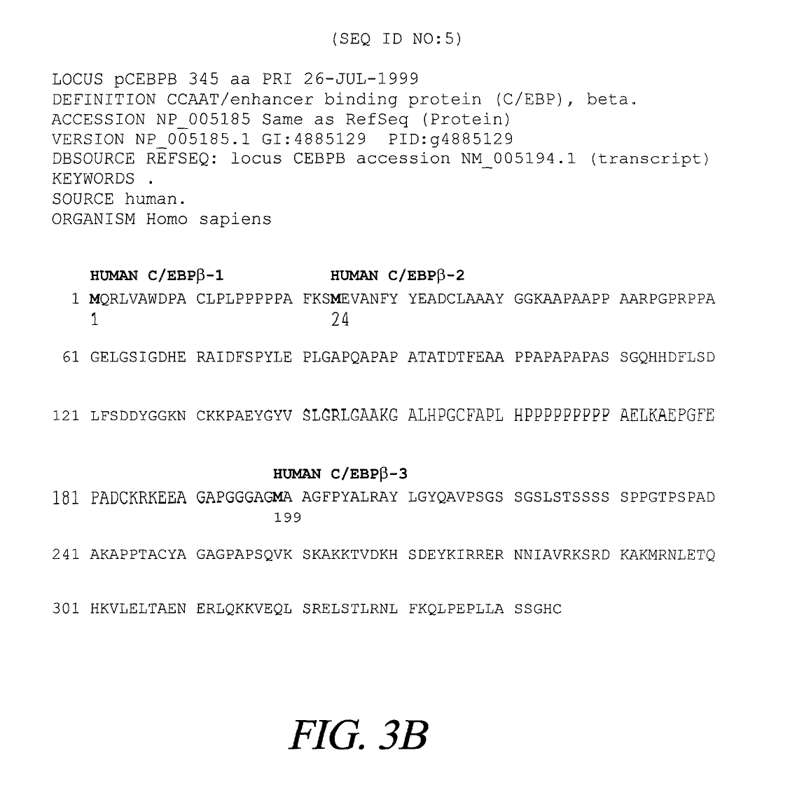

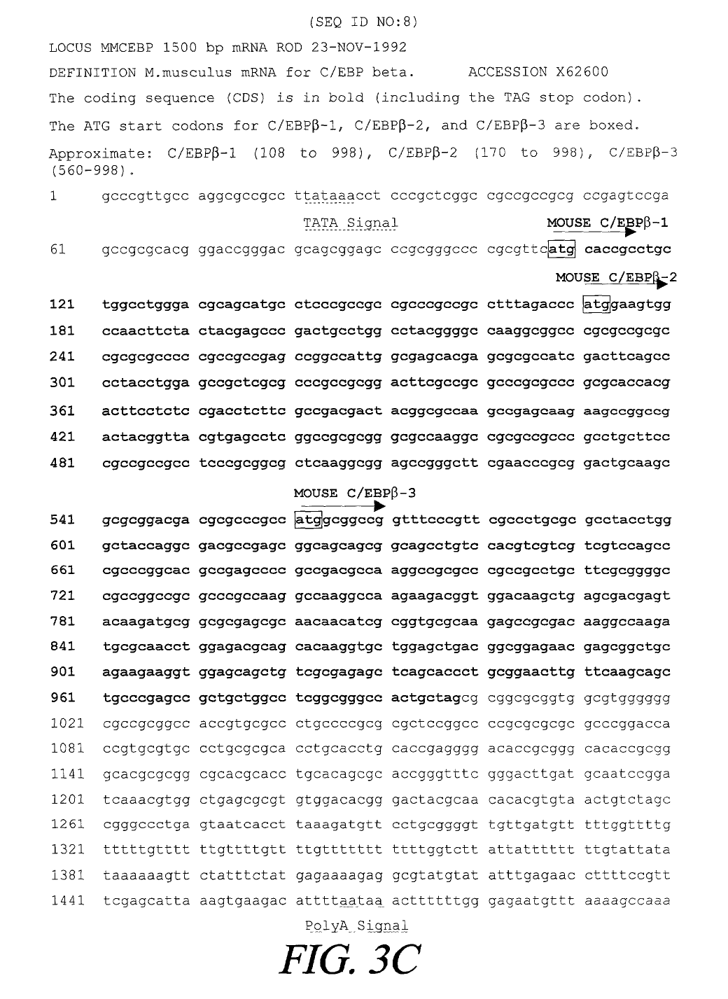

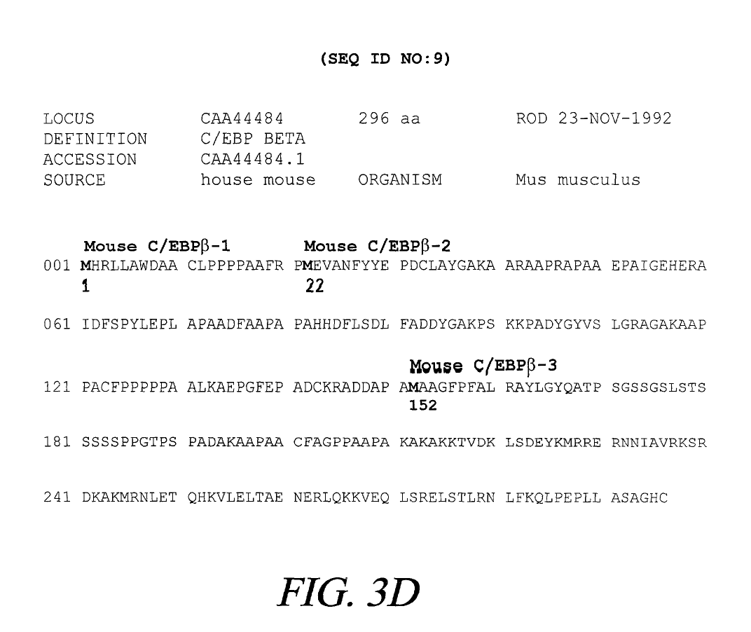

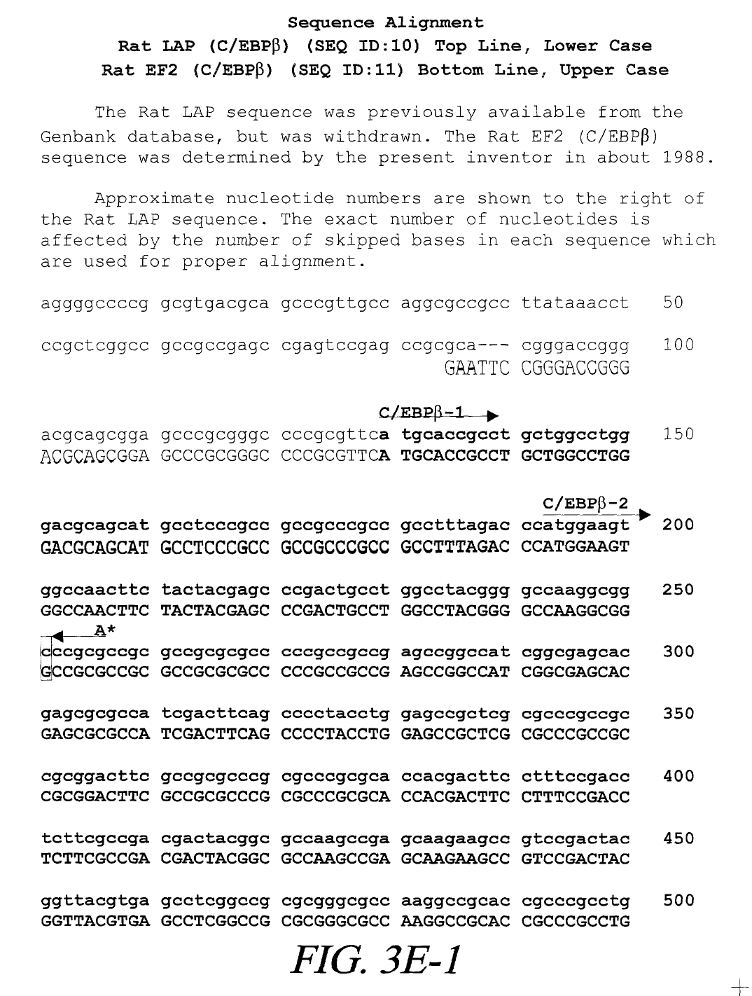

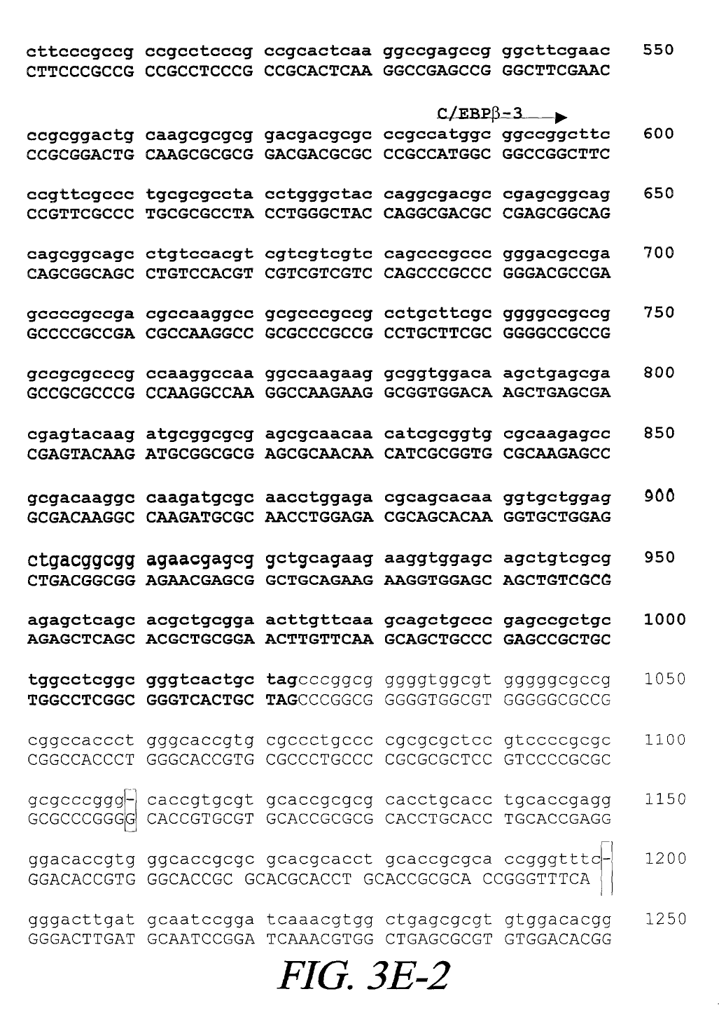

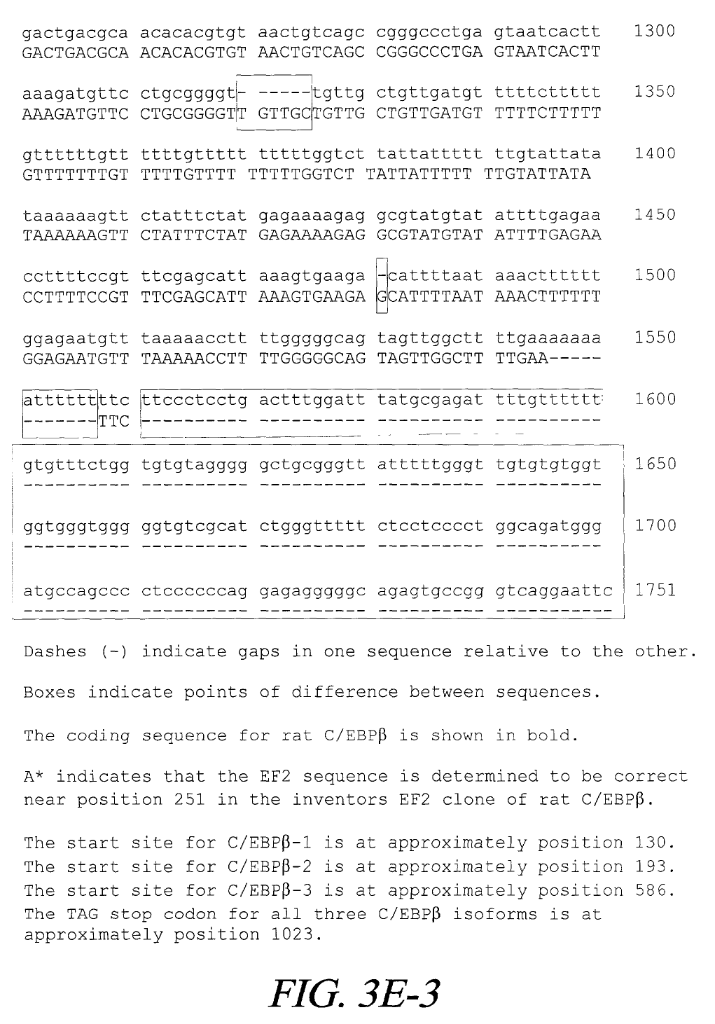

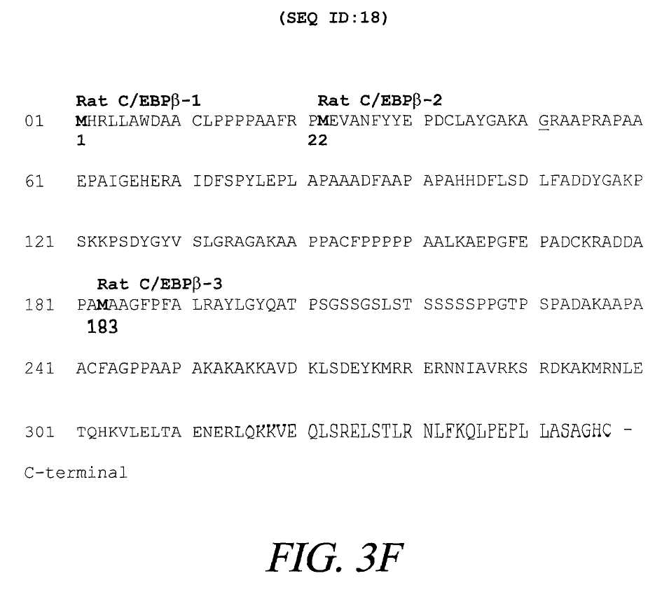

The human, mouse, and rat C/EBPβ genes contain three open reading frames (ORFs) with in-frame ATG translation start sites which correspond to three C/EBPβ isoforms; referred to herein as C/EBPβ-1, C/EBPβ-2, and C/EBPβ-3 (see FIGS. 1, 3A, 3B, 3C, 3D, 3E, and 3F). The first ATG gives rise to C/EBPβ-1 (52 kDa in humans and 42 kDa in mice and rats). The second ATG gives rise to C/EBPβ-2 (45 kDa in humans and 36 kDa in mice and rats). The C/EBPβ-1 isoform is about 23 amino acids longer than the C/EBPβ-2 isoform in human cells and about 21 amino acids longer in mice and rats. The molecular weights are approximate as determined by SDS-PAGE and vary by phosphorylation and other protein modification. The third ATG gives rise to C/EBPβ-3 (20 kDa in both humans and mice). Alternate names used for C/EBPβ-3 include p20 (in reference to a protein with approximate molecular weight of 20 kDa) and liver-enriched transcriptional inhibitor protein (LIP).

Commonly, the C/EBPβ-1 and C/EBPβ-2 isoforms are referred to collectively, in the prior art, as C/EBPβ or as liver-enriched transcriptional activator protein (LAP). That is, no distinction was made between the C/EBPβ-1 and C/EBPβ-2 isoforms in many prior art references. In other instances, the prior art describes the second AUG as the translation initiation start site for the full length protein, LAP, and the first AUG as being in the 5′-untranslated region. In other words, it was either considered that C/EBPβ-1 and C/EBPβ-2 were essentially the same protein with identical functions or that C/EBPβ-1 was not synthesized and that C/EBPβ-2 (LAP) was the full length protein.

As specifically defined and used herein, the present invention distinguishes between C/EBPβ-1, C/EBPβ-2, and C/EBPβ-3 and does not use the terms C/EBPβ-1, C/EBPβ-2, and C/EBPβ-3 interchangeably. The present inventor has determined that C/EBPβ-1 is a differentiation factor, that C/EBPβ-2 is a proliferation factor, and that C/EBPβ-3 inhibits inappropriate cellular proliferation and/or promotes cell death. The present inventor has determined the relationship between the expressions and activities of C/EBPβ-1, C/EBPβ-2, and C/EBPβ-3 that are related to tumorigenesis. Furthermore, the present invention provides methods for using C/EBPβ-1 and C/EBPβ-3 to inhibit tumorigenesis and methods for using C/EBPβ-1 and C/EBPβ-3 as anti-tumor factors.

2.60 Development of an Antibody Specific to the C/EBPβ-1 Isoform

There is a lack of knowledge in the prior art concerning the relative expression and biological activities between C/EBPβ-1 and C/EBPβ-2. A contributing factor to this lack of understanding was the fact that C/EBPβ-1 could not be resolved effectively from phosphorylated C/EBPβ-2 using Western blotting detection methods and the available antibody to the C-terminus of C/EBPβ which binds to all three isoforms (see FIG. 1).

The inventor developed a rabbit polyclonal antibody to the first 16 amino acids in the N-terminus (SEQ ID NO:14) of the human C/EBPβ-1 isoform polypeptide (SEQ ID NO:5). These amino acids are not present in the human C/EBPβ-2 isoform polypeptide (SEQ ID NO:6) (see FIG. 1). This polyclonal antibody was developed using techniques known to one of ordinary skill in the art (injection into rabbits with adjuvant and affinity purification). The 16 amino acid sequence was generated by solid phase peptide synthesis as is known in the art. With this antibody, it was possible to design experiments to differentiate the expression patterns and biological activities of the C/EBPβ isoforms, especially between C/EBPβ-1 and C/EBPβ-2. (The C-terminal antibody is commercially available from Santa Cruz Biotechnology, Inc., Santa Cruz Calif. 95060, USA. However, the inventor has also developed a C-terminal C/EBPβ antibody which is used in certain experiments.) A rat or mouse C/EBPβ-1 specific N-terminal antibody could be developed using the same standard techniques.

3.00 Distinct Expressions and Activities of the C/EBPβ Isoforms

C/EBPβ-1 is disclosed in the present invention to be an anti-tumor agent, to promote cellular differentiation, and to inhibit cellular proliferation, It is discovered that, in general, C/EBPβ-1 is abundantly expressed in differentiated cell types. It is further disclosed in the present invention that the expression and/or activity of C/EBPβ-1 is typically reduced or lost during tumorigenesis.

In certain embodiments, C/EBPβ-2 is disclosed in the present invention to be a tumor promoter, especially when C/EBPβ-2 is expressed in the nucleus. C/EBPβ-2 is disclosed in the present invention to generally promote cellular proliferation, to be abundantly expressed in proliferating cell types (with essentially cytoplasmic expression in normal cells in a normal environment). A nuclear expression pattern of C/EBPβ-2 is disclosed to be consistently, but not necessarily, gained during tumorigenesis. In certain embodiments, increasing levels of phosphorylation of C/EBPβ-2 is disclosed to result in increasing tumorigenicity.

C/EBPβ-3 was known in the art to inhibit transactivation of a reporter gene by C/EBPβ-2 in cell culture (Descombes et al. (1991) Cell 67:569-579, incorporated herein by reference). However, C/EBPβ-3 was widely believed to be a tumorigenic factor (see, e.g., Rosen et al., (1998) Biochem. Soc. Symp. (63)101-13 and Zahnow et al., (1997) J Natl. Cancer Inst. 89(24):1887-91. It is disclosed in the present invention that C/EBPβ-3 is an anti-tumor agent. In certain embodiments, the expression and/or a biological activity of C/EBPβ-3 is lost during tumorigenesis.

Except as stated otherwise, these discoveries were made using molecular cloning, expression, and protein analysis techniques known to one with skill in the art. Development of the C/EBPβ-1 specific antibody enabled the inventor to experimentally identify differences between the expression patterns and biological activities of the C/EBPβ-1 and C/EBPβ-2 isoforms (which differ by only 23 amino acids in human cells) which were indistinguishable previously especially because phosphorylated C/EBPβ-2 peptide co-migrates with C/EBPβ-1 peptide on SDS-PAGE/Western blots. In certain analyses, standard membrane stripping protocols are used to remove signal from the C-terminal antibody and the membranes are reprobed with the C/EBPβ-1 specific N-terminal antibody.

3.10 C/EBPβ-1 Promotes Differentiation, Inhibits Proliferation, and is an Anti-Tumorigenic Factor

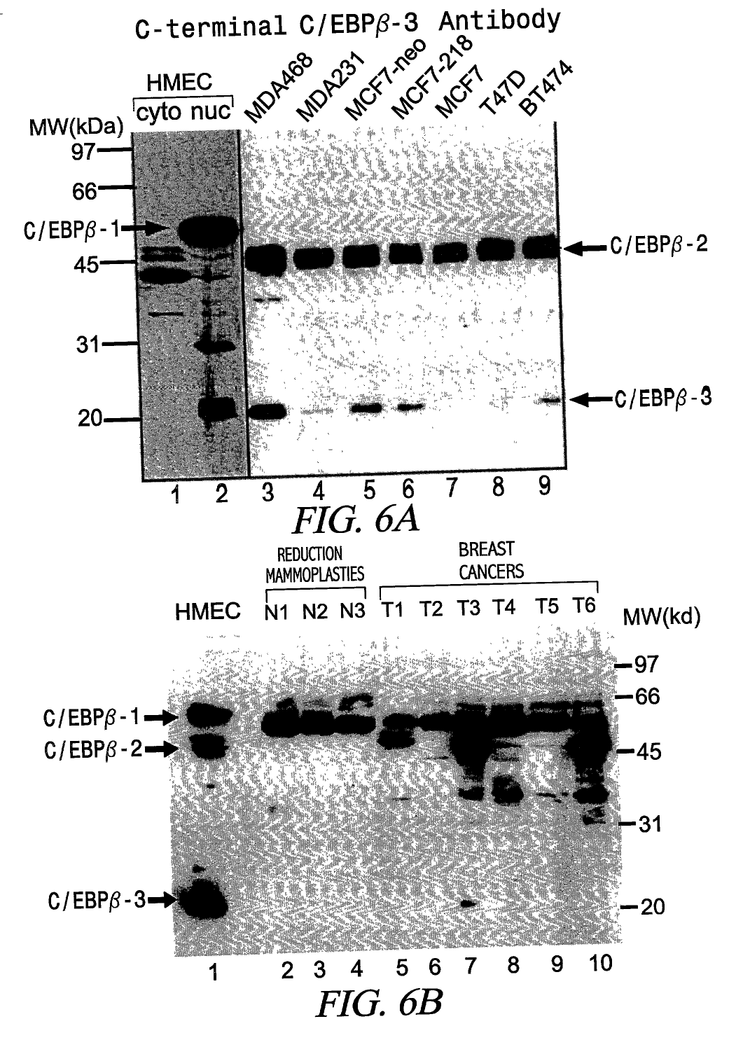

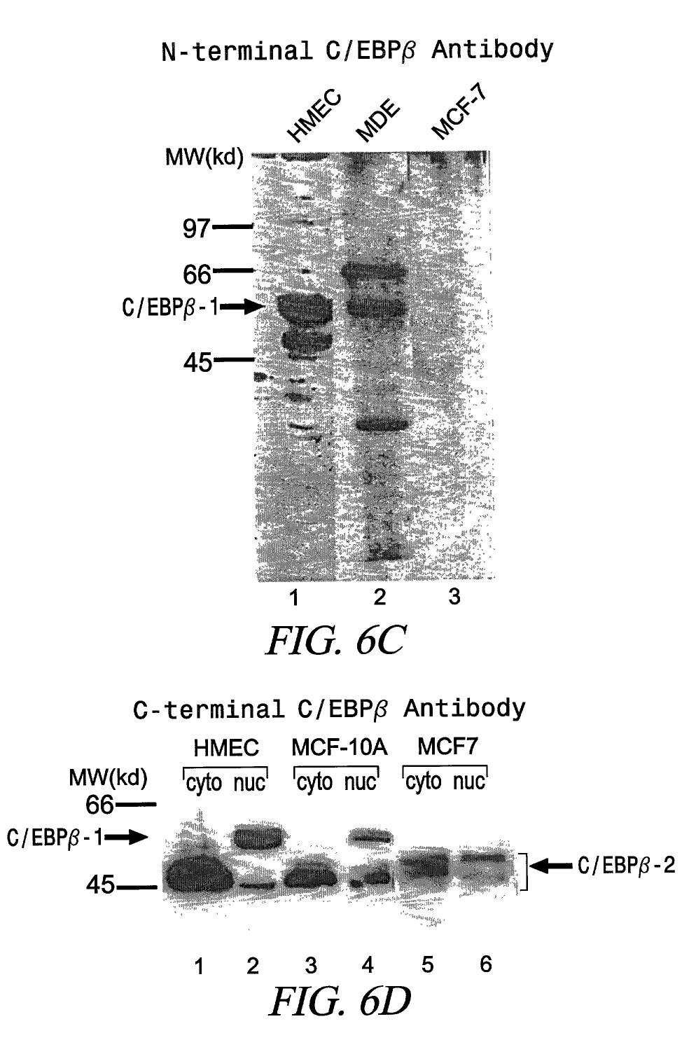

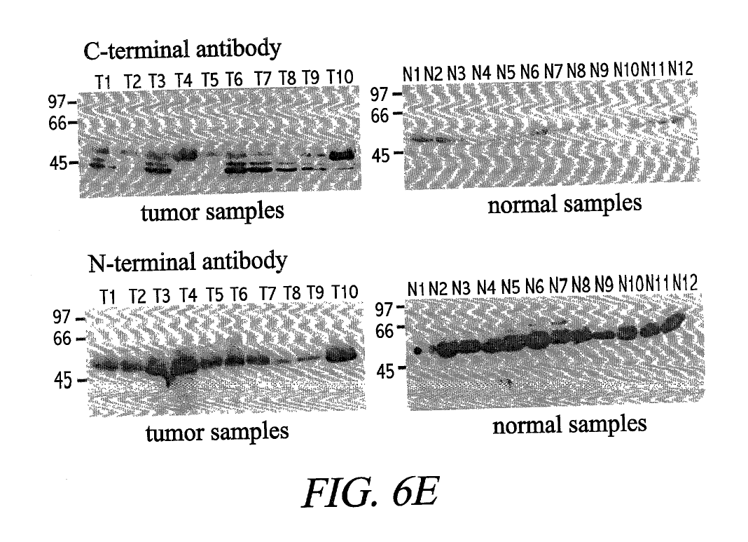

It is a discovery of the present invention, that C/EBPβ-1, but not C/EBPβ-2, promotes differentiation and inhibits proliferation. The Western blot in FIG. 6A shows the relative expression patterns of C/EBPβ-1 in human mammary epithelial cells (HMECs) compared to a panel of seven mammary carcinoma cell lines: MDA468, MDA 231, MCF7-neo, MCF7-218, MCF7, T47D, and BT474. The MDA468, MDA 231, MCF7, T47D, and BT474 cell lines, are available from the American Type Culture Collection (ATCC, Rockville, Md.).

HMECs are primary epithelial cells derived from breast reduction surgery and placed into cell culture with a cocktail of growth factors (epidermal growth factor (EGF), insulin, hydrocortisone, and pituitary extract) to stimulate proliferation of the cells. In the normal, developed, non-pregnant, non-lactating mammary gland; mammary epithelial cells essentially do not proliferate and are differentiated. The growth factors used to culture the HMECs alter the balance of differentiation versus proliferation causing the HMECs to proliferate, although slowly compared to many other cell types. The lifespan of HMECs in culture is about 40 passages; thus, HMECs are not transformed or immortalized. The HMECs provide a model system of partially-differentiated, slowly-proliferating epithelial cells.

The panel of seven mammary carcinoma cell lines provides an extensive model system of rapidly proliferating epithelial cells that are tumorigenic and immortal. (The BT474 cells are slower growing, but still immortal and tumorigenic.) The tumorigenic nature of these cell lines is demonstrated by the formation of tumors upon implantation into nude mice. In addition, the cell lines are clonal. This means they are selected from single cells which are expanded clonally. One importance of the clonal nature of the panel of mammary carcinoma cell lines, is that they provide a model for a tumorigenic cell that is free from contamination with non-tumorigenic cells. This is in contrast to tumor biopsy samples which contain both tumor cells and normal cells (see infra).

Turing back to the expression of C/EBPβ-1 in HMECs versus the panel of mammary carcinoma cells as depicted in FIG. 6A. C/EBPβ-1 expression is observed in the HMECs, but C/EBPβ-1 expression is not observed in any cell line from the panel of mammary carcinomas (confirmed by reprobing with C/EBPβ-1 specific N-terminal antibody). Additionally, cell fractionation shows that the C/EBPβ-1 expression in the HMECs is essentially nuclear with essentially little or no cytoplasmic C/EBPβ-1 expression (FIG. 6A, lanes 1 and 2). Very significantly, these data show that C/EBPβ-1 expression is lost during tumorigenesis (FIG. 6A, lanes 3-9). Thus, the model system for non-tumorigenic, highly differentiated, slowly proliferating epithelial cells (HMECs) displays C/EBPβ-1 expression; while the model system for undifferentiated, proliferating, transformed or tumorigenic epithelial cells (the panel of mammary carcinomas) displays a loss of C/EBPβ-1 expression.

Epithelial cells from reduction mammoplasty are similar to the HMECs except that they are not cultured with exogenous growth factors. In other words these cells are sampled directly from non-proliferating mammary epithelium and serve as an exemplary model system for differentiated, non-proliferating epithelial cells. Western blots of epithelial cells from reduction mammoplasties show that C/EBPβ-1 is highly expressed in this cell type (FIG. 6B, lanes 2, 3, and 4).

Another model for differentiated epithelial cells is the milk ductal epithelial cell (MDE). These cells are sloughed off during lactation and are recently rapidly proliferating cells which have just fully differentiated. FIG. 6C is a Western blot of C/EBPβ-1 expression in HMECs, MDEs, and MCF-7 (breast carcinoma) cells probed with the N-terminal C/EBPβ-1 specific antibody. The data in FIG. 6C show that C/EBPβ-1 is highly expressed in the HMEC and MDE cells, but C/EBPβ-1 expression is lost in the breast carcinoma cells. In addition a second highly phosphorylated form of C/EBPβ-1 is identified by the N-terminal C/EBPβ-1 specific antibody in the MDE cells with an apparent molecular weight on SDS-PAGE of 64 kDa (FIG. 6C, lane 2). The phosphorylation state of the 64 kDa band is being confirmed by treatment with phosphatase by methods known to one with skill in the art.

Contrary to the observation that C/EBPβ-1 expression is lost in the panel of seven mammary carcinoma cell lines, it is noted that the samples from tumors in FIG. 6B (lanes 5 to 10) and FIG. 6D (both left panels) appear to express C/EBPβ-1. However, it is believed that the tumor cells have actually lost C/EBPβ-1 expression; a result which is masked in the data by contamination of the primary tumor cells with normal cells. This contamination with normal cells is inevitable when assaying a surgically removed specimen.

Several methods can be used to determine that C/EBPβ-1 expression is lost in the tumor specimens of FIGS. 6B and 6D. The primary cultures are grown in culture for several generations after which the normal mammary cells in the cultures die while the tumor cells, being transformed, survive. In another method, in situ hybridization of mixed cell populations confirms that C/EBPβ-1 expression is localized to normal cells, but absent in tumor cells. The in situ hybridization is performed according to methods know to one with skill in the art using a C/EBPβ1 specific antibody. C/EBPβ-1 is not observed in epithelial derived tumor cells which are isolated away from normal cells (FIG. 6A, lane 3-9).

The cell line MCF-10A is a model of an intermediate stage in the progression through tumorigenesis. MCF-10A was developed by growing normal mammary epithelial cells in culture under conditions of ultra-low calcium. A spontaneous transformation took place and was clonally expanded as the MCF-10A cell line. MCF-10A has the intermediate properties of being immortal in cell culture, but incapable of forming tumors in nude mice. A comparison of cytoplasmic and nuclear C/EBPβ-1 expression between HMECs (non-tumorigenic), MCF-10A cells (engaged in the tumorigenic process), and MCF7 cells (aggressively tumorigenic cells) is made in FIG. 6D. FIG. 6D shows that HMECs express a high level of C/EBPβ-1 in the nucleus, MDA 231s express essentially zero C/EBPβ-1, and MCF-10 As express an intermediate amount of nuclear C/EBPβ-1. Thus, a progressive loss of C/EBPβ-1 expression occurs during tumorigenesis (FIG. 6D).

The data of Gigliotti (1998), supra, demonstrate that C/EBPβ (LAP) expression/activity is fundamental to both the proliferation and the differentiation of epithelial cells (including mammary and liver epithelial cells). However, it was not understood how C/EBPβ directed both proliferation and differentiation, two processes that are opposed to one another by dogma. The present inventor shows that the C/EBPβ-1 isoform of C/EBPβ is the differentiation factor and is lost during tumorigenesis. Thus, C/EBPβ-1 an anti-tumor factor. It is an aspect of the present invention that the introduction of C/EBPβ-1 to a tumor cell is a treatment for a tumor.

3.20 C/EBPβ-2 Promotes Proliferation: Relationship to Tumorigenesis

It is a discovery of the present invention, that C/EBPβ-2, but not C/EBPβ-1, promotes proliferation. The Western blot in FIG. 6A shows the relative expression patterns of C/EBPβ-2 in HMECs and the panel of mammary carcinoma cell lines. Very little, if any, C/EBPβ-2 is expressed in the nuclei of HMECs (FIG. 6A, lane 2) and a moderate amount is observed in the cytoplasmic preparations of the HMECs (FIG. 6A, lane 1). In contrast, high level expression of C/EBPβ-2 is observed in each of the panel of mammary carcinoma cell lines (FIG. 6A, lanes 3-9). Thus, the model system for non-tumorigenic, highly differentiated, slowly proliferating epithelial cells (HMECs) displays relatively moderate C/EBPβ-2 expression which is almost exclusively compartmentalized in the cytoplasm; while the model system for undifferentiated, proliferating, transformed epithelial cells (the panel of mammary carcinomas) displays a gain of high level C/EBPβ-2 expression. It is contemplated that cytoplasmic expression of C/EBPβ-2 does not stimulate proliferation, at least strongly, because the compartment of C/EBPβ-2 action is contemplated to be the nucleus (without being bound to mechanism).

Examination of samples from reduction mammoplasty reveal that C/EBPβ-2 is not expressed in any compartment of these cells (FIG. 6B, lanes 2-4). Thus, the present inventor has determined that there is a lack of C/EBPβ-2 expression in this exemplary model of differentiated, non-proliferating epithelial cells. It is quite significant that cells from reduction mammoplasty lack C/EBPβ-2 expression.

In contrast to the differentiated HMECs and cells from reduction mammoplasty, there is a gain of C/EBPβ-2 expression in nearly every breast tumor examined and the level of C/EBPβ-2 expression correlates with the pathological severity of the tumors (FIG. 6B lanes, 5-10; FIG. 6D, tumor samples, C-terminal antibody).

Data for the model of progressive tumorigenicity is shown in FIG. 6D. There is a progressive gain of nuclear C/EBPβ-2 expression from HMECs (mostly differentiated, lane 2) to MCF-10As (immortalized, but not tumorigenic in nude mice, lane 4) to MCF7s (immortalized and aggressively tumorigenic, lane 6). Furthermore, the nature of the C/EBPβ-2 in the nucleus changes from not phosphorylated in the HMECs (lane 2) to partially phosphorylated in the MCF-10As (lane 4, the middle and the upper of the three bands in the C/EBPβ-2 bracket) to highly phosphorylated in the MCF7s (lane 6, upper band in C/EBPβ-2 bracket). Thus, the present inventor has discovered that the gain of nuclear C/EBPβ-2 expression is a key step in tumorigenesis. Further, the inventor has identified that the phosphorylation state of nuclear C/EBPβ-2 might be a component of tumorigenesis. Note, without being bound to mechanism, it is also contemplated that the C/EBPβ-2 expression seen in the nucleus of HMECs results from contamination between the nuclear and cytoplasmic fractions. It is contemplated that the cytoplasmic fraction of C/EBPβ-2 is not phosphorylated while the gain of phosphorylation is a cellular signal that moves the C/EBPβ-2 into the nucleus (FIG. 6D, lanes 2, 4, and 6).

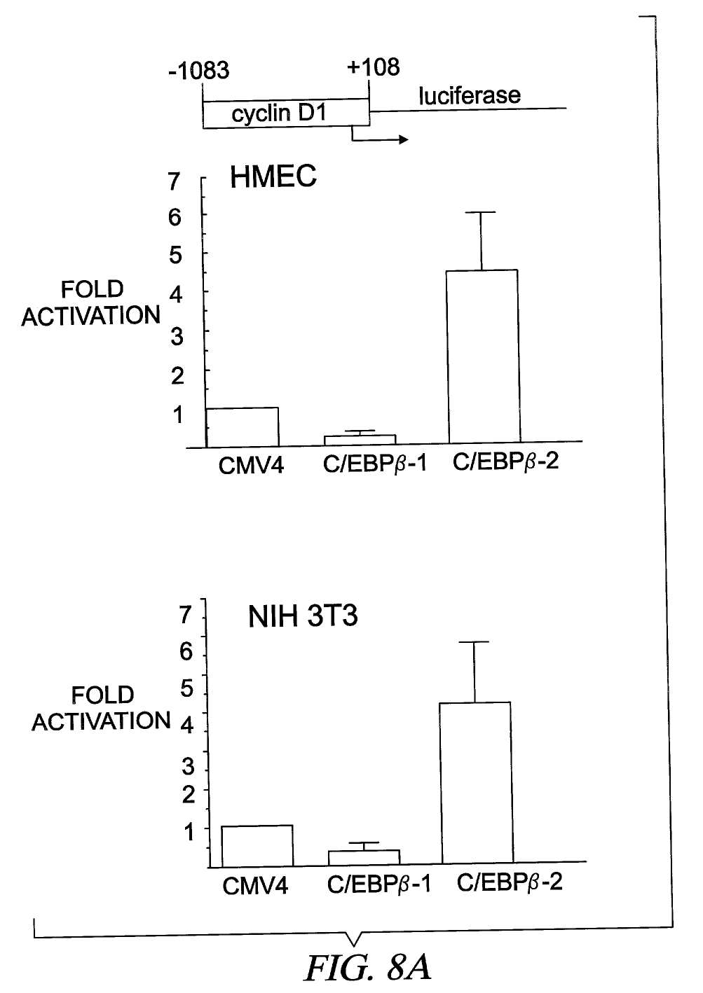

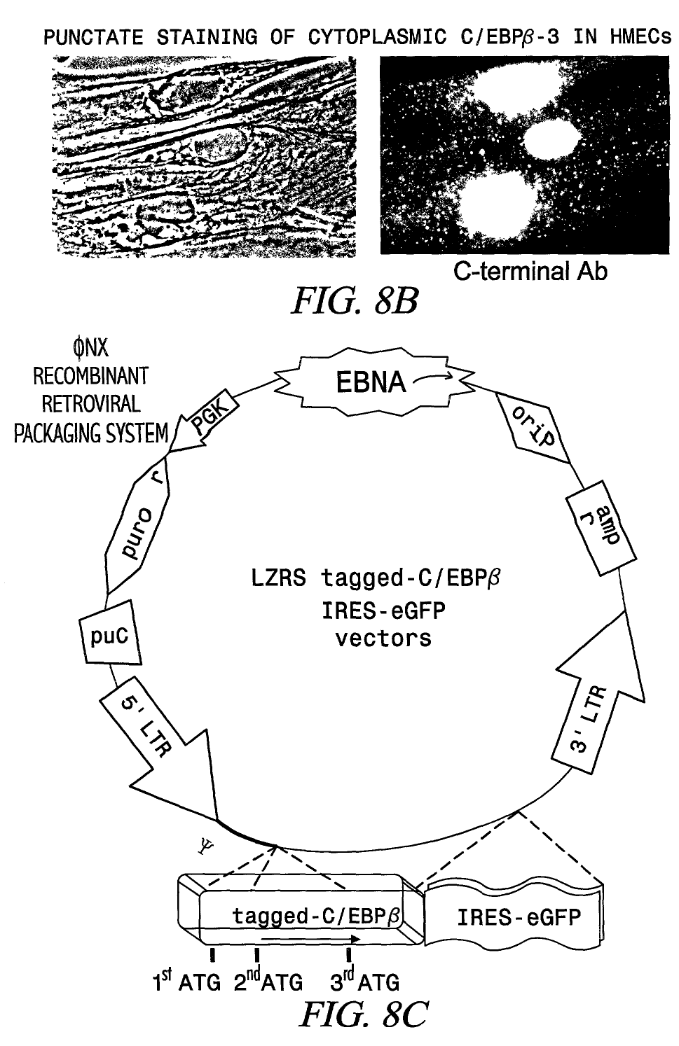

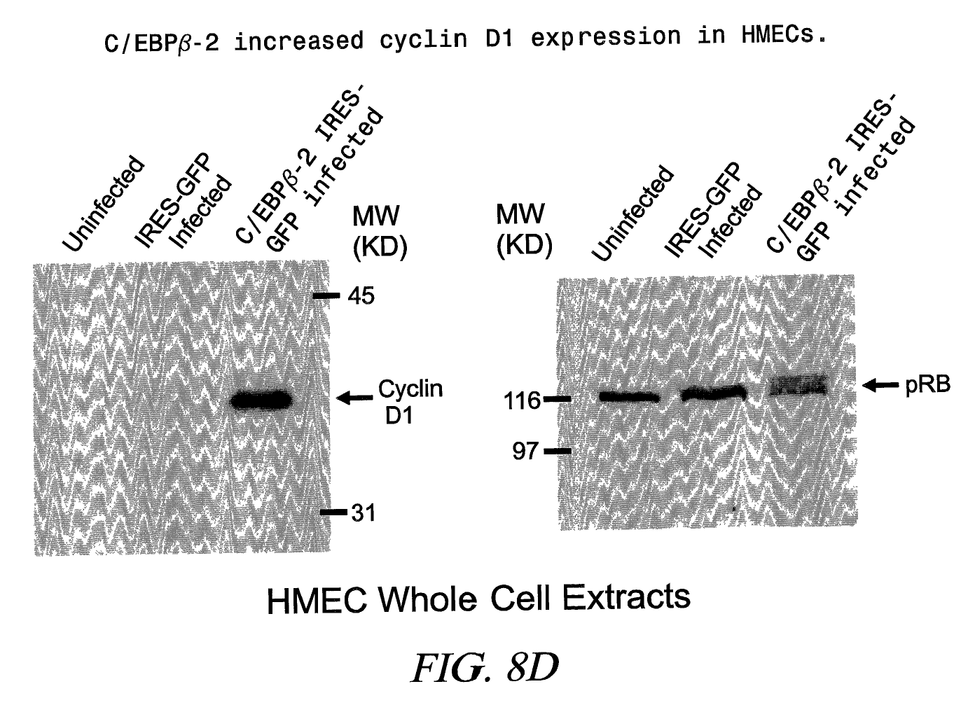

The cytoplasmic localization of C/EBPβ-2 is confirmed in FIG. 8B. The top micrograph shows a phase microscopy view of a field of human mammary epithelial cells (HMECs) growing in cell culture. The bottom micrograph shows an immunofluorescence microscopy view of the same field of HMECs stained with a C-terminal C/EBPβ antibody (which recognizes all three isoforms of C/EBPβ) and detected with Alexa 546 (which fluoresces red) conjugated secondary antibody. Punctate staining of C/EBPβ-2 was observed in the cytoplasm. Intense staining of C/EBPβ-1 and C/EBPβ-3 in the nucleus was also observed as expected.

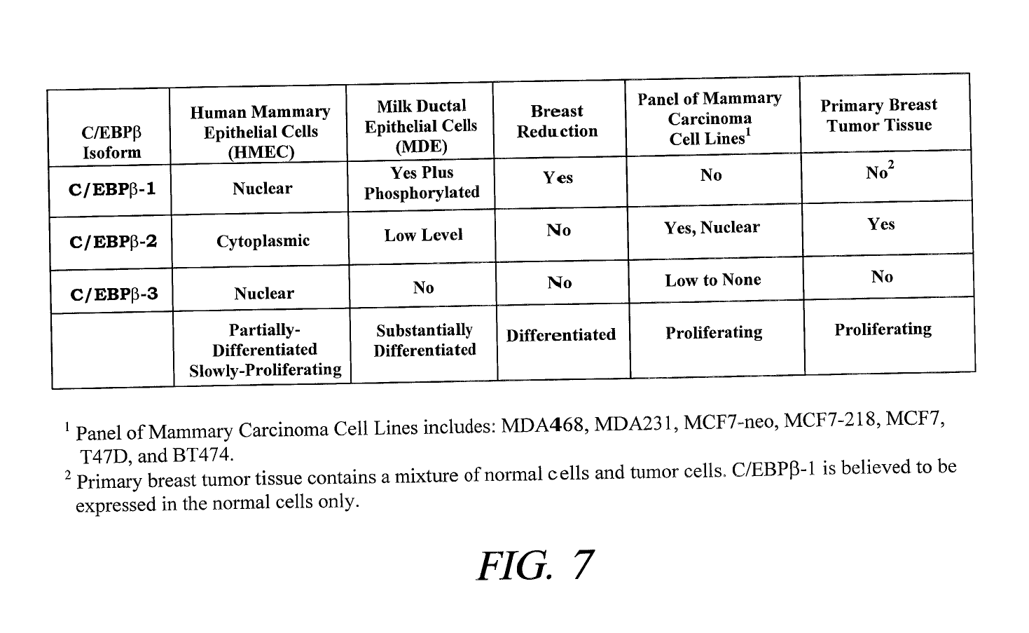

The observation that C/EBPβ-2 is expressed in HMECs, at least in the cytoplasmic compartment (FIGS. 6A and 6B), suggests that C/EBPβ-2 expression is a mechanism of normal cellular proliferation induced by the addition of growth factors (see above). Also, a low level of C/EBPβ-2 expression is observed in MDE cells (data not shown) which is contemplated to be residual expression from recent normal cellular division. Further, C/EBPβ-2 expression is characteristic of all proliferating cell types (data not shown) including, but not limited to: fibroblasts (NIH3T3), cervical (HeLa), epidermal (A431), kidney (Cos), B-cell (BK3a), hepatocytes (H4IIE), prostate (pC3 and DV145). However, C/EBPβ-2 is sequestered in the cytoplasm of non-tumor cells that are stimulated to proliferate in a normal environment (HMECs, FIG. 6A and 7). Thus, C/EBPβ-2 expression, especially when the majority of the expression is cytoplasmic, is contemplated to be a marker of normal cellular proliferation; but the gain of nuclear, and particularly phosphorylated C/EBPβ-2, is contemplated to be tumorigenic (without being bound to mechanism). Under normal circumstances of proliferation, it is contemplated that the phosphorylation state of the C/EBPβ-2 is held in check (de-phosphorylated) and that such regulation breaks down during tumorigenesis including the loss of C/EBPβ-1 expression (or activity), supra, and/or the loss of C/EBPβ-3 expression (or activity), infra.

3.21 C/EBPβ-2, but not C/EBPβ-1, Activates the Cyclin D1 Promoter

Without being bound to this mechanism, it is contemplated that one mechanism through which C/EBPβ-2 acts as a proliferation factor is through the upregulation of cyclin D1 expression. Cyclin D1, in turn, is a critical cell cycle factor and a potent stimulator of cellular proliferation. As mentioned previously, the overexpression of cyclin D1 is observed in about 50% of all breast cancers.

In terms of the biological activities, C/EBPβ-2, but not C/EBPβ-1, stimulates the expression of cyclin D1 responsive promoter elements (FIG. 8A). The experiment for FIG. 8 was performed as follows. A luciferase reporter gene construct was made with approximately 1000 basepairs of human cyclin D1 promoter operably linked to the luciferase reporter gene. Additional plasmid vectors were made using the CMV-4 vector with either a C/EBPβ-1 or C/EBPβ-2 specific insert. The CMV-driven C/EBPβ-1 expression vector was constructed by mutating the second in frame methionine to valine and creating a perfect Kozak sequence around the first ATG by standard site-directed mutagenesis procedures to ensure selective expression of C/EBPβ-1 from the plasmid.

Thus, in this experiment, CMV driven C/EBPβ-1 or C/EBPβ-2 expression was constitutive after transfection into HMECs, but luciferase expression was dependent upon stimulation of a cyclin D1 promoter or enhancer binding factor. It was discovered that C/EBPβ-2, and not C/EBPβ-1, was capable of stimulating cyclin D1 promoter activity approximately five fold. In contrast, C/EBPβ-1 expression resulted in more than a two fold decrease in cyclin D1 promoter activity. The level of C/EBPβ-1 and C/EBPβ-2 expression in these experiments was comparable as determined by immunoblotting with an antibody to the epitope tag (6× histidine) which both C/EBPβ isoform constructs express. Moreover, the C/EBPβ-1 protein was active because this expression construct was capable of activating the RSV LTR in transient cotransfection experiments (data not shown).

Since C/EBPβ-1 failed to activate the cyclin D1 promoter the ability of C/EBPβ-1 to bind the promoter was examined. In data not shown, it was determined that C/EBPβ is likely to bind the cyclin D1 promoter at a specific recognition sequence at −549 in the promoter DNA. To examine the C/EBPβ-1 binding, a nuclear extract from HMECs was fractionated by SDS gel electrophoresis and protein in the gel were transferred to an immobilon filter. The filter was cut into slices, and protein eluted from each slice were tested for DNA binding activity. A parallel lane of protein was subjected to immunoblotting with either C-terminal or N-terminal antibodies (against C/EBPβ and C/EBPβ-1,respectively) to detect the location of the C/EBPβ-1 and C/EBPβ-2 isoforms. Protein binding to the −549 cyclin D1 site was recovered in the 55-58 kD fraction and 42-24 kD fraction, respectively (data not shown). Moreover, both the binding activities in the 55-58 kD fraction and 42-24 kD fraction were specifically supershifted by C/EBPβ antibody (data not shown). Thus, it appears that both C/EBPβ-1 and C/EBPβ-2 are capable of binding to the −549 site, and yet only C/EBPβ-2 results in transactivation. Although, the reason for their differential function is not yet known, multiple transcription factors (such as Ap1, E2F, E box/myc and TCF) bind to and regulate the cyclin D1 promoter. It is likely that C/EBPβ-2 can participate in certain protein-protein interactions required for transactivation at the cyclin D1 promoter that C/EBPβ-1 cannot.

3.22 C/EBPβ-2 Overexpression Activates the Endogenous Cyclin D1 Promoter

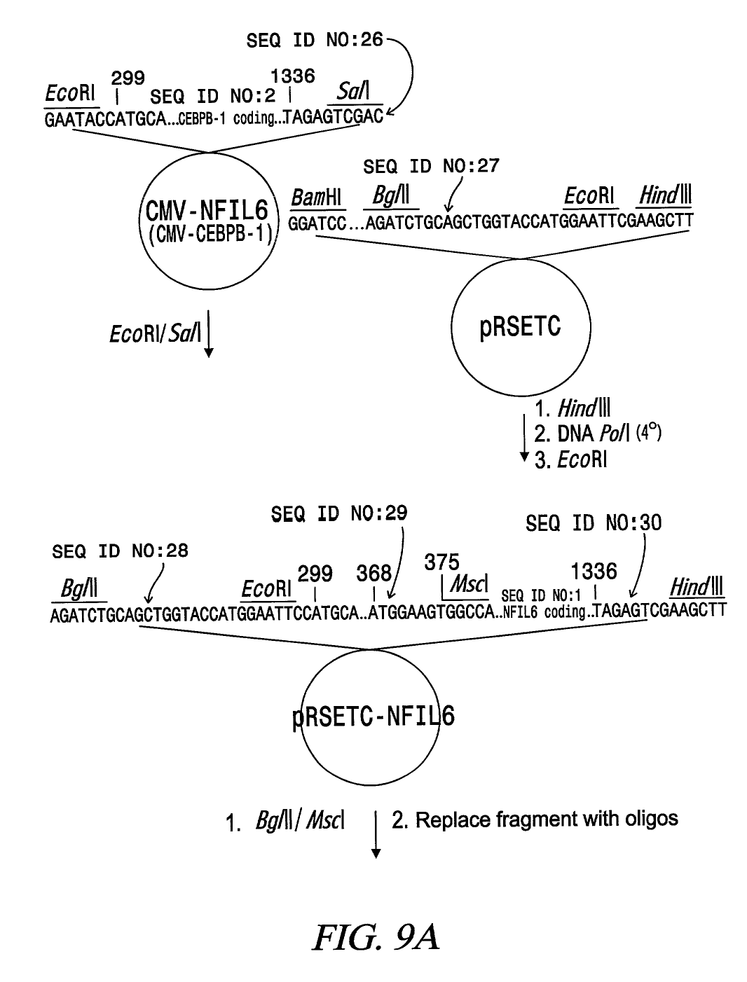

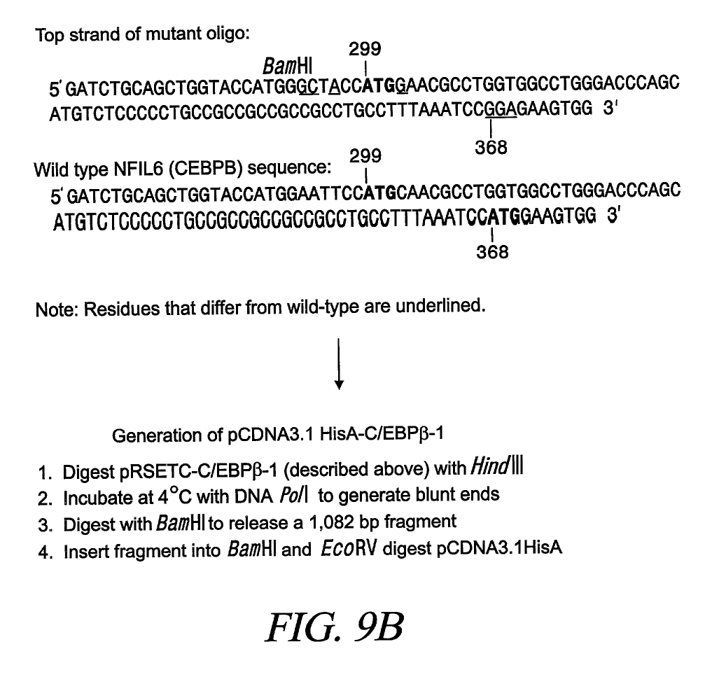

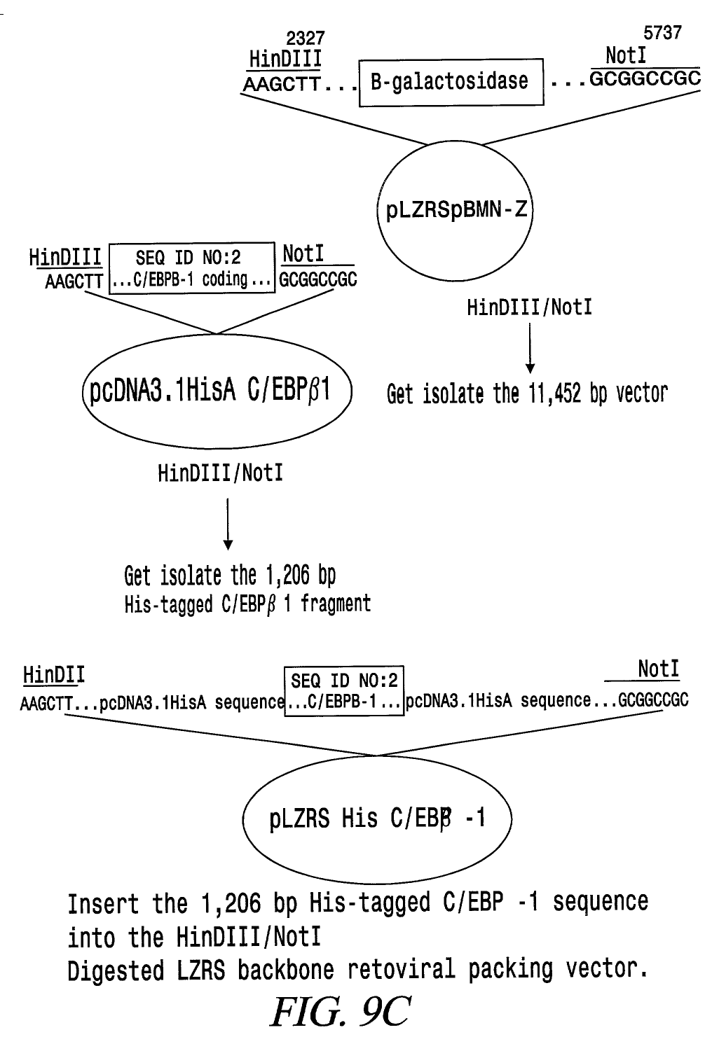

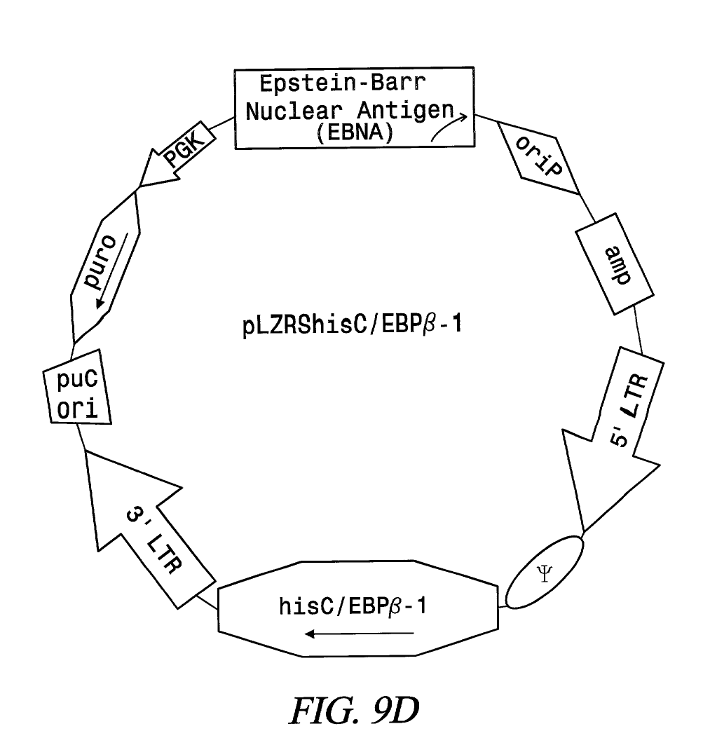

Further experiments were carried out wherein it was determined that C/EBPβ-2 overexpression activates the endogenous cyclin D1 promoter. A vector, pLZRShis-C/EBPβ-2, similar to that shown in FIG. 9D except that the insert was constructed for the expression of C/EBPβ-2 and not C/EBPβ-1,was modified to aid in identifying an quantifying the number of cells infected by administration of the hybrid retroviral vector. An internal ribosome entry sequence (IRES) linked to a green fluorescent protein (GFP) coding region was inserted downstream of the C/EBPβ-2 isoform to arrive at the vector shown in FIG. 8C. Because both C/EBPβ-2 and GFP are expressed from a single viral mRNA the correspondence of coexpressing cells is essentially 100%. Therefore, GFP can be used as a marker for tagged- C/EBPβ-2 expressing cells. It is important to note that C/EBPβ-3 cannot be translated from this C/EBPβ-2 construct because expression of C/EBPβ-3 depends upon a small (nine amino acid) evolutionarily conserved alternative open reading frame (ORF) located before the second in frame ATG of C/EBPβ. Loss of this nine amino acid ORF is sufficient to eliminate C/EBPβ-3 expression.

HMECs were infected with 2×10⁶ infectious units of his-C/EBPβ-2-IRES-GFP virus or virus expressing GFP only (moi=10). It was determined that approximately 70% of the population was infected (GFP positive cells). A Western blot performed on cell extracts obtained 72 hours after viral infection demonstrated that C/EBPβ-2 expression in these normal cells (not immortal and non-tumor forming when transplanted into nude mice) resulted in strong induction of cyclin D1 protein (see, FIG. 8D, left panel). Since one known activity of cyclin D1 is the downstream phosphorylation of the tumor suppresser Rb, the immunoblot was stripped and reprobed with antibody to the retinoblastoma protein (Rb). Increased phosphorylation of pRB was observed in the HMECs with the overexpression of C/EBPβ-2 (see, FIG. 8D, right panel).

3.23 C/EBPβ-2 Overexpression Transforms MCF-10A Mammary Cells

The influence of C/EBPβ-2 overexpression on cell growth was evaluated. Elevated C/EBPβ-2 expression (via the hybrid retroviral vector) in HMECs lead to a more rapid onset of senescence in these cells. However, these data are interpreted in view of well known knowledge that the introduction of oncogenic Ras into primary human fibroblasts provokes premature cell senescence, dependent on both p53 and p16 expression. HMECs are primary, non-immortalized cells, also. Therefore, the senescence of the HMECs upon introduction of C/EBPβ-2 is expected in HMECs.

MCF-10A is a normal, but spontaneously immortalized human mammary epithelial cell line. It is normal, for example, in that it is considered non-tumorigenic in nude mice (an in vivo model for tumorigenesis) and lacks anchorage-independent growth in culture (an in vitro model for tumorigenesis). The karyotype is near diploid and the cells do not contain SV40 genetic information. The parent MCF-10F cells, from which 10A cells were derived, has undergone a t(3:9) translocation resulting in homozygous deletion of the chromosomal subregion 9p21-22 which includes p16. MCF-10F cells are also known to carry an insertional mutation at codon 247 of the p53 gene.

MCF-10A cells were infected with high titer LZRS-his-C/EBPβ-2 retrovirus (2×10⁶ infectious units). Expression of his-C/EBPβ-2 was examined by immunofluorescence microscopy with T7 tag antibody. FITC conjugated T7 tag antibody is sold by Invitrogen 1600 Faraday Ave., Carlsbad, Calif. 92008 and Novagen, 601 Science Dr., Madison, Wis., 53711. The T7 tag is well known in the art and incorporated into the expression vector construct, see, e.g., Invitrogen pRset series of vectors.

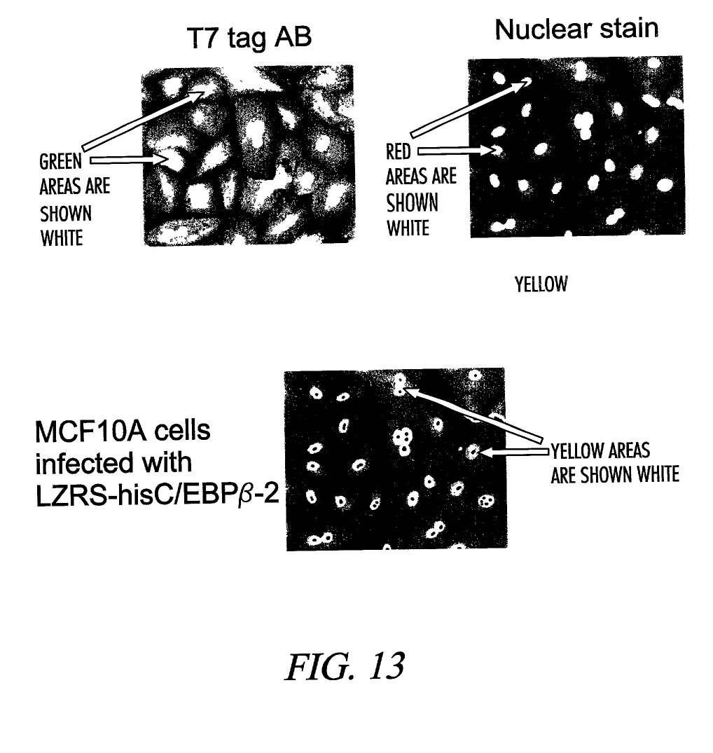

The top left panel in FIG. 13 shows T7 tag staining by immunofluorescence microscopy with FITC conjugated (green) T7 tag antibody (indicating that essentially all cells in the field are infected, also punctate staining of C/EBPβ-2 is observed in the cytoplasmic compartments of the cells). The top right panel shows DNA staining with BoPro3 (red, staining of the nuclei). The bottom panel shows an overlay wherein essentially all nuclei contain yellow markings indicating a high level of C/EBPβ-2 expression.

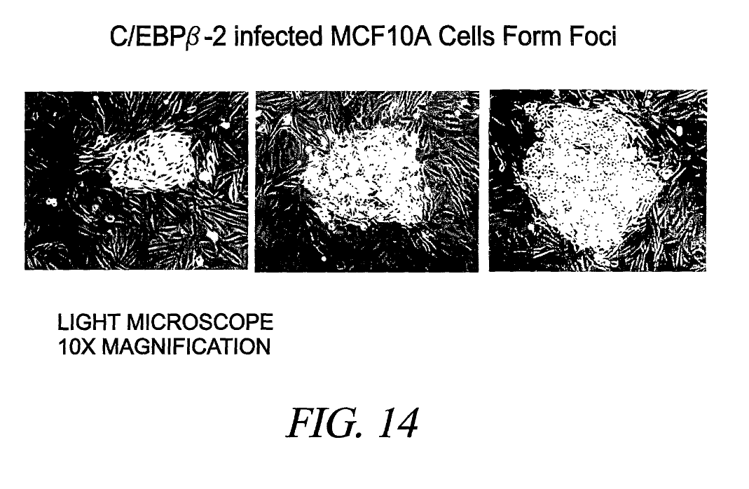

As shown in FIG. 14, cellular foci formed when MCF-10A cells were infected with pLZRS-his-C/EBPβ-2 or LZRS-his-C/EBPβ-2-IRES-eGFP retroviral vector (separate experiments, shown in the three panels). No such foci were formed from sham infections with LZRS IRES-eGFP retrovirus or from infection with LZRS his-C/EBPβ-1-IRES-eGFP retrovirus (data not shown). The formation of cellular foci is a model for transformation/tumorigenesis. These data demonstrate the transforming potential of C/EBPβ-2.

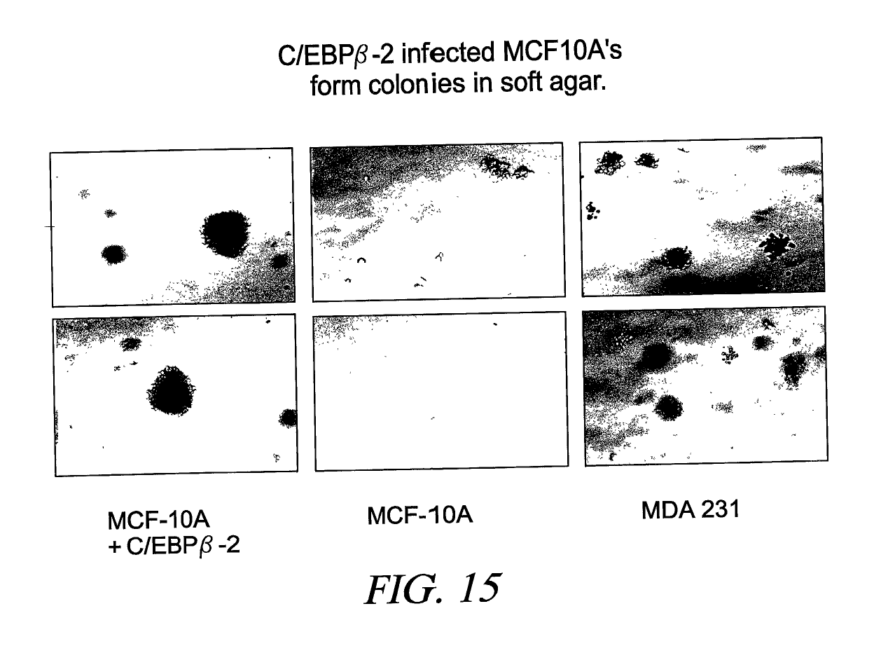

As shown in FIG. 15, MCF-10A cells infected with his C/EBPβ-2 retrovirus formed anchorage-independent growth colonies in soft agar. (left top and bottom panels, FIG. 15). The middle top and bottom panels of FIG. 15 are a control showing that uninfected MCF-10A cells did not form such colonies. MDA 231 cells which are known to form anchorage-independent growth colonies in soft agar and to be tumorigenic when transplanted into animals are shown in FIG. 15, right top and bottom panels. Anchorage-independent growth colony formation is an in vitro model for tumorigenesis.

In addition, the inventor has determined that C/EBPβ-2 activity stimulates the mitogenic factor cfos (Sealy et al., Mol Cell Biol (1997) 17(3):1744-1755, incorporated herein by reference). The inventor also determined that the mitogen Ras activates C/EBPβ-2, but not C/EBPβ-3 (Hanlon et al., J Biol Chem (1999) 274(20):14224-14228, incorporated herein by reference).

It is known that C/EBPβ regulates the expression of the β-casein and WAP genes during lactation (Seagroves et al. (1998) Genes Dev. 12(12)1917-1928, incorporated herein by reference).. It is contemplated in the present invention that an activity of the C/EBPβ-1 isoform is the transactivation of the β-casein and WAP genes, because the data above demonstrates that the C/EBPβ-1 isoform is a differentiation factor and C/EBPβ-2 is a proliferation factor (without being bound to mechanism).

In conclusion, C/EBPβ-2 promotes cellular proliferation. Under normal conditions, the regulated expression of C/EBPβ-2 and the biological activity of C/EBPβ-2 facilitates the proliferation of most cell types. The dysregulated expression and/or dysregulated biological activity of C/EBPβ-2, especially with regard to nuclear and particularly hyper-phosphorylated C/EBPβ-2 expression, results in a gain of mitogenic function and is tumorigenic. In fact, C/EBPβ-2 is observed in every proliferating cell type examined, including, but not limited to: epithelium, endothelium, lymphoid, and myeloid.

3.30 C/EBPβ-3 is an Anti-Tumor Agent: the Biological Basis

C/EBPβ-3 was known in the art to inhibit transactivation of a reporter gene by C/EBPβ-2 in cell culture (Descombes et al. (1991), supra). However, the biological activity of C/EBPβ-2 was not known and the role that C/EBPβ-2 played in tumorigenesis was also unknown in the prior art. In fact, it was believed that C/EBPβ-3 was a tumor promoter because apparent expression of C/EBPβ-3 was commonly observed in samples from tumor biopsies, infra.

It is disclosed in the present invention that C/EBPβ-3 is an anti-tumor agent. In certain embodiments, it is disclosed that the expression and/or a biological activity of C/EBPβ-3 is lost during tumorigenesis. The finding that C/EBPβ-3 is an anti-tumor factor is in contradiction to published literature. As reported in Rosen et al., (1998) Biochem Soc Symp (63)101-13, incorporated herein by reference; the expression of C/EBPβ-3 (referred to by Rosen et al., as LIP) is elevated in tumor derived cells. Similar results were reported by Zahnow et al., (1997) J Natl Cancer Inst 89(24):1887-91, incorporated herein by reference.

Rosen et al., (1998) noted that C/EBPβ-3 levels increase during proliferation and proposed that C/EBPβ-3 was a proliferation factor. Rosen et al., (1998) further proposed that C/EBPβ-3 was a tumorigenic factor. Zahnow et al., (1997) found the C/EBPβ-3 (referred to as LIP) expression was highly variable among 39 breast tumors sampled. C/EBPβ-3 expression was considered high in 9 of the 39 tumors and low or undetectable in the remaining 30 tumors. Zahnow et al., (1997) propose that C/EBPβ-3 is tumorigenic and should be evaluated as a prognostic marker for breast patients with breast cancer.

In the present invention; however, C/EBPβ-3 is disclosed to be an anti-tumor agent. The findings of Zahnow and Rosen may have arisen from proteolytic digestion of the C/EBPβ-2 isoform of C/EBPβ in their samples. It is also possible that in some tumors, C/EBPβ-3 is still expressed; but that an anti-tumor critical biological activity is lost which leads to tumorigenicity. The loss of C/EBPβ-3 activity can occur at the level of C/EBPβ-3 action itself or at a downstream point in a C/EBPβ-3 directed effector pathway. Overexpression of isolated C/EBPβ-3 (or introduction of C/EBPβ-3 polypeptide) can overcome such a defect by supplementing cellular C/EBPβ-3 levels, substituting for defective endogenous C/EBPβ-3, or possibly through an alternate or indirect pathway.

The expression of C/EBPβ-3 in HMECs and the panel of mammary carcinoma cell lines is shown in FIG. 6A. C/EBPβ-3 expression is exclusively in the nucleus of the HMECs (lane 2). The expression level of C/EBPβ-3 is greatly reduced in the panel of carcinoma cell lines with the most expression observed in MDA468 (lane 3), followed by MCF7-neo (lane 5), MCF7-218 (lane 6), BT474 (lane 9), and MDA 231 (lane 4). Essentially no C/EBPβ-3 expression is observed in the MCF7 (lane 7) or T47D (lane 8) carcinoma cell lines. C/EBPβ-3 was not expressed in highly differentiated cells (MDE, FIG. 6C) or non-proliferating, non-tumor cells from breast reduction surgery (FIG. 6B, lanes 2-4). Notably, C/EBPβ-3 expression was absent in ten out of ten primary breast tumor samples which (FIG. 6B, lanes 5-10, and additional data not shown).

Without being bound to mechanism, it is contemplated herein that non-proliferating cells do not necessarily express C/EBPβ-3 (FIG. 6B, lanes 2-4). However, proliferative stimuli, such as exogenous growth factors in the case of HMECs, leads to a nuclear expression of C/EBPβ-3 (FIG. 6A, lane 2). In tumor cells, the expression of C/EBPβ-3 is lost (FIG. 6B, lanes 5-10, and data not shown). Thus, C/EBPβ-3 is an anti-tumor factor. Without being bound to mechanism, it is contemplated that one mechanism of anti-tumor action by C/EBPβ-3 is through the inhibition of tumorigenic C/EBPβ-2 expression. It is also contemplated that C/EBPβ-3 acts by potentiating a programmed cell death of cells which begin inappropriate cell division. One possible way in which C/EBPβ-3 may act as a cell death factor is by directly or indirectly influencing Rb activity (see e.g., U.S. Pat. No. 5,912,236 to Xu et al., incorporated herein by reference).

3.31 Administration of C/EBPβ-3 Inhibits Colony Formation

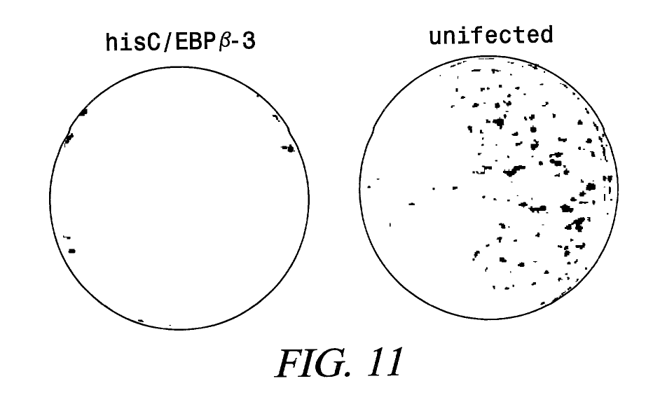

The anti-proliferation and/or the cell death promoting activities of exogenously introduced C/EBPβ-3 are demonstrated in colony forming assays, the results of which are shown in FIG. 11 (see, also, Example 10). A colony assay was performed with MDA 231 breast cancer cells transduced with LZRS-his-C/EBPβ-3 (see FIG. 11 and Example 10). Samples of MDA 231 cells (800 cells each) are transduced or sham-transduced with a retroviral-C/EBPβ-3 vector as described herein. Immunofluorescent staining indicates that approximately 80% of the MDA 231 cells are transduced by the LZRS-his-C/EBPβ-3 in this particular experiment (data not shown). The cells are plated in growth medium in culture. After one week in culture, colonies are easily visible upon hematoxylin staining if the cells survive and proliferate. The cells in the plate on the right were sham-transduced and the cells in the plate on the left received LZRS-his-C/EBPβ-3-C/EBPβ-3 vector (FIG. 11). Fewer than ten colonies are visible for the LZRS-his-C/EBPβ-3 transduced cells compared to a multitude of colonies on the sham-treated sample.

These data demonstrate that the introduction of exogenous C/EBPβ-3 into tumor cells inhibits growth and/or induces cell death in the MDA 231 cell population. Of further note is that cell proliferation is inhibited or cell death is stimulated in the 20% of tumor-derived cells that are not transduced by the LZRS-his-C/EBPβ-3 vector. Given an 80% transduction rate, it is expected that about 200 colonies out of 800 should form; however, fewer than ten colonies are identifiable. This is evidence that the “bystander effect” occurs during the course of treatment by the present invention. The bystander effect is where the biological activity of a treatment introduced into a subset of cells is effective on unmodified cells. The mechanism(s) of the bystander effect are not well understood, but empirically the existence of the bystander effect is well known (see e.g., Gene Therapy of Cancer (1999) Lattime et al., (eds.) Academic Press, especially pp:158-164 and 328, incorporated herein by reference). Thus, it is expected that the bystander effect will promote or enhance the treatment of tumors and tumorigenesis with C/EBPβ-1 and/or C/EBPβ-3, such treatments being described herein.

3.32 Administration of C/EBPβ-3 Inhibits Proliferation, Promotes Cell Death

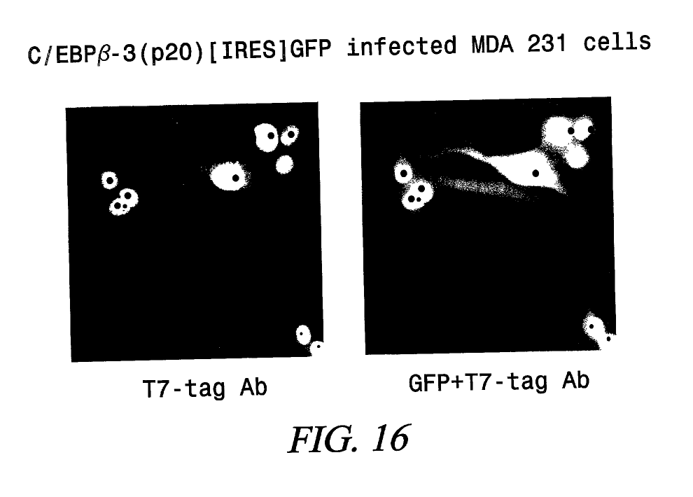

The LZRS-his-C/EBPβ-3-IRES-eGFP retrovirus was utilized for an in vitro model system for examining methods and results for treating tumors and tumorigenic cells with C/EBPβ-3. FIG. 16 shows that the LZRS-his-C/EBPβ-3-IRES-eGFP vector provided an excellent infection rate of tumor cells and expression C/EBPβ-3. The left panel of FIG. 16 shows T7-tag antibody staining of C/EBPβ-3 (p20) expression) which is highly localized to the nucleus of these cells. The right panel of FIG. 16 shows green fluorescent protein (GFP) and T7-tag antibody detection in the same field of cells. Essentially all cells in this particular field were infected and expressed C/EBPβ-3.

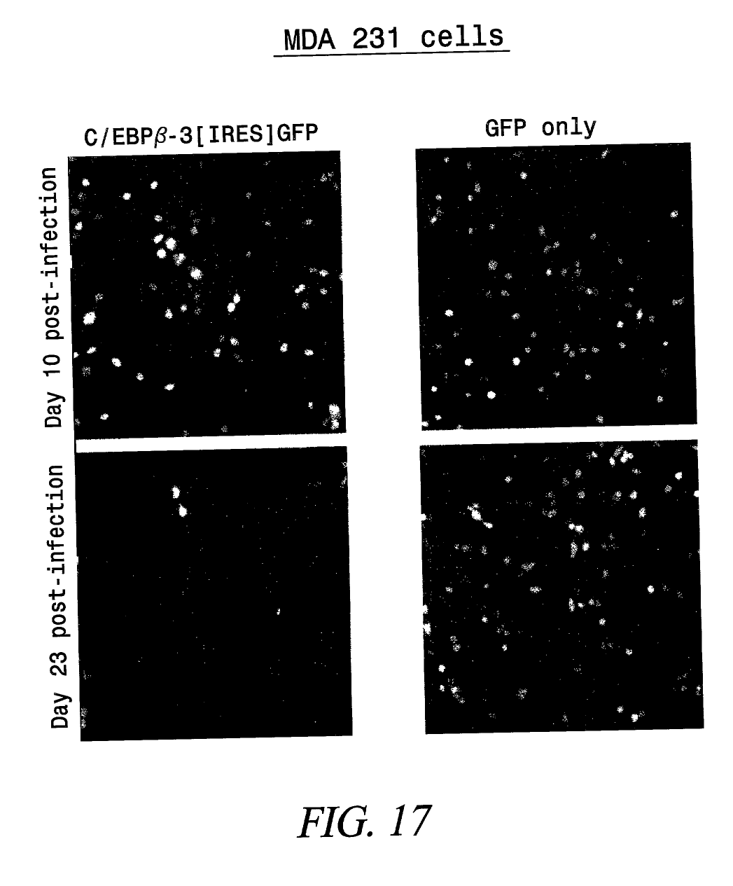

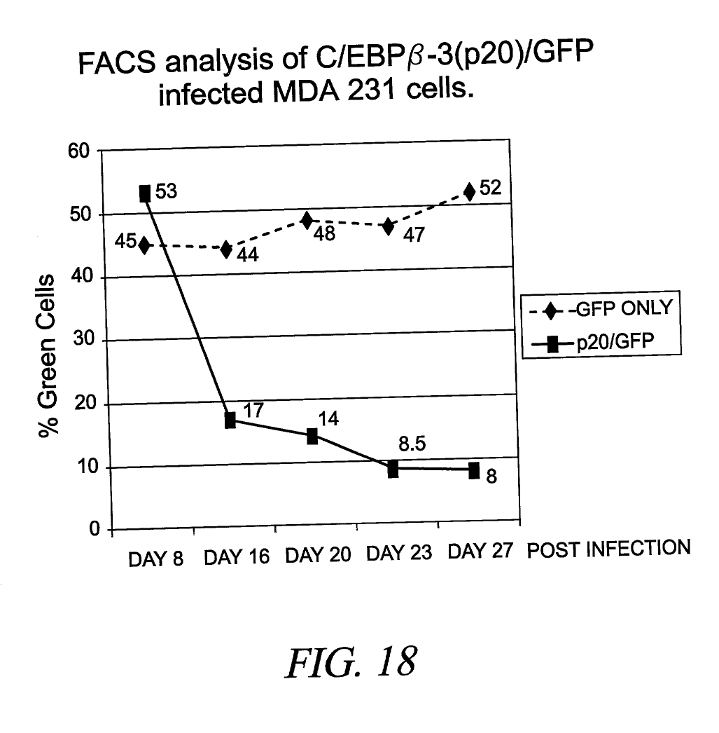

Treatment of tumors and tumorigenesis, the inhibition of cellular proliferation, and the promotion of cell death in a population of tumorigenic cells is demonstrated by the data shown in FIGS. 17 and 18. In FIG. 17, MDA 231 cells were either infected with LZRS-his-C/EBPβ-3-IRES-eGFP (left top and bottom panels) or sham infected with LZRS-IRES-eGFP retrovirus (right top and bottom panels). The top panels (left and right) are of cells examined at day 10 post-infection. The bottom panels (left and right) are of cells examined at day 23 post infection. The growth of the C/EBPβ-3 expressing MDA 231 cells was essentially stopped and a substantial number portion of the C/EBPβ-3 expressing cells died.

A quantitative FACS analysis of MDA 231 cells either infected with LZRS-his-C/EBPβ-3-IRES-eGFP (squares, solid line) or sham infected with LZRS-IRES-eGFP retrovirus (diamonds, dashed line) during a progression of days post-infection is shown in FIG. 18. These data demonstrate a substantial killing of C/EBPβ-3 expressing cells, while sham treated cells continued cell growth and were not subjected to promotion of cell death.

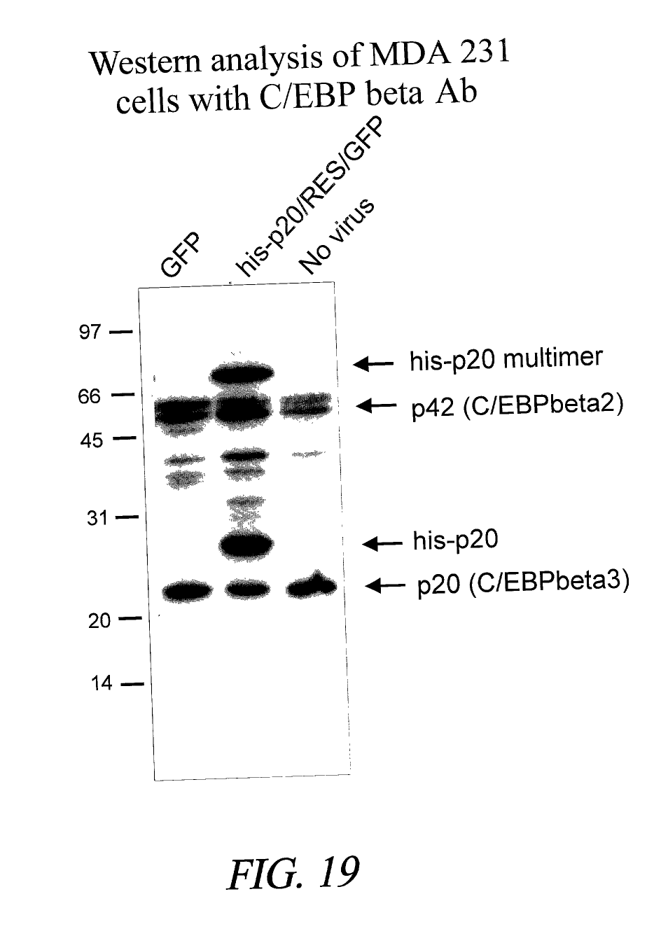

Western analysis of polypeptides in extracts from MDA 231 cells detected using the C-terminal C/EBPβ antibody demonstrates that the his-C/EBPβ-3 is expressed in high levels (FIG. 19). Lane 1 is an extract of cells sham infected with LZRS-IRES-eGFP retrovirus. Lane 2 is an extract of cells infected with LZRS-his-C/EBPβ-3-IRES-eGFP. Lane 3 is an extract of cells that were not infected. The relative positions are indicated for his-p20 (C/EBPβ-3) multimer, p42 (C/EBPβ-2), his-p20 (his-C/EBPβ-3), and p20 (C/EBPβ-3).

3.33 Administration of C/EBPβ-3 to erbB2 Expressing Mammary Cells

The receptor tyrosine kinase erbB2 (also known as neu and HER2) is overexpressed in greater than 25% of all human breast cancers and certain other cancers. Overexpression of erbB2 is a model for cancer formation and cells that overexpress erbB2 are a target for cancer treatments. The present inventor utilized an adenoviral based C/EBPβ-3 expression system (in this embodiment, pGEM-RecA-C/EBPβ-3) to express C/EBPβ-3 in BT474 cells which are an in vitro model system for erbB2 overexpressing mammary carcinoma. One round of infection with pGEM-RecA-C/EBPβ-3 resulted in an inhibition of cell growth and/or activation of cell death in greater than 90% of the treated cells within 5 days. The adeno-C/EBPβ-3 vector is constructed using standard techniques known in the art. For example, adenovirus type 5 in340 is recombined with the pGEM-RecA-C/EBPβ-3 in 293 cells and the adeno- C/EBPβ-3 is screened and selected for further experimentation and utilization herein (Hearing et al. (1983) Cell 33:695-703, incorporated herein by reference).

Given that C/EBPβ-3 acts to inhibit cell proliferation of tumor cells and tumorigenic cells, it is believed that administration of C/EBPβ-3 to most or all tumor cells will result in a significant treatment of the tumor, inhibition of tumorigenesis, inhibition of cell proliferation, and/or promotion of cell death. Also, none of these data exclude the possibility that there are other important targets for the C/EBPβ isoforms (C/EBPβ-1, C/EBPβ-2, and C/EBPβ-3) which need not be disclosed as the present invention does not depend upon mechanism or theory.

4.00 C/EBPβ Isoforms, Tumors, Tumorigenesis, and Treatment

The present invention discloses that C/EBPβ-1 is a cellular differentiation factor, inhibits proliferation, and that the loss of C/EBPβ-1 expression is tumorigenic, supra. The present invention discloses that C/EBPβ-2 is a cellular proliferation factor and that the gain of C/EBPβ-2 expression (particularly phosphorylated and nuclear expression of C/EBPβ-2) is tumorigenic, supra. The present invention discloses that C/EBPβ-3 is an inhibitor of cellular proliferation and in certain instances potentiates cell death, supra.

It is an aspect of the present invention that the introduction of C/EBPβ-1 to a proliferating cell induces differentiation and inhibits proliferation. It is another aspect of the present invention that the introduction of C/EBPβ-1 to a tumor cell inhibits the tumorigenic nature of the tumor cell. It is a further aspect of the present invention that the introduction of a C/EBPβ-1 isoform to a cell of a mammal treats a tumor in the mammal. It is still another aspect of the present invention that the introduction of C/EBPβ-1 to a population of cells inhibits proliferation and stimulates differentiation of the cells. One consequence of this is that the introduction of C/EBPβ-1 to a population of cells inhibits tumorigenesis of a cell within the population. Regardless of mechanism, the introduction of C/EBPβ-1 to a cell inhibits tumorigenesis. Regardless of mechanism, introduction of C/EBPβ-1 to a tumor cell diminishes capacity of the cell to metastasize. Regardless of mechanism, the introduction of C/EBPβ-1 to a tumor cell inhibits proliferation and stimulates differentiation of the tumor cell. Regardless of mechanism, the introduction of C/EBPβ-1 to a tumor cell leads to the destruction of the tumor cell.

In certain embodiments, the C/EBPβ-1 can be administered to a cell of the tumor itself to inhibit the proliferation and stimulate the differentiation of the tumor cell. In certain embodiments, the C/EBPβ-1 can be administered to cells in the area of the tumor to form a non-proliferative, differentiated cellular barrier to expansion of the tumor. In certain embodiments, the C/EBPβ-1 isoform can be administered to vascular cells in an area of a tumor to inhibit proliferation of the vascular cells and angiogenesis of the tumor. In certain embodiments, the C/EBPβ-1 can be administered to a population of cells in a mammal to inhibit a tumorigenic process from occurring in the population of cells. The inhibition of tumorigenesis would be especially desirable in the case of an individual that is predisposed to developing a cancer. One example is individuals with a mutation in a BRCA gene which results in a predisposition to the development of breast and ovarian cancer (see e.g., U.S. Pat. No. 5,891,857 to Holt et al., incorporated herein by reference). The introduction of C/EBPβ-1 to a population of cells in these individuals will maintain the cells in a differentiated state, inhibit proliferation, and inhibit tumorigenesis.

It is an aspect of the present invention that the introduction of C/EBPβ-3 to a proliferating cell inhibits proliferation and potentiates cell death. It is another aspect of the present invention that the introduction of C/EBPβ-3 to a tumor cell inhibits the tumorigenic nature of the tumor cell. It is a further aspect of the present invention that the introduction of a C/EBPβ-3 isoform to a cell of a mammal treats a tumor in the mammal. It is still another aspect of the present invention that the introduction of C/EBPβ-3 to a population of cells inhibits proliferation and potentiates cell death in the cells. One consequence of this is that the introduction of C/EBPβ-3 to a population of cells inhibits tumorigenesis of a cell within the population. Regardless of mechanism, the introduction of C/EBPβ-3 to a cell inhibits tumorigenesis. Regardless of mechanism, introduction of C/EBPβ-3 to a tumor cell diminishes capacity of the cell to metastasize. Regardless of mechanism, the introduction of C/EBPβ-3 to a tumor cell inhibits proliferation and potentiates cell death of the tumor cell. Regardless of mechanism, the introduction of C/EBPβ-3 to a tumor cell leads to the destruction of the tumor cell.

In certain embodiments, the C/EBPβ-3 can be administered to a cell of the tumor itself to inhibit the proliferation and stimulate the differentiation of the tumor cell. In certain embodiments, the C/EBPβ-3 can be administered to cells in the area of the tumor to form a non-proliferative, differentiated cellular barrier to expansion of the tumor. In certain embodiments, the C/EBPβ-3 isoform can be administered to vascular cells in an area of a tumor to inhibit proliferation of the vascular cells and angiogenesis of the tumor. In certain embodiments, the C/EBPβ-3 can be administered to a population of cells in a mammal to inhibit a tumorigenic process from occurring in the population of cells. The inhibition of tumorigenesis would be especially desirable in the case of an individual that is predisposed to developing a cancer. One example is individuals with a mutation in a BRCA gene which results in a predisposition to the development of breast and ovarian cancer (see e.g., U.S. Pat. No. 5,891,857 to Holt et al., incorporated herein by reference). The introduction of C/EBPβ-3 to a population of cells in these individuals will maintain the cells in a differentiated state, inhibit proliferation, and inhibit tumorigenesis. In certain embodiments, it is preferred that the activity level of introduced C/EBPβ-3 is greater than that of the C/EBPβ-2 already present in the tumor cell. Thus, in certain embodiments, it is desirable to produce a high C/EBPβ-3 to C/EBPβ-2 ratio through the introduction of C/EBPβ-3.

In certain exemplary embodiments of the above aspects and embodiments, C/EBPβ-1 and C/EBPβ-3 are co-administered. In embodiments wherein tumors or tumor cells are being treated it is not necessary to identify the cause(s) of tumorigenesis, whether genetic or otherwise. C/EBPβ-1 and C/EBPβ-3 action is fundamentally detrimental to the tumor or progression of tumorigenesis in a cell. These actions include, but are not limited to: the inhibition of proliferation, the stimulation of differentiation, and the potentiation of cell death. Likewise, it is not necessary to determine the nature of the tumorigenesis in embodiments wherein C/EBPβ-1 and/or C/EBPβ-3 are used to inhibit angiogenesis of a tumor.

5.00 Compositions and Methods of the Present Invention

The present invention provides compositions, including novel compositions, and methods for use thereof, as: anti-tumor agents, inhibitors of tumorigenesis, inhibitors of cellular proliferation, inducers of differentiation, and potentiators of programmed cell death.

Compositions provided for use in the present invention include the C/EBPβ-1 and the C/EBPβ-3 isoforms of C/EBPβ. Additional novel compositions are described herein. In certain preferred embodiments, a method of treating a tumor in a mammal in need thereof, includes administering an anti-tumor effective amount of a C/EBPβ isoform to the mammal. The isoforms of human C/EBPβ are diagramed in FIG. 1. As described supra, three isoforms of C/EBPβ correspond to three in-frame ATG translation initiation sites (including in human, mouse, and rat tissues). In human C/EBPβ these start sites are at approximately +1 (C/EBPβ-1), +70 (C/EBPβ-2), and +595 (C/EBPβ-3) in the human C/EBPβ nucleotide sequence (FIG. 1, SEQ ID NO:2). The present inventor has determined that C/EBPβ-1 and C/EBPβ-3 are anti-tumor agents and that C/EBPβ-2 is a tumor promoter. Thus, administering an anti-tumor amount of a C/EBPβ isoform applies to C/EBPβ-1 and C/EBPβ-3, but does not apply to C/EBPβ-2.

In certain embodiments, either C/EBPβ-1 or C/EBPβ-3 can be administered independently. In certain preferred embodiments C/EBPβ-1 and C/EBP-3 are administered in combination. Administration of C/EBPβ-1 and C/EBPβ-3 in combination means that the isoforms can be administered concurrently, or one before the other, or in multiple administrations wherein each administration can include C/EBPβ-1 or C/EBPβ-3 independently or concurrently. C/EBPβ-1 and C/EBPβ-3 may be administered in their protein form or encoded in a nucleic acid which expresses the isoforms (including being encoded by multiple nucleic acids). Thus, in certain embodiments, the use of the term “C/EBPβ-1 and C/EBPβ-3” is meant to include “C/EBPβ-1 and/or C/EBPβ-3” and grammatically modifying terms are to be understood as singular or plural as appropriate (is/are, segment/segments, etc.).

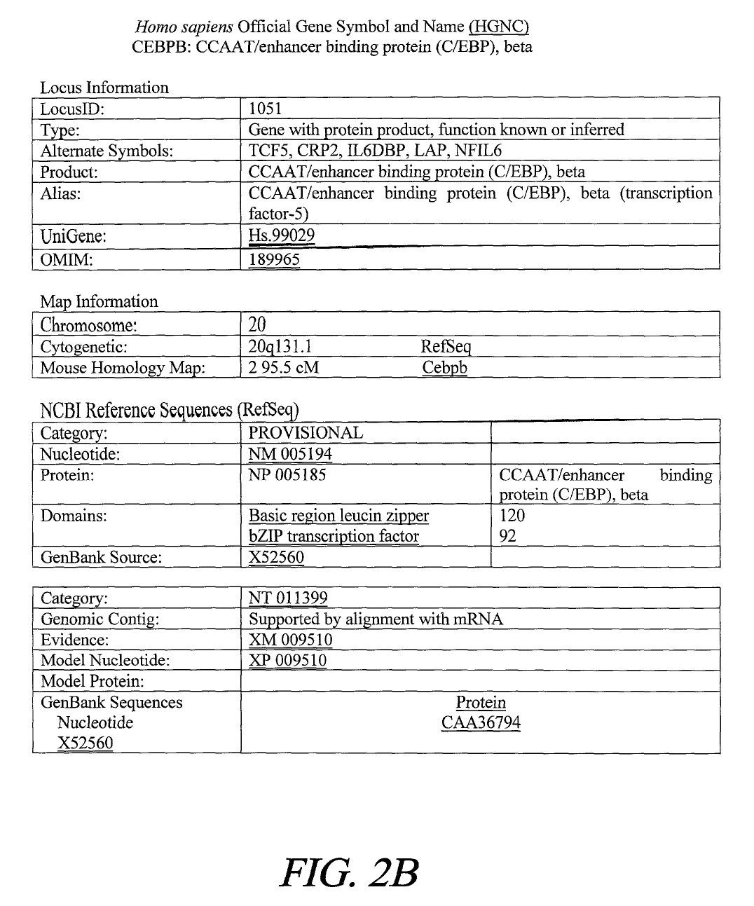

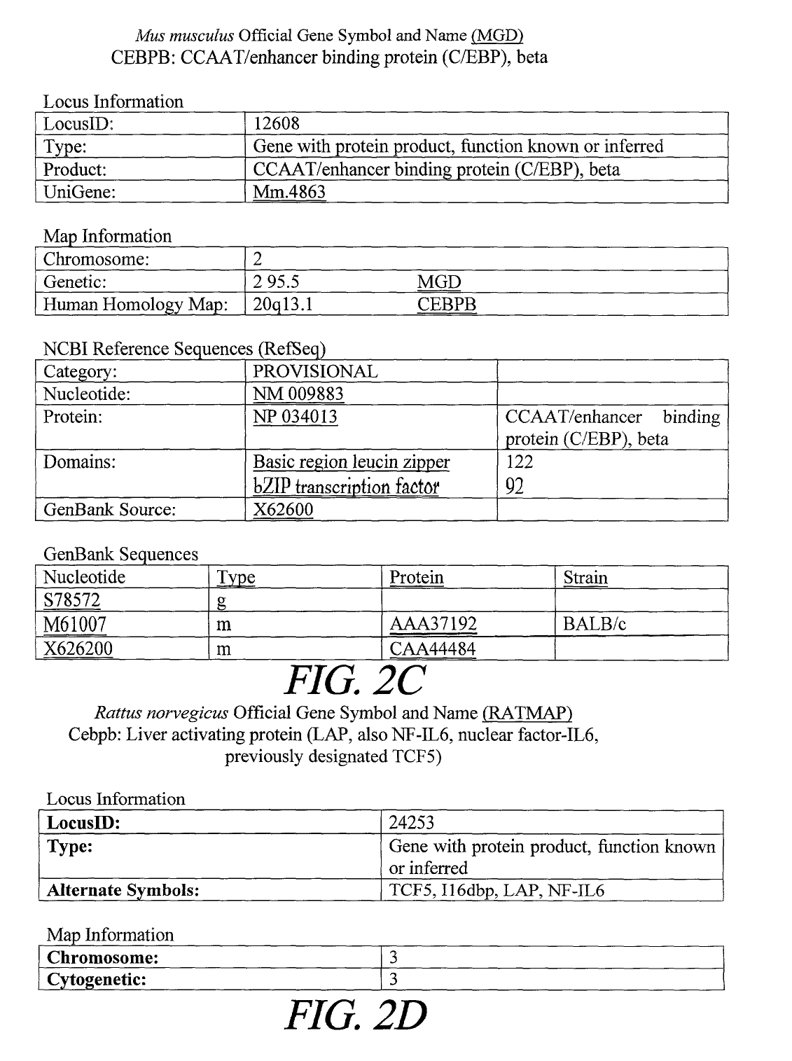

Preferred polynucleotide (SEQ ID NO:2) and polypeptide (SEQ ID NO:5) sequences of C/EBPβ-1 include the human reference sequences (FIGS. 2A and 2B, X52560 GenBank accession number). Preferred polynucleotide and polypeptide sequences of C/EBPβ-3 include (SEQ ID NO:4) and (SEQ ID NO:7), respectively. The terms “reference sequence” or “consensus sequence” are known in the art. The human CEBPB polynucleotide sequence is compiled from various cloned, sequenced, and analyzed fragments of genomic polynucleotide in the CEBPB gene of human cells.

Alternative polynucleotide sequences for C/EBPβ-1 include the mouse C/EBPβ-1, sequence (approximately positions 108 to 998 in SEQ ID NO:8) and the rat C/EBPβ-1 sequence (approximately positions 127 to 921 in SEQ ID NO:10). Alternative polynucleotide sequences for C/EBPβ-3 include the mouse C/EBPβ-3 sequence (approximately positions 560 to 998 in SEQ ID NO:8) and the rat C/EBPβ-3 sequence (approximately positions 498 to 921 in SEQ ID NO:10). Note, each sequence position indication includes the TAG stop codon.

Alternative polypeptide sequences for C/EBPβ-1 include the mouse C/EBPβ-1 sequence (SEQ ID NO:9) and the rat C/EBPβ-1 sequence (SEQ ID NO:18). Alternative polypeptide sequences for C/EBPβ-3 include the mouse C/EBPβ-3 sequence (approximately positions 152 to 296 in SEQ ID NO:9) and the rat C/EBPβ-3 sequence (approximately positions 153 to 297 in SEQ ID NO:18).

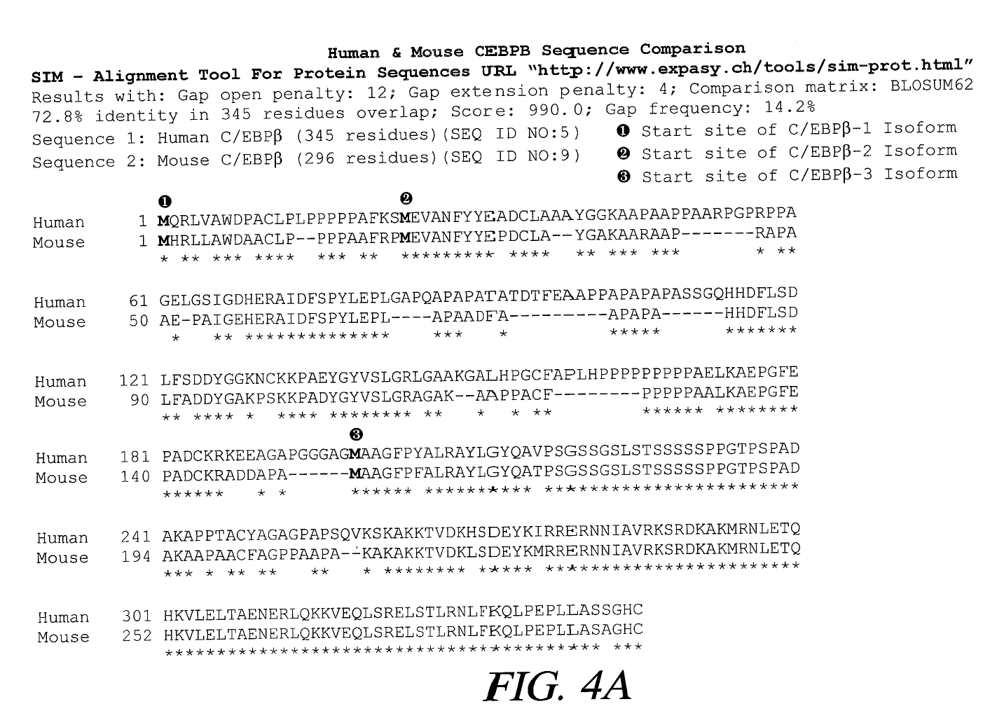

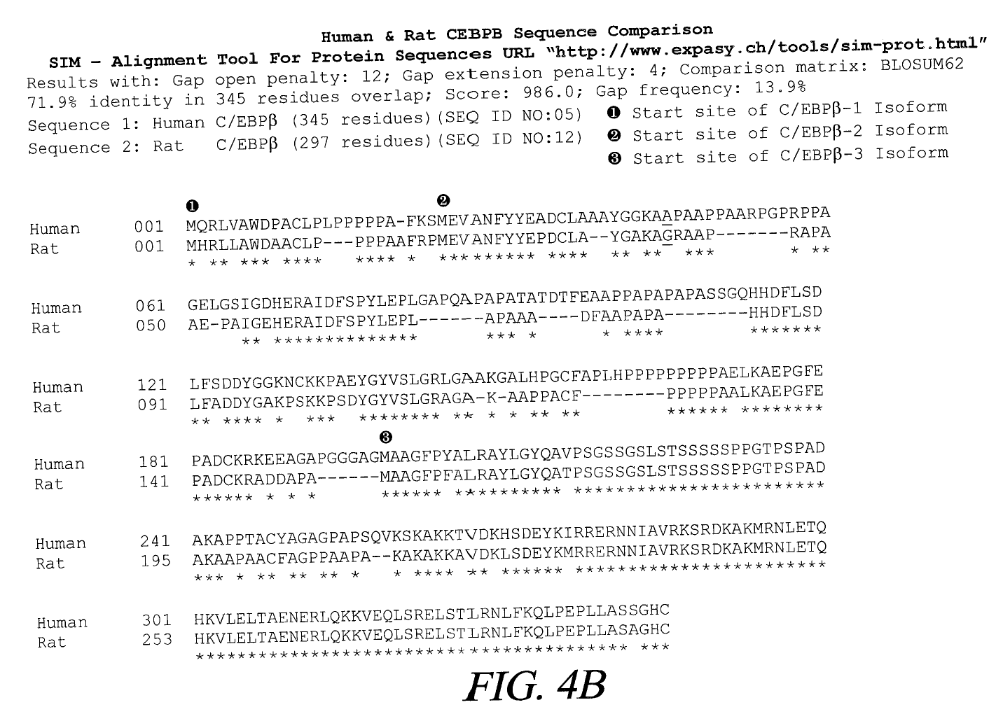

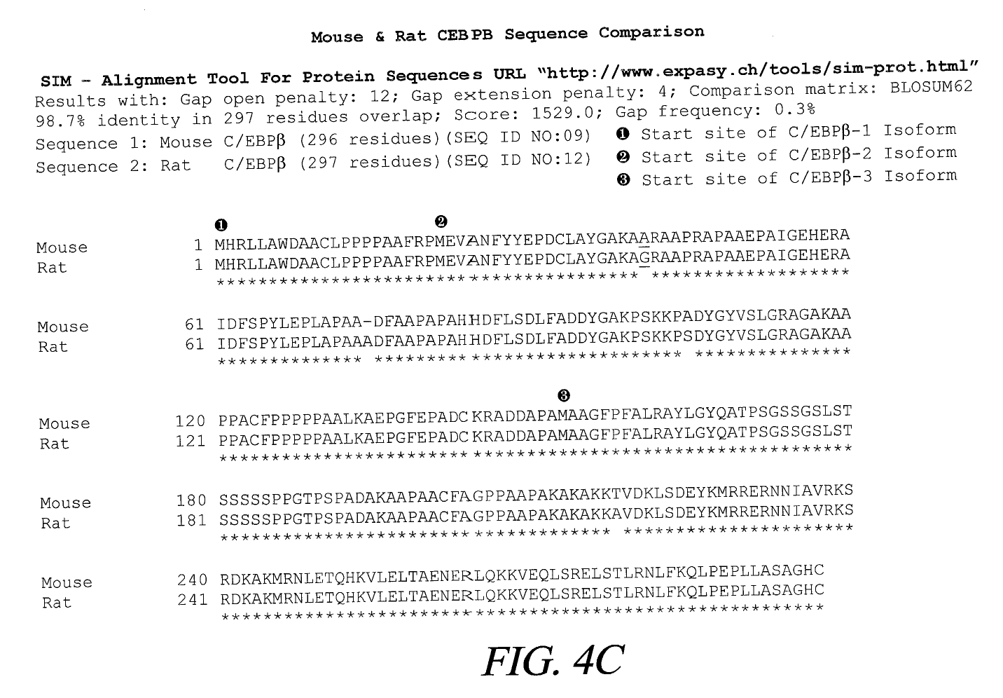

The relationships between each of the C/EBPβ isoforms in the polynucleotide and polypeptide sequences are annotated in FIGS. 3A-F and FIGS. 4A-C. C/EBPβ-1 and C/EBPβ-3 reference sequences can be found in public databases provided by the National Center for Biotechnology Information (NCBI) located at the United States National Library of Medicine (NLM). The NLM is physically located at 8600 Rockville Pike, Besthesda, Md. 20894; telephone: 301-594-5983. The NCBI is located on the world wide web at the URL “http://www.ncbi.nlm.nih.gov/” and the NLM is located on the world wide web at the URL “http://www.nlm.nih.gov/”. The NCBI website provides access to a number of scientific database resources including: GenBank, PubMed, Genomes, LocusLink, OMIM (Online Mendelian Inheritance in Man), Proteins, and Structures. A common interface to the polypeptide and polynucleotide databases is referred to as Entrez which can be accessed from the NCBI website on the World Wide Web at URL “http://www.ncbi.nlm.nih.gov/Entrez/” or through the LocusLink website.

FIG. 1 is a diagram of the genetic structure of the CEBPB gene and the relationship between C/EBPβ1, C/EBPβ-2, and C/EBPβ-3 isoforms. The basic structure of the C/EBPβ isoforms is similar in human, mouse, and rat tissues (FIGS. 4A-C). The genomic DNA for the CEBPB gene does not contain an intron; thus, the mRNA corresponds to the genomic sequence without interruption. It is believed that the expression products of the isoforms arise in the cell from a leaky ribosomal translation initiation of a single mRNA polynucleotide. However, the present invention is not bound by mechanism of production. Another possible mechanism is that RNA binding proteins may obscure a specific ATG site causing translation to start at the next available ATG site. It is also possible that the C/EBPβ-2 and/or C/EBPβ-3 protein isoforms arise in the cell through proteolytic cleavage of a longer polypeptide (e.g., C/EBPβ-1 or C/EBPβ-2, respectively); however, the inventor does not believe this mechanism to be correct or, at least, physiologically relevant. The mechanism is not important because it is shown herein that C/EBPβ-1 and C/EBPβ-3 function as anti-tumor agents. In addition, for a given population of cells, it is shown herein that C/EBPβ-1 and C/EBPβ-3 inhibit proliferation, stimulate differentiation, promote cell death, and act as anti-tumorigenesis agents.

5.01 Special Considerations for C/EBPβ-2 Expression

The inventor contemplates that expression of C/EBPβ-2 from exogenous nucleic acids used during the course of treatment would be detrimental to the outcome of treatment through the promotion of tumorigenesis. Therefore, in certain exemplary embodiments wherein polynucleotide sequences of C/EBPβ-1 are employed, the polynucleotide is modified to prevent a production of the C/EBPβ-2 isoform. For example, the polynucleotide segment is modified to prevent C/EBPβ-2 isoform production. Possible modes of C/EBPβ-2 production include, but are not limited to: translation initiation at the C/EBPβ-2 ATG translation initiation start site, proteolytic cleavage of a more encompassing polypeptide (e.g., a C/EBPβ-1 polypeptide), RNA processing, and blockage of other translation initiation start sites (e.g., the C/EBPβ-1 AUG).

The preferred method of preventing C/EBPβ-2 isoform production from a C/EBPβ polynucleotide is by using site-directed mutagenesis to eliminate the C/EBPβ-2 translation initiation start site (located in human CEBPB at approximately positions 368-370 of SEQ ID NO:1, see FIG. 3A). It is also preferred that alternative sequences of C/EBPβ-1 such as mouse and rat C/EBPβ-1 are modified to eliminate the C/EBPβ-2 translation initiation start site. As a result of the elimination of the C/EBPβ-2 translation start site, C/EBPβ-1 polynucleotides are not able to, or do not, express C/EBPβ-2 in the treated cells.

It is also preferred to modify recombinant C/EBPβ-1 polynucleotides used for pharmaceutical production of C/EBPβ-1 in host cells to prevent C/EBPβ-2 expression. This reduces the initial amount of C/EBPβ-2 in such preparations. By using host cells that do not express C/EBPβ-2 it is therefore possible to produce preparations of C/EBPβ-1 and/or C/EBPβ-3 that are essentially free of C/EBPβ-2 without the need of separating isolating C/EBPβ-1 and/or C/EBPβ-3 away from C/EBPβ-2.

If proteolytic digestion of C/EBPβ-1 to C/EBPβ-2 is found to occur in a particular system, it is preferred that the protease binding site is mutated to inhibit or prevent proteolysis. This is not expected to arise, however. Otherwise, the C/EBPβ-1 can be combined with a protease inhibitor, protein stabilizing agent, or stored under protein stabilizing conditions (i.e., refrigeration, freezing, desiccated, etc.).

5.02 Special Considerations for C/EBPβ-1 Expression

In certain exemplary embodiments wherein polynucleotide sequences of C/EBPβ-1 are employed, the polynucleotide is modified to enhance a production of the C/EBPβ-1 isoform. For example, a polynucleotide segment including C/EBPβ-1 is mutated through site-directed mutagenesis to create a Kozak sequence at the C/EBPβ-1 translation initiation site (approximately positions 299 to 301 in SEQ ID NO:1). The use of a Kozak sequence to enhance expression of a gene is well known in the art. In the present invention, the Kozak sequence is used to selectively enhance the expression of one C/EBPβ isoform over another For example, C/EBPβ-1 expression is enhanced over C/EBPβ-2 expression through the use of a Kozak sequence at the C/EBPβ-1 start site.

5.03 Compositions and Combinations: C/EBPβ-1 and C/EBPβ-2 Expression

In certain highly exemplary embodiments regarding the expression of C/EBPβ-1 from a polynucleotide segment, a novel segment is created through modifications to both prevent expression of the C/EBPβ-2 isoform and to enhance the expression of the C/EBPβ-1 isoform. In certain embodiments, multiple mutations are made in the segment to eliminate the translation start site for C/EBPβ-2 and to create a Kozak sequence at the C/EBPβ-1 translation start site.

A novel composition of the present invention comprises a polynucleotide encoding a C/EBPβ-1 isoform having a modification which eliminates a C/EBPβ-2 production site. In certain embodiments, the C/EBPβ-2 translation initiation site is modified through site-specific mutagenesis. (Translation start site means the same as translation initiation site and these terms are known in the art.) Another novel composition of the present invention comprises a polynucleotide segment encoding a C/EBPβ-1 isoform having a modification which creates a Kozak sequence at a C/EBPβ-1 translation initiation site. Still another novel composition of the present invention comprises a polynucleotide segment having a modification which eliminates a C/EBPβ-2 translation start site and creates a Kozak sequence at a C/EBPβ-1 translation start site. This novel polynucleotide segment is called a C/EBPβ-1 expression enhanced segment or a C/EBPβ-1 expression enhanced polynucleotide segment. A polynucleotide sequence of a C/EBPβ-1 expression enhanced segment is set forth in SEQ ID NO:17.

Naturally, expressed polypeptides, expressed proteins, vectors containing the segment, and host cells are within the spirit and scope of the present invention. Also, conservatively modified variants and biologically functional equivalents of a C/EBPβ-1 expression enhanced segment can be made which are within the spirit and scope of the present invention.

In certain embodiments, a C/EBPβ-1 expression enhanced polynucleotide segment may be linked to an expression vector. It is preferred that the expression vector is operably linked to the C/EBPβ-1 expression enhanced polynucleotide segment. An expression vector combined with a C/EBPβ-1 expression enhanced segment is called a C/EBPβ-1 expression enhanced vector. It is specifically contemplated that, in certain embodiments, C/EBPβ-3 may be expressed from a C/EBPβ-1 expression enhanced segment. This includes the expression of C/EBPβ-3 from the internal translation start site for C/EBPβ-3 within a C/EBPβ-1 polynucleotide and the inclusion of a C/EBPβ-1 enhanced segment plus a C/EBPβ-3 segment in a vector. Thus, one polynucleotide sequence can be engineered to express both C/EBPβ-1 and C/EBPβ-3, but not C/EBPβ-2.

5.04 Special Considerations for Nuclear Import

It is believed by the inventor that the protein and nucleic acid compositions of the present invention act in the nuclear compartment of the cell (without being bound to mechanism). Therefore, in certain preferred embodiments, compositions including, but not limited to, sequences of C/EBPβ-1 and C/EBPβ-3, contain a nuclear localization sequence (NLS) to facilitate nuclear import (either peptide or encoding nucleic acid). C/EBPβ contains two putative NLSs as determined by PSORT II an online sequence analysis tool available at URL http://psort.nibb.ac.jp:8800/coded for by Dr. Kenta Nakai and physically located at the Human Genome Center, Institute for Medical Science, University of Tokyo, 4-6-1 Shirokane-dai, Minato-ku, Tokyo 108-8639, Japan (e-mail: knakai@ims.u-tokyo.ac.jp). The program manual and results of a PSORT II analysis for human C/EBPβ are provided infra (see Appendix A) and each reference listed is hereby incorporated herein by reference.

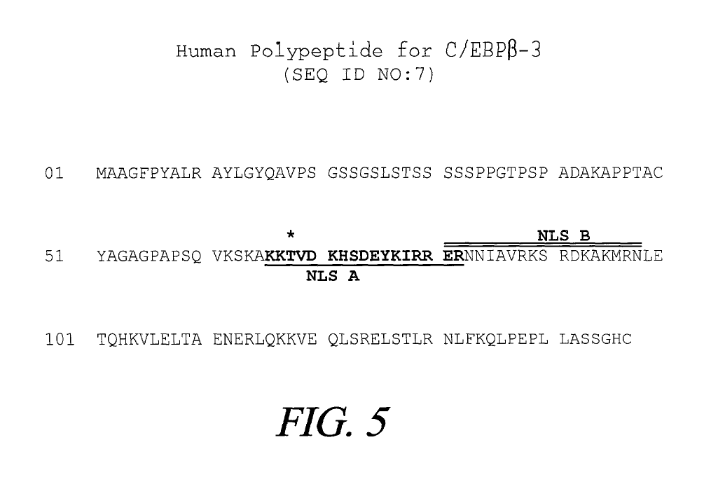

The results of the PSORT II analysis tool predict that KKTVDKHSDEYKIRR (SEQ ID NO:15 and NLS A in FIG. 5) and RRERNNIAVRKSRDKAK (SEQ ID NO:16 and NLS B in FIG. 5) are nuclear localization sequences. Both of these sequences are located in the C-terminal portion of C/EBPβ reference sequence and are contained within both C/EBPβ-1 and C/EBPβ-3. In certain embodiments of the present invention, any NLS may function to facilitate nuclear import of a composition of the present invention (see e.g., Stochaj et al. (1993) J of Cell Science 104:89-95, incorporated herein by reference). A preferred NLS is RRERNNIAVRKSRDKAK (SEQ ID NO:16) and certain preferred polypeptide sequences (or encoding nucleic acids) will include this peptide sequence (or encoding nucleic acid sequence). An even more preferred NLS is KKTVDKHSDEYKIRR (SEQ ID NO:15) and certain even more preferred polypeptide sequences (or encoding nucleic acids) will include this peptide sequence (or encoding nucleic acid sequence). In certain embodiments, preferred sequuences will include both NLS A and NLS B (FIG. 5). The threonine with an “*” over it in FIG. 5 and at approximately position 68 (SEQ ID NO:7) is a potential phosphorylation site that the inventor believes may enhance C/EBPβ-1 and/or C/EBPβ-3 activity. In certain preferred embodiments, polypeptides (or encoding nucleic acids) will include this threonine.

5.10 Biological Functional Equivalents

Preferred and alternative polynucleotide and polypeptide sequences useful for embodiments of the present invention are provided herein. Thus, it is not necessary to identify additional polynucleotide or polypeptide sequences to practice the present invention. However, as is known to one with skill in the art, the biological function or activity of a gene product may not correspond directly to an absolute polynucleotide or polypeptide sequence of the gene product. Therefore, the inventor specifically contemplates that alterations to sequences provided herein, including in the Sequence Listings of this Specification, may be made or used wherein the altered sequences, or methods of use thereof, are equivalent to sequences, or methods of use thereof, and are within the spirit and scope of the present invention. These equivalent sequences are referred to as biologically functional equivalents, or simply as functional equivalents. Functional equivalents can include, but are not limited to: conservatively modified variants, degeneracy of the nucleic acid code, polymorphisms, certain insertions and deletions, and certain length variants. Methods for altering sequence residues and testing the altered sequences for function or activity are known in the art or described herein. These alterations may be natural or made by the “hand of man”.

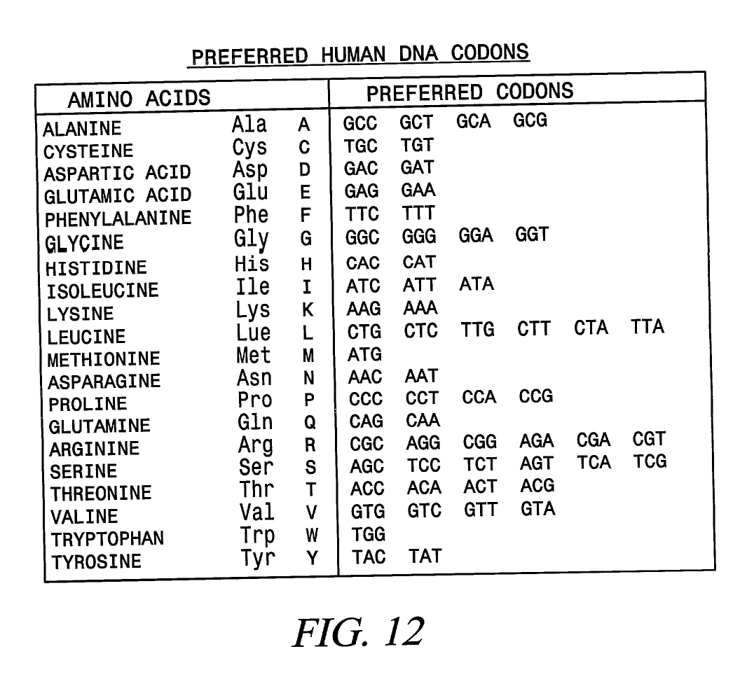

At the nucleotide level, different codons can encode the same amino acid. In other words, the genetic code is degenerate (Alberts et al., Molecular Biology of the Cell, (1989) 2nd Edition, Garland Publishing, Inc., and incorporated herein by reference). The terms “wobble” and “nucleic acid degeneracy” are used herein to refer to codons that encode the same amino acid, such as the six codons for arginine or serine. FIG. 12 lists the preferred human codons. The codons are listed in decreasing order of preference from left to right in the table (Wada et al. (1990) Nuc. Acids. Res., 18:2367-2411, included herein by reference). Codon preferences for other organisms also are well known to those of skill in the art (Wada et al., 1990, supra). Thus, one with skill in the art knows that two different polynucleotides can encode identical polypeptide sequences due to codon wobble.

It is understood in the art that amino acid and nucleic acid sequences may include additional residues, such as additional N-terminal or C-terminal amino acids or 5′ or 3′ sequences, and yet still be essentially as set forth in one of the sequences disclosed herein; so long as the sequence meets the criteria set forth herein, including the maintenance of at least one biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes between coding regions (Alberts et al., supra, incorporated herein by reference). Thus; about 1, 2, 3, 4, 5, 6, 7, or more than 7 amino acids could be added to a polypeptide and the polypeptide may still retain at least one biological activity. Or; about 1, 2, 3, 4, 5, 6, 7, or more than 7 nucleotides could be added to a polynucleotide and expression products of the polynucleotide may still retain at least one biological activity.

It also is understood in the art that amino acid and nucleic acid residues may be removed from the N-terminal or C-terminal ends of polypeptide or 5′ or 3′ ends of polynucleotide sequences, and yet still be essentially as set forth in one of the sequences disclosed herein; so long as the sequence meets the criteria set forth herein, including the maintenance of at least one biological protein activity where protein expression is concerned. The removal of terminal sequences particularly applies to nucleic acid sequences that may, for example, include various non-coding sequences flanking either of the 5′ or 3′ portions of the coding region or may include various internal sequences, i.e., introns, which are known to occur within genes between coding regions (Alberts et al., supra, incorporated herein by reference). Thus; about 1, 2, 3, 4, 5, 6, 7, or more than 7 amino acids could be removed from a polypeptide and the polypeptide may still retain at least one biological activity. Or, about 1, 2, 3, 4, 5, 6, 7, or more than 7 nucleotides could be removed from a polynucleotide and expression products of the polynucleotide may still retain at least one biological activity.

C/EBPβ does not contain an intron as found in various organisms including, but not limited to: human, mouse, and rat. However, if desired, it is possible using techniques known to one with skill in the art, to include an intron in a recombinant C/EBPβ polynucleotide sequence. For example, a bovine growth hormone (bGH) intron including splice sites may be added. In certain instances, the addition of an intron to a recombinant polynucleotide has been observed to increase expression of the encoded expression product in eukaryotic cells. It is understood that the addition of an intron creates a functionally equivalent sequence.

With regard to the removal of sequences, a primary difference between C/EBPβ-1 and C/EBPβ-2 is the segment at the N-terminal (protein) and 5′ end (nucleic acid) that is found in C/EBPβ-1, but not in C/EBPβ-2. For example, in human cells, there is a 23 amino acid or a 69 basepair sequence (approximate lengths, SEQ ID NO:5 and SEQ ID NO:2) at the N-terminus or 5′ end of C/EBPβ-1 which is absent in C/EBPβ-2 (FIGS. 1, 3A, and 3B). It is contemplated that removal of several amino acids or nucleotides may yield a sequence essentially as set forth in SEQ ID NO:2 or SEQ ID NO:5, respectively. However removal of residues from C/EBPβ-1 may yield a sequence that loses at least one of the beneficial activities of C/EBPβ-1 and C/EBPβ-3 and gains at least one of the detrimental activities of C/EBPβ-2 as disclosed herein. Tests are provided herein to enable one of ordinary skill in the art to determine if a sequence is equivalent to one of the sequences listed in the Sequence Listings. C/EBPβ-2 and C/EBPβ-2 equivalents should not be used for treatment of tumorigenesis in a mammal.

It is further understood in the art that insertions and deletions may be made within the amino acid and nucleic acid sequence, and yet still be essentially as set forth in one of the sequences disclosed herein; so long as the sequence meets the criteria set forth herein, including the maintenance of biological protein activity where protein expression is concerned. In general, insertions or deletion of residues in the coding region of a listed nucleic acid encoding a C/EBPβ-1 and/or C/EBPβ-3 protein should be made such that the net insertion or deletion is a multiple of 3. Thus, it is preferred that the reading frame of the polynucleotide sequence be maintained, as is known in the art (Alberts et al., supra, incorporated herein by reference).

Excepting intronic or flanking regions, and allowing for the degeneracy of the genetic code, sequences that have between about 70% and about 79%; or more preferably, between about 80% and about 89%; or even more preferably, between about 90% and about 99% or more; of nucleotides that are identical to the nucleotides shown in the sequences of SEQ ID NO:1-4, 8,10-11,15-17, and 21-24 will be sequences that are “essentially as set forth in SEQ ID NO:1-4, 8,10-11,15-17, and 21-24”. Sequences that are essentially the same as those set forth in SEQ ID NO:1-4, 8,10-11,15-17, and 21-24 also may be functionally defined as sequences that are capable of hybridizing to a nucleic acid segment containing the complement of SEQ ID NO:1-4, 8,10-11,15-17, and 21-24 under high stringency conditions as defined herein. In certain embodiments, sequences that are considered “essentially as set forth” in a sequence listed herein are also biologically functional equivalents to the listed sequence if at least one biological activity is found in common. However, sequences that share homology with C/EBPβ-1 and/or C/EBPβ-3 and appear to be essentially as set forth in a C/EBPβ-1 and/or a C/EBPβ-3 sequence by sequence homology, but that impart a biological activity of C/EBPβ-2 (e.g., tumor promotion, growth promotion, stimulation of proliferation, or inhibition of differentiation); are specifically understood not to be functional equivalents of a C/EBPβ-1 and/or C/EBPβ-3 sequence or variant thereof.

At the protein level, peptide sequences that are essentially the same, in general, are capable of cross-reacting with antibody raised against the respective peptide factor. In certain embodiments, polypeptides that are essentially the same as determined by antibody cross-reactivity, may be biological equivalents of polypeptides listed herein including C/EBPβ-1 (SEQ ID NO:5) and C/EBPβ-3 (SEQ ID NO:7). However, in the case of C/EBPβ, an antibody raised against an epitope that is common to two or more isoforms will then cross-react with each isoform containing the common epitope. Thus, in certain embodiments, it is important to determine that the polypeptide under consider is a functional equivalent to C/EBPβ-1 and/or C/EBPβ-3 actually has a biological activity of C/EBPβ-1 and/or C/EBPβ-3, but that it does not have a biological activity of C/EBPβ-2 (e.g., tumor promotion, growth promotion, stimulation of proliferation, or inhibition of differentiation). Methods for isolating, resolving, and analyzing protein/antibody interactions are well known in the art including techniques such as SDS-PAGE and Western analysis. Using SDS-PAGE and Western analysis in conjunction with the N-terminal antibody identified herein to C/EBPβ-1 and the C-terminal C/EBPβantibody (see FIG. 1), one with skill in the art can resolve and identify all three C/EBPβ isoforms (C/EBPβ-1, C/EBPβ-2, and C/EBPβ-3) from biological samples through observation of molecular weight and reaction with the antibodies.

Naturally, the present invention also encompasses DNA segments that are complementary, or essentially complementary, to the sequences set forth in SEQ ID NO:1-4, 8,10-11,15-17, and 21-24. Nucleic acid sequences that are “complementary” include those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO:1-4, 8,10-11,15-17, and 21-24 under high stringency conditions.

The nucleic acid segments of the present invention, regardless of the length of the coding sequence itself, may be combined with other DNA sequences (one or more of each), such as promoters, polyadenylation signals, additional restriction enzyme sites, multiple cloning sites, internal ribosome entry sites, introns, other coding segments, membrane transport sequences, and the like, such that their overall length may vary considerably. It is therefore contemplated that a nucleic acid fragment of almost any length may be employed, with the total length preferably being limited by the ease of preparation and use in the intended recombinant DNA protocol. Therefore, the terms “C/EBPβ-1 gene” and “C/EBPβ-3 gene” (also referred to as, for example, C/EBPβ-1 polynucleotide, C/EBPβ-1 sequence, or simply as C/EBPβ-1, or C/EBPβ-3) may also comprise any combination of associated control sequences. Furthermore, those skilled in the art of mutagenesis will appreciate that other analogs, as yet undisclosed or undiscovered, may be used to construct C/EBPβ-1 analogs (mutants, variants, etc). Additional meaning of biological functional equivalents, similarity, percent similarity, equivalents, substantially identical sequences, essentially the same, and essentially similar sequences and activities are described in U.S. Pat. No. 5,922,688 to Hung et al., incorporated herein by reference.

Naturally, the present invention also encompasses peptides and polypeptides (or the nucleic acid sequences that encode such peptides and polypeptides) that contain conservatively modified variants of the sequences listed in the Sequence Listings. One with skill in the art is able to determine conservative sequence modifications. In the case of a polypeptide, amino acid substitutions, such as those which might be employed in modifying CEBPB are generally based on the relative similarity of the amino acid side-chain substituents, for example, their hydrophobicity, hydrophilicity, charge, size, and the like.

An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as conservative amino acid changes or substitutions. In general, conservatively modified variants of a sequence may include one or more conservative amino acid change or substitution.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte et al., J. Mol. Biol. (1982) 157(1):105-32, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It also is understood in the art that the substitution of like amino acids can be made effectively on the basis of hydrophilicity. U.S. Pat. No. 4,554,101 to Hopp, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, supra, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

While discussion has focused on conservatively modified variant polypeptides and functionally equivalent polypeptides arising from amino acid changes, it will be appreciated that these changes may be effected by alteration of the encoding polynucleotide; taking into consideration also that the genetic code is degenerate and that two or more codons may code for the same amino acid.

5.20 Sequence Modification Techniques

Modifications to C/EBPβ-1 and C/EBPβ-3 sequences may be made during chemical synthesis of the polymers (either nucleotide or peptide synthesis). It is believed, however, that site-directed mutagenesis of an encoding nucleic acid, (e.g., human CEBPB, as set forth in SEQ ID NO:1) creating a suitably altered polynucleotide sequence is the most cost effective method of generating an altered polynucleotide sequence of C/EBPβ-1 and C/EBPβ-3. Where the C/EBPβ-1 and C/EBPβ-3 protein is desired, then the mutated sequence is expressed including in culture (in vitro or ex vivo) or in vivo.

Site-directed mutagenesis is a technique useful in the preparation of individual peptides, or biologically functional equivalent proteins or peptides, through specific mutagenesis of the underlying DNA. Several methods for site directed mutagenesis are described in U.S. Pat. No. 4,873,192 to Kunkel, incorporated herein by reference and in U.S. Pat. No. 4,351,901 to Ball, incorporated herein by reference. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 14 to about 25 nucleotides in length is preferred, with about 5 to 10 residues on both sides of the junction of the sequence being altered. The primers can be selected by one with ordinary skill in the art based upon information provided herein, including the Sequence Listings and Figures.

The technique of site-specific mutagenesis is well known in the art as exemplified by publications (Adelman et al., (1983) DNA 2(3)183-193, incorporated herein by reference). As will be appreciated, the technique typically employs a phage vector which exists in both a single stranded and double stranded form. Typical vectors useful in site-directed mutagenesis include vectors such as the M13 phage. These phage are readily commercially available and their use is well known to those skilled in the art. Double stranded plasmids are also routinely employed in site directed mutagenesis which eliminates the step of transferring the gene of interest from a plasmid to a phage. Kits for phage based site directed mutagenesis are commercially available. In addition PCR based methods which may, or may not, involve phage are known in the art and kits for such purposes are commercially available.

In certain known techniques, site-directed mutagenesis is performed by first obtaining a single-stranded vector or melting apart the two strands of a double stranded vector which includes within its sequence a DNA sequence which encodes the desired CEBPB isoform. An oligonucleotide primer bearing the desired mutated sequence is prepared, generally synthetically as is known to one of ordinary skill in the art. This primer is then annealed with the single-stranded vector, and subjected to DNA polymerizing enzymes such as Escherichia coli (E. coli) polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement. Various selection methods that increase the percentage of specifically modified clones over wild-type are known and available commercially.

Kalderon et al. (1984) report several mutagenic methods which have proved useful in mutating the native LT gene. Specifically, Kalderon et al. teach deletion mutations by displacement-loop mutagenesis and by the random insertion of Eco RI linkers into the LT gene. Further, point mutation by deletion-loop mutagenesis is taught. The reference also teaches screening procedures for determining the success of such mutations. The teachings of Kalderon et al. (1984) Virology 139(1)109-137 are incorporated herein by reference.

The preparation of sequence variants of the selected gene using site-directed mutagenesis is provided as a method of producing potentially useful C/EBPβ-1 and C/EBPβ-3 and is not meant to be limiting as there are other ways in which sequence variants of these nucleotide and peptides may be obtained. For example, recombinant vectors encoding the desired genes may be treated with mutagenic agents to obtain sequence variants for the mutagenesis of plasmid DNA using hydroxylamine or random mutagenesis may be performed using the PCR technique.

The preferred method of carrying out site-directed mutagenesis, for certain embodiments, is described in the Examples section. Briefly, a double-stranded plasmid including a segment encoding the C/EBPβ-1 and/or C/EBPβ-3 sequence of interest is restricted with a restriction endonuclease(s) on either side of the desired site of the mutation. Synthetic oligo nucleotides (with at least a portion of each oligo being complementary) containing the site-directed mutation are annealed, then reacted with the restricted plasmid, and ligated. This forms plasmids including the encoding segment of interest carrying the site-specific mutation(s) desired. The pool of plasmids are screened and sequenced using known methods to identify an acceptable clone.

Sequence analysis of a potentially mutant nucleic acid sequence is carried out by methods known in the art, typically by either Sanger dideoxy sequencing (Sanger et al., PNAS (1977) 74:5363-5467, incorporated herein by reference; U.S. Pat. No. 4,871,929 to Barnes; and U.S. Pat. No. 4,962,020 to Tabor et al., each patent incorporated herein by reference) or automated sequencing (U.S. Pat. No. 5,365,455 to Tibbetts et al., incorporated herein by reference).

In addition to the CEBPB peptidyl compounds described herein, the inventor also contemplates that other sterically similar compounds may be formulated to mimic the key portions of the peptide structure. Such compounds may be used in the same manner as the peptides of the invention and hence are also functional equivalents. The generation of a structural functional equivalent may be achieved by the techniques of modeling and chemical design known to those of skill in the art. It will be understood that all such sterically similar constructs fall within the scope of the present invention.

In addition to sequence equivalents, the inventor also specifically contemplates further biological variations of C/EBPβ-1 and C/EBPβ-3 that are functionally equivalent to C/EBPβ-1 and C/EBPβ-3. For example, C/EBPβ-1 and C/EBPβ-3 may be altered by phosphorylation, glycosylation, or other biological modification. The expression of C/EBPβ-1 and C/EBPβ-3 in mammalian cells is known to result in a biologically active molecule with regard to these additional modifications, as demonstrated herein. Sequences modified by any technique can be tested for biological activity according to known methods including those described in the Examples section in order to determine if that modified sequence is an equivalent, a conservatively modified variant, a biologically function equivalent, a biological variant, or is otherwise similar to a sequence described or listed herein.

5.30 Treating a Tumor

In certain embodiments, a method of treating a tumor in a mammal in need thereof, is described. The exemplary mammal is a human. The preferred method of treating the tumor is by administering an anti-tumor effective amount of a C/EBPβisoform to the mammal. Anti-tumor isoforms of C/EBPβ include C/EBPβ-1 and C/EBPβ-3. However, C/EBPβ-2 is not an anti-tumor agent. It is contemplated that C/EBPβ-2 is a tumor promoter, at least in certain circumstances. Thus, in certain preferred embodiments, the C/EBPβ isoform is substantially free of a C/EBPβ-2 isoform. In certain more preferred embodiments, the C/EBPβ isoform is essentially free of a C/EBPβ-2 isoform. In embodiments wherein the C/EBPβ isoform is administered as a protein, the term “essentially free” includes the meaning that the C/EBPβ-2 protein is not detected in antibody based assays known to one with skill in the art (e.g., enzyme-linked immunosorbent assay, ELISA; Western blotting).

In certain preferred embodiments, an anti-tumor effective amount of a C/EBPβ isoform protein is administered to the mammal. In certain highly preferred embodiments, an anti-tumor effective amount of a C/EBPβ isoform encoding polynucleotide is administered to the mammal wherein an anti-tumor effective amount of a C/EBPβ isoform protein is expressed in the mammal from the polynucleotide. Whether administered as a protein or encoded in a polynucleotide, an anti-tumor C/EBPβ isoform can be administered and it is preferred that the C/EBPβ isoform is a C/EBPβ-1 isoform and/or a C/EBPβ-3 isoform. Alternatively, the C/EBPβ isoform is a conservatively modified variant thereof.

5.40 Polynucleotide Compositions and Methods of Use Thereof

In certain exemplary embodiments, the administration of an anti-tumor C/EBPβ isoform includes introducing a nucleic acid to a cell, wherein the nucleic acid includes a polynucleotide segment encoding the anti-tumor C/EBPβ isoform. It is preferred to administer the nucleic acid directly to a cell in the mammal and that an anti-tumor amount of C/EBPβ is expressed in the cell. These methods may be referred to as in vivo gene therapy with an anti-tumor C/EBPβ isoform. However, other methods are acceptable including ex vivo administration to a cell taken from the mammal or administration to a cell from another organism that is compatible with introduction into the mammal (not immunogenic in the mammal).

It is preferred that the nucleic acid including the polynucleotide segment encoding the anti-tumor C/EBPβ isoform, further include an expression vector operatively linked to the segment and include at least one control element for expression of the C/EBPβ isoform in the cell. In a general sense, the polynucleotide segment is often referred to as an “insert” or an “expression vector insert”. The construction and use of expression vectors in genetic expression systems is well known in the art. In general, an insert encoding a factor to be expressed is cloned into the expression vector utilizing a bank of restriction sites located such that control elements in the vector will regulate expression of the insert (expression of a product from the insert). Genetic control elements may also be included in the insert. Exogenous genetic elements for driving expression include, but are not limited to: promoters, enhancers, ribosomal binding sites, polyadenylation signals, etc. A number of these elements are described in U.S. Pat. No. 5,910,488 to Nabel et al.; incorporated herein by reference. The terms “control elements”, “genetic regulatory elements”, “genetic control elements”, and grammatically similar expressions are used interchangeably herein. In certain embodiments, preferred inserts include, but are not limited to: the C/EBPβ-1 polynucleotide segment and the C/EBPβ-3 polynucleotide segment. In other embodiments, a preferred insert includes a segment encoding C/EBPβ-1 that is mutated to prevent expression of C/EBPβ-2 and/or mutated to enhance expression of C/EBPβ-1, infra.

Numerous gene expression systems or expression vectors, methods for the construction of the expression vectors, methods for the insertion of desired nucleic acid sequences into the vectors, and methods for optimizing gene product expression from the vectors in various cells are known to those with skill in the art and may be used in conjunction with the present invention. The specific insertion of nucleic acid sequences encoding C/EBPβ-1 and C/EBPβ-3 into an expression vector of choice will be obvious to one with skill in the art including the techniques of molecular cloning which are described by numerous sources (see, Sambrook et al., Molecular Cloning: A Laboratory Manual, Second Edition (1989) Cold Spring Harbor Laboratory Press), incorporated herein by reference). In certain preferred embodiments, the expression vector contains the nucleic acid sequence encoding C/EBPβ-1 and/or C/EBPβ-3 along with various genetic elements that promote the constitutive or inducible expression of the desired gene product.

5.41 Expression Vectors

Many desirable expression vectors, including plasmid expression vectors, are available through commercial sources (e.g., Roche, Stratagene, Invitrogen, Promega, etc) and are useful for the expression of C/EBPβ-1 and C/EBPβ-3 in mammalian cells. In certain embodiments, the nucleic acid is transcribed and it is preferred that the resulting transcript is translated into a protein. Thus, in certain embodiments, expression includes both transcription of a gene and translation of a RNA into a gene product, including, but not limited to: C/EBPβ-1 and/or C/EBPβ-3. In other embodiments, expression only includes transcription of the nucleic acid, for example, to generate antisense constructs.

Particularly useful vectors are contemplated to be those vectors in which a coding portion of the DNA segment, whether encoding a full length protein, polypeptide or smaller peptide, is positioned under the transcriptional control of a genetic control element. One highly preferred control element includes a promoter. In certain aspects “promoter” refers to a DNA sequence recognized by the synthetic machinery of the cell, or introduced synthetic machinery, required to initiate the specific transcription of a gene. The phrases “operatively positioned”, “under control” “regulates”, or “under transcriptional control” include the meaning that the promoter is in the correct location and orientation in relation to the nucleic acid to control RNA polymerase initiation and expression of the gene. These terms are known to one of ordinary skill in the art.

The promoter may be in the form of the promoter that is naturally associated with a gene, as may be obtained by isolating the 5′ non-coding sequences located upstream of the coding segment or exon, for example, using recombinant cloning and/or polymerase chain reaction (PCR™) technology (see U.S. Pat. No. 4,683,202 to Mullis; U.S. Pat. No. 4,683,195 to Mullis et al.; U.S. Pat. No. 4,800,159 to Mullis et al.; U.S. Pat. No. 4,965,188 to Mullis et al.; U.S. Pat. No. 5,656,493 to Mullis et al.; each patent incorporated herein by reference).

In other embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with a gene in its natural environment. Such promoters may include promoters normally associated with other genes, and/or promoters isolated from any other bacterial, viral, eukaryotic, or mammalian cell, and/or promoters made by the hand of man that are not “naturally occurring,” i.e., containing elements from different promoters, or mutations that increase, decrease, or alter expression.

Naturally, it will be important to employ a promoter that effectively directs the expression of the DNA segment in the cell type, organism, or mammal, chosen for expression; the exemplary mammal being a human. The use of promoter and cell type combinations for protein expression, including various types of human cells, is known to those of skill in the art of molecular biology (for example, see Sambrook et al. (1989), supra). The promoters employed may be constitutive, or inducible, and can be used under the appropriate conditions to direct high level expression of the introduced DNA segment, such as is advantageous in the large-scale production of recombinant proteins or peptides.

Generally at least one module in a promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region about 30-110 base pairs upstream of the start site, although a number of promoters have been shown to contain functional elements downstream of the start site as well. The spacing between promoter elements is often observed to be flexible, so that promoter function is preserved when elements are inverted or moved relative to one another. In the tk promoter, the spacing between promoter elements can be increased to 50 base pairs apart before activity begins to decline. Depending on the promoter, it appears that individual elements can function either cooperatively or independently to activate transcription.

In certain embodiments, the particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter. In other embodiments, a particular promoter that directs expression to a certain tissue or allows for regulation of expression by an additional control element may be desired. The selection and use of such particular promoters will be apparent to those with skill in the art (see, e.g., U.S. Pat. No. 5,858,774 to Malbon et al.; Gene-Expression Systems (1998) Fernandez et al., eds. Academic Press; M. Kriegler, Gene Transfer and Expression: A Laboratory Manual (1991) Oxford University Press; Gene Therapy of Cancer (1999) Lattime et al., (eds.) Academic Press; and Gene Expression: General and Cell Type Specific (1993) M. Karin (ed.) Birkhauser; each reference being incorporated herein by reference).

In various embodiments, the human cytomegalovirus (CMV) immediate early gene promoter, the SV40 early promoter, and the Rous sarcoma virus long terminal repeat can be used to obtain high-level expression of the nucleic acid. The use of other viral or mammalian cellular promoters which are well-known in the art to achieve expression are contemplated as well, provided that the levels of expression are sufficient for a given purpose as stated in the specification including the claims. For example, in certain embodiments herein, the purpose is the treatment of a tumor, tumor prophylaxis, inhibition of tumor growth, inhibition of proliferation, inhibition of angiogenesis, and induction of cell death. Elements and promoters from the following genes and viral genomes may be useful, in the context of the present invention, to regulate the expression of a gene: β-Actin, metallothionein, H2B (TH2B) histone, mouse or type I collagen, SV40, polyoma virus, retroviral promoters, papilloma virus, hepatitis B virus, human immunodeficiency virus, cytomegalovirus, RSV LTR, whey acidic protein (WAP), and β-casein.

Inducible elements and promoter can be derived from the following genes and viral genomes with the inducing agent in parentheses: MT II (phorbol ester (TFA) and heavy metals), mouse mammary tumor virus (MMTV, stimulated by glucocorticoids), adenovirus 5 E2 (E1a), and SV40 (TPA). In certain embodiments, it is preferable to employ tumor-specific promoters (i.e. promoters that are more active in tumor cells than in non-tumor cells). Preferred examples of such a promoters include, the α-fetoprotein promoter, the amylase promoter (especially, the murine amylase promoter), the cathepsin E promoter, the M1 muscarinic receptor promoter, the γ-glutamyl transferase promoter, and especially the CMV promoter. These lists are not intended to be exhaustive of all the possible useful promoter elements involved in the promotion of expression, but they are exemplary thereof. Additional control elements are discussed, infra.

By employing a promoter with well-known properties, the level and pattern of expression of a polynucleotide following transfection can be optimized. For example, selection of a promoter which is active in specific cells, such as tyrosinase (melanoma), alpha-fetoprotein and albumin (liver tumors), CC10 (lung tumor) and prostate-specific antigen (prostate tumor) will permit tissue-specific expression of C/EBPβ-1 and C/EBPβ-3 polynucleotides. This list is not intended to be exhaustive of all the possible elements useful in the promotion of C/EBPβ-1 and C/EBPβ-3 expression but, merely, to be exemplary thereof. In certain preferred embodiments, the promoter of choice is the CMV promoter which remains active with high levels of C/EBPβ-3 expression. The CMV promoter and methods of use thereof, are described in U.S. Pat. Nos. 5,385,839 and 5,168,062 to Stinski, each patent incorporated herein by reference.

Enhancers were originally detected as genetic elements that increased transcription from a promoter located at a distant position on the same molecule of DNA. They are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter has one or more elements that direct initiation of RNA synthesis at a particular site and in a particular orientation, whereas enhancers generally lack such specifics. Promoters and enhancers are often overlapping and contiguous, often seeming to have a very similar modular organization. Additionally any promoter and enhancer combination could also be used to drive expression. Additional promoters and enhancers are described in the Eukaryotic Promoter Database (Rouaïda Cavin Périer et al. (1999) Nuc Acid Res 27:307-309, incorporated herein by reference and located on the World Wide Web at the URL “http//www.epd.isb-sib.ch”).

In embodiments that use an expression vector, it is preferred that the vector contain an insert of a C/EBPβ-1 and C/EBPβ-3 isoform, conservatively modified variant thereof, or a biologically functional equivalent genetic sequence of C/EBPβ-1 and C/EBPβ-3. The C/EBPβ-1 and C/EBPβ-3 genes should be positioned in the vector relative to control elements such that the C/EBPβ-1 and C/EBPβ-3 genes are transcribed and ultimately translated in a desired environment in the cell targeted by the treatment. Preferred control elements include, but are not limited to: promoters, enhancers, polyadenylation signals, and translation control elements. The control elements may regulate the biological processes of gene expression such that C/EBPβ-1 and C/EBPβ-3 are expressed from the vector when stimulated by an catalyst applied during treatment.

Control may regulate translation in addition to transcription. Thus, another preferred control element includes an internal ribosome entry site (IRES), described in U.S. Pat. No. 4,937,190 to Palmenberg et. al., incorporated herein by reference. The IRES facilitates the delivery of two proteins into animal cells using a single-transcript vector (STV). In such constructs a multiple cloning site (MCS) is located immediately downstream of a single promoter and is followed by the IRES sequence and a second multiple cloning site (or at least one unique restriction site for inserting a second gene sequence). This configuration enables two gene sequences on a single transcript to both be translated into protein. This system has been used with retroviral IRES-STVs in which a selectable drug marker gene was inserted immediately following the IRES. After drug selection, up to 99% of infected cells expressed the MCS-inserted gene as well. The IPES technology is available through Clontech Laboratories, Inc., Palo Alto, Calif. Retroviral vectors are being prepared by the inventor which include a C/EBPβ-1 insert in a first MCS upstream of an IRES, which is upstream of a C/EBPβ-3 segment inserted into a second MCS. These vectors are further being prepared with a mutation in the 2^{nd} in-frame ATG of the C/EBPβ-1 isoform to prevent C/EBPβ-2 isoform expression and the creation of a Kozak sequence at the ATG for the C/EBPβ-1 isoform to enhance translation of C/EBPβ-1. A Kozak sequence is a translation initiation enhancer sequence. (For examples of translational enhancer compositions and methods of use thereof, see U.S. Pat. No. 5,807,707 to Andrews et al., incorporated herein by reference; U.S. Pat. No. 5,723,332 to Chernajovsky, incorporated herein by reference; U.S. Pat. No. 5,891,665 to Wilson, incorporated herein by reference).

In certain embodiments of the invention, the delivery of an expression vector in a cell may be identified in vitro or in vivo by including a marker in the expression vector. The marker would result in an identifiable change to the transfected cell permitting easy identification of expression. Usually the inclusion of a drug selection marker aids in cloning and in the selection of transformants. Alternatively, enzymes such as herpes simplex virus thymidine kinase (tk) (eukaryotic) or chloramphenicol acetyltransferase (CAT) (prokaryotic) may be employed. Immunologic markers also can be employed. Red and Green Fluorescent Protein Markers are available from Clontech, supra. The selectable marker employed is not believed to be important, so long as it is capable of being expressed along with the polynucleotide encoding C/EBPβ-1 and C/EBPβ-3. Further examples of selectable markers and methods for constructing and using markers are well known to one of skill in the art.

One typically will include a polyadenylation signal (polyA) to effect proper polyadenylation of the transcript. The nature of the polyadenylation signal is not believed to be crucial to the successful practice of the invention, and any such sequence may be employed. The inventor prefers the bovine growth hormone (bGH) polyA for or plasmid vectors, the Moloney murine leukemia virus (MoMLV) polyA for retroviral vectors, the SV40 polyA for adenoviral vectors, in that it was convenient and known to function well in the target cells employed. Also contemplated as an element of the expression construct, but not preferred, is a terminator separate from the polyA. These elements can serve to enhance message levels and to minimize read through from the construct into other sequences.

Other expression vectors known in the art that may be useful for the expression of encoded genes in mammalian cells include, but are not limited to: pUC and Bluescript™ plasmid series, direct uptake of naked DNA, as well as receptor-mediated uptake of DNA complexes, DNA-liposome complexes (described in U.S. Pat. No. 5,676,954 to Brigham, incorporated herein by reference), cosmids, and phage constructs. A general resource for the construction and use of plasmid, recombinant, and viral vectors for gene-therapy that can be used, in certain embodiments, in light of the present invention is U.S. Pat. No. 5,545,563 to Darlington et al., incorporated herein by reference.

Once the expression construct has been delivered into the cell the nucleic acid encoding the gene of interest may be positioned and expressed at different sites within the cell. In certain embodiments, the nucleic acid encoding the gene may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In certain embodiments, gene transfer may more easily be performed under ex vivo conditions. Ex vivo gene therapy commonly refers to the isolation of cells from the mammal, the delivery of a nucleic acid into the cells, in vitro, and then the return of the modified cells back into the mammal. This may involve the surgical removal of tissue/organs from the mammal or the primary culture of cells and tissues. U.S. Pat. No. 5,399,346 to Anderson et al., incorporated herein by reference, disclose several ex vivo therapeutic methods. The preferred animal herein is a mammal and an especially preferred animal is a human. Particularly good methods of administration of plasmid expression vectors is by injection of naked DNA, inhalation of aerosolized naked DNA, incorporation into liposomes and uptake by treated cells, association with cationic liposomes by charge-charge interactions, instillation, and injection or aerosolization in general.

Advantages to plasmid expression vector based expression systems include: plasmids generally do not integrate into the genomic DNA of the host cell, plasmid systems are typically less immunogenic than viral based expression systems, broad range of host expression cell available, selective expression in certain host cells possible, temporal expression possible, and the bystander effect allows effective treatment even when gene transmission rates are low. The bystander effect is an known in the art as a substantial therapeutic effect resulting from transduction of a relatively small population of targeted cells. For example, an illustration of the bystander effect would be the regression of a tumor following gene therapy of the tumor in which fewer than 10% of the tumor cells were transformed by the gene therapy. The bystander effect is described in Gene Therapy of Cancer (1999) Lattime et al., (eds.) Academic Press, especially Chapter 10, incorporated herein by reference. The bystander effect is described also in U.S. Pat. No. 5,866,340 to Vogelstein et al., incorporated herein by reference. The description of the benefit plasmid based gene therapy by the bystander effect is not meant to limit the present invention, but merely to be illustrative thereof. The bystander effect is expected to benefit viral expression vector based therapy and other embodiments as well.

In certain embodiments of the present invention, expression vectors (including viral-based expression vectors, infra) are used to transduce various cell types including, but not limited to: HMECs (normal control), MCF10A the panel of mammary carcinoma cell lines (MDA468, MDA 231, MCF7-neo, MCF7-218, MCF7, T47D, and BT474), and cells derived from primary breast tumors. In certain embodiments, expression vectors (including viral-based expression vectors) are used for ex vivo or in vivo transduction of mammalian tissues or cell types including, but not limited to: epithelial, endothelial, hepatocyte, lymphoid, myeloid, vascular endothelium, and mammary epithelium. In certain embodiments, the influence of the expression of C/EBPβ-1 and C/EBPβ-3 is independently determined on the growth potential of the cells and tissues.

5.42 Viral-Based Expression Vectors

In certain embodiments, viral expression vectors are preferred. Preferred viral based expression vectors include hybrid retrovirus/Epstein Barr virus vector (e.g., pLZRSpBMN-Z described in U.S. Pat. No. 5,830,725 to Nolan et al., incorporated herein by reference; see Examples 2 and 3, FIGS. 9A-D, and 10A-C) and adenoviral expression vectors (e.g., pGEM-RecA; see Examples 4 and 5).

The viral vectors described in certain embodiments herein are non-replicating, meaning that no further virus spread occurs after infection. To distinguish this process from a natural virus infection where the virus continues to replicate and spread, the terms “transduce”, transduced”, and “transduction” are commonly used and may be used herein.

Numerous viral-based viral expression systems are described in the prior art and the use of any of these systems, or any system developed in the future, in conjunction with the compositions and methods of the present invention and in light of the present disclosure, is contemplated. However, it is preferred that the viral expression system is compatible with administration to a mammal and that it facilitate the expression of C/EBPβ-1, C/EBPβ-3, and/or other compositions of the present invention in a mammalian cell.

5.43 Retrovirus

The retroviruses are a group of single-stranded RNA viruses characterized by an ability to convert their RNA to double-stranded DNA to infected cells by a process of reverse-transcription (Retroviruses (1997) Coffin et al. (eds.), Cold Spring Harbor Laboratory; incorporated herein by reference; Gene Therapy of Cancer (1999) Lattime et al., (eds.) Academic Press, Chapter 4, incorporated herein by reference). Typically, the resulting DNA then stably integrates into cellular chromosomes as a provirus and directs synthesis of viral proteins. The integration results in the retention of the viral gene sequences in the recipient cell and its descendants. The retroviral genome contains gag, pol, and env genes that code for capsid proteins, polymerase enzyme, and envelope components, respectively. A sequence found upstream from the gag gene, termed ψ contains a signal for the packaging of the viral genome into virions. Two long terminal repeat (LTR) sequences are present at the 5′ and 3′ ends of the viral genome. These contain strong promoter and enhancer elements and also direct integration of the viral nucleic acid into the host cell genome.

In order to construct a retroviral vector, in general, a nucleic acid encoding a promoter is inserted into the viral genome replacing the gag, pol, and env genes producing a replication deficient virus genome. In order to produce virions, a packaging cell line containing the gag, pol, and env genes; but without the LTR and Ψ components is constructed. Numerous packaging cell lines are known to one with skill in the art, are available commercially, and are available through the American Type Culture Collection (ATCC, Rockville, Md.).

When a recombinant plasmid containing a human cDNA, together with the retroviral LTR and ψ sequences is introduced into the packaging cell line (by calcium phosphate precipitation for example), the ψ sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression is enhanced by the division of host cells.

Several concerns regarding the use of retroviral vectors include the potential for disruption of native genes of the host cell through random integration and the possibility of regeneration of a replication-competent particle through recombination in the packaging cell line. However, packaging cell lines are now available that should greatly decrease the likelihood of recombination (Markowitz et al. (1988) Virology 167(2):400-406; Markowitz et al. (1988) J. Virol. (62)1120-1124; Hersdorffer et al. (1990) DNA Cell Biol., (9) 713-723; each incorporated herein by reference). Another limitation to the use of retrovirus vectors in vivo is the limited ability to produce retroviral vector titers greater than 10⁶ U/milliliter. Titers 10- to 1,000-fold higher are preferred for many in vivo applications.

Nevertheless, several innovations in the application of retroviruses demonstrate the utility of retroviral vectors for delivering the anti-tumor agents C/EBPβ-1 and C/EBPβ-3 in conjunction with the present invention. U.S. Pat. No. 5,911,983 to Barranger et al., incorporated herein by reference, describes the use of a retroviral vector for gene therapy of Gaucher disease. U.S. Pat. No. 5,910,434 to Rigg et al., incorporated herein by reference, describes packaging cell lines and methods of generating high titer retrovirus useful for gene therapy. U.S. Pat. No. 5,741,486 to Pathak et al., incorporated herein by reference, describes a method of inhibiting or preventing the formation of replication competent retrovirus particles by providing a retroviral vector that deletes an essential encapsidation sequence upon reverse transcription in the target cells. U.S. Pat. No. 6,017,761 to Rigg et al., describes a method for obtaining retroviral packaging cell lines producing high transducing efficiency retroviral supernatant. The treatment of tumors in a mammal using retroviral vectors and a p53 gene is described in U.S. Pat. No. 5,532,220 to Lee et al., incorporated herein by reference.

5.44 Hybrid Retrovirus

The preferred expression system for high efficiency gene transfer in the present invention includes a hybrid Epstein-Barr virus(EBV)/retroviral vector construct (LZRSpBMN-Z ) (U.S. Pat. No. 5,830,725 to Nolan et al., incorporated herein by reference). In addition to a murine retroviral backbone with a polylinker region to facilitate insertion of cDNAs, the LZRSpBMN-Z vector contains the Epstein-Barr virus Nuclear Antigen (EBNA) gene, EBV origin of replication and nuclear retention sequences (oriP), and a puromycin resistance gene (FIG. 9D). The nuclear replication and retention functions of this vector allow for rapid establishment of recombinant retroviral producer DNA as stable episomes within human retroviral packaging cell lines. Episomes are maintained at 5-20 copies per cell (approximately) for up to 2-3 months, given selection for puromycin resistance, resulting in high viral titers. The retroviral backbone in this vector consists of full-length Moloney murine leukemia virus longer repeat (LTR) and extended ψ packaging sequences derived from the MFG series of retroviral vectors developed by Mulligan and colleagues (U.S. Pat. No. 4,868,116 to Morgan et al., incorporated herein by reference).

Helper-free retrovirus is produced by transfecting the LZRS-based his-C/EBPβ-1, his-C/EBPβ-3, or a control β-gal construct (see Examples 2 and 3) into a 293T-based amphotropic packaging cell line termed (φ)nx-ampho (provided by Gary Nolan, Stanford University, Calif., USA). The 293T-derived cell lines are transfected with high efficiency (50% to 80%, or more of total cells being transfected) using calcium phosphate mediated transfection. The (φ)nx-ampho packaging cell line was specifically developed by G. Nolan to produce high titer, helper free recombinant retrovirus. The improvements were designed to alleviate instability of retroviral production capacity and potential recombination problems. Thus, hygromycin and diphtheria toxin resistance genes were introduced as co-selectable markers for the gag-pol and amphotropic envelope constructs respectively. To reduce the potential for recombination, the gag-pol and envelope constructs are driven by different, non-MoMuLV promoters. The risk of rearrangements is further reduced when LZRS-based constructs are maintained episomally in fnx-based packaging lines, resulting in the safe production of helper-free retroviral stocks.

5.45 Adenovirus

Another method for in vivo delivery, including gene therapy, involves the use of an adenovirus vector. The use of adenoviral vectors for the delivery of gene therapy is known in the art. Adenoviral vectors and methods for use thereof, that include non-native coat proteins for reduced immunogenicity and increased cellular uptake are described in U.S. Pat. No. 5,965,541 to Wickham et al.; the life cycle of adenovirus, adenoviral vector compositions, and methods of use thereof for gene therapy are described in U.S. Pat. No. 5,731,190 to Wickham et al.; the use of adenoviral vectors incorporating a novel tumor suppressor gene in the treatment of cancer is described in U.S. Pat. No. 5,922,688 to Hung et al.; each patent is incorporated herein by reference.

Viral vectors based on the adenovirus are particularly suited for gene transfer and gene therapy because of its mid-sized genome, ease of manipulation, high titer viral particle production, wide target-cell range, and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP, located at 16.8 mμ is particularly efficient during the late phase of infection, and all the mRNA's issued from this promoter possess a 5′-tripartite leader (TL) sequence which makes them preferred mRNA's for translation.

In some cases, recombinant adenovirus is generated from homologous recombination between a shuttle vector and a provirus vector. Due to the possible recombination between two proviral vectors, wild-type adenovirus may be generated from this process. Therefore, it is critical to isolate a single clone of virus from an individual plaque and examine its genomic structure. Use of the YAC system is an alternative approach for the production of recombinant adenovirus.

In certain embodiments, a method of introducing C/EBPβ-1 and C/EBPβ-3 to a mammal is to introduce a replication-deficient adenovirus containing a polynucleotide segment or insert containing the C/EBPβ-1 and C/EBPβ-3 genes. Certain preferred constructs are made replication deficient by deletion of the viral E1B and E3 genes. This avoids viral reproduction inside the cell and transfer to other cells and infection of other people. In other words, the viral infection activity is shut down after it transduces the target cell, but the p20 gene is still expressed inside the cells. Also, adenovirus is able to transfer the p20 gene efficiently into both proliferating and non-proliferating cells. Further, the extrachromosomal location of adenovirus in the infected cells decreases the chance of cellular oncogene activation within the treated mammal (adenoviruses do not generally integrate into the host cell genome). However, the inventor still prefers the hybrid/EBV retroviral vector described above because transduction of non-proliferating cells may be unnecessary in treating proliferating cells in a tumor because transduction of proliferating cells may be adequate.

The nature of the adenovirus vector is not believed to be crucial to the successful practice of the invention. Of course, as discussed above, it is advantageous if the adenovirus vector is replication defective, or at least conditionally defective, The adenovirus may be of any of the 42 different known serotypes or subgroups A-F. Adenovirus type 5 of subgroup C is the preferred starting material in order to obtain the conditional replication-defective adenovirus vector for use in certain embodiments of the present invention. This is because Adenovirus type 5 is a human adenovirus about which a great deal of biochemical and genetic information is known, and it has historically been used for most constructions employing adenovirus as a vector. The preferred adenoviral vector is pGEM-RecA (Examples 4 and 5).

Adenovirus is easy to grow and manipulate and exhibits broad host range in vitro and in vivo. This group of viruses can be obtained in high titers, e.g., 10⁹ 10¹¹ plaque-forming units per ml, and the particles are highly infective. The life cycle of adenovirus does not require integration in to the host cell genome. The foreign genes delivered by adenovirus vectors are episomal and, therefore, have low genotoxicity to host cells. No side effects have been reported in certain studies of vaccination with wild-type adenovirus, demonstrating their safety and therapeutic potential as in vivo gene transfer vectors. However, other findings have shown a limitation with regard to immunogenic responses to adenoviral antigens.

U.S. Pat. No. 5,923,210 to Gregory et al., incorporated herein by reference, and U.S. Pat. No. 5,824,544 to Armentano et al., incorporated herein by reference, describe modifications to adenoviral vectors, and medical uses thereof, that decrease the potential for spontaneous generation of a replication competent adenovirus; thus, making the vectors even more safe for clinic use. The patents involve the deletion of the E1A (both patents) and E1B adenovirus genes (5,923,210) and deletion (5,923,210) or relocation (5,824,544) of the adenovirus IX gene.

U.S. Pat. No. 5,792,453 to Hammond et al., incorporated herein by reference, describes adenoviral vectors useful for gene therapy for peripheral vascular disease and heart disease, including myocardial ischemia. The adenoviral vector is administered by intra-femoral artery or intracoronary injection conducted deeply in the lumen of the one or both femoral or coronary arteries (or graft vessels) in an amount sufficient for transfecting cells in a desired region.

Enhanced gene transfer to cancers arising from epithelial cells using adenoviral vectors and a transfer enhancing reagent, namely ethanol, is described in U.S. Pat. No. 5,789,244 to Heidrun et al., incorporated herein by reference.

Enhanced gene transfer to vascular cells using adenoviral and retroviral vectors and a transfer enhancing reagent, namely polyols, is described in U.S. Pat. No. 5,552,309 to March, incorporated herein by reference.

Successful delivery and expression of the cystic fibrosis transmembrane conductance regulator (CFTR) gene into the tracheobronchial passages of rhesus monkeys including the alveolar sacs using an adenovirus 5 based vector with a CFTR gene insertion and a technique for generating a viral aerosol is described in U.S. Pat. No. 5,952,220 to Sene et al., incorporated herein by reference.

5.46 Treatment with an NLS-PNA-C/EBPβ Expression Vector

Introduction, (for example, by liposomal based “transfection”) of a composition comprising a nuclear localization sequence linked to a peptide nucleic acid which is hybridized to a C/EBPβ expression vector is useful treatment related to certain embodiments herein, including, but not limited to the treatment of a tumor, inhibition of tumorigenesis, inhibition of cell growth, inhibition of cellular proliferation, promotion of cell death, and/or activation of cell death (see, e.g., International application WO 99/13719 to Felgner et al., published Mar. 25, 1999 and Branden et al. (1999) Nature Biotechnology 17:784-787). Preferably, the expressing vector produces C/EBPβ-1 and/or C/EBPβ-3, but not C/EBPβ-2 in the mammalian cell into which it is introduced. One advantage to such a system is that a C/EBPβ isoform gene can be administered without the use of a viral based vector for infection, although any viral based system can also be transported into the cell using NLS-PNA.

In general, the NLS-PNA-C/EBPβ expression vector is administered by transfection of the composition through the cell membrane. The NLS then causes the translocation of the composition to the nucleus where the C/EBPβ isoform is expressed. Preferred embodiments of a C/EBPβ-1 expression vector are modified to prevent expression of C/EBPβ expression vector. Such administration can be by any route known including those specifically identified herein. The preferred method for transfection comprises mixing the composition with a liposome or transfection enhancing lipid and contacting the formulation to a cell, tissue, or organ of the mammal in need of treatment.

5.47 Other Expression Systems

It is believed that the choice of expression vector, including viral-based expression vectors, is limited only by the pharmaceutical administration of the vector to the cell or mammal depending on the embodiment. Thus, it is preferred that the vector does not elicit an adverse immunological (meaning toxic) response in the mammal when treatment of a mammal is concerned. It is preferred that the vector does not support integration into the host cell genome because this may disrupt host cell gene expression. It is preferred, also, that the vector system support a level of expression of a composition of the present invention in a chosen cell that is therapeutically effective according to the particular embodiment (e.g., treating a tumor, inhibiting cellular proliferation, or stimulating cellular differentiation). Therefore, in addition to the non-infectious vectors, retroviral vectors, hybrid EBV/retroviral vectors, and adenoviral vectors; other expression vector systems, both known and to be developed, are contemplated to be useful in certain embodiments in light of the present disclosure.

Alternative expression systems are pointed out below by way of example only. In certain embodiments of the present invention, the expression construct can be introduced into the cell using adenovirus assisted transfection. Increased transfection efficiencies have been reported in cell systems using adenovirus mediated systems as described in U.S. Pat. No. 5,928,944 to Seth et al. and U.S. Pat. No. 5,830,730 to German et al., each patent incorporated herein by reference. Other viral vectors may be employed as expression constructs in the present invention including, but not limited to: avipox, suipox, iridoviruses, picornavirus, calicivirus, and togavirus (all described in U.S. Pat. No. 5,656,465 to Panicali et al., incorporated herein by reference); a vaccinia virus modified for use in gene therapy (U.S. Pat. No. 5,858,373 to Paoletti et al., incorporated herein by reference); and gene therapy of liver tumors utilizing transcriptional elements of alpha-fetoprotein incorporated into SIN vectors is described in U.S. Pat. No. 5,843,776 to Tamaoki et al., incorporated herein by reference.

In vitro and in vivo gene therapy including the eye using LUX viral vector and Rb insert is described in U.S. Pat. No. 5,858,771 to Lee et al., incorporated herein by reference. Ocular gene therapy using recombinant vector and adenovirus vector is described in U.S. Pat. No. 5,827,702 to Cuthbertson, incorporated herein by reference. Gene therapy of the myocardium utilizing intra-femoral artery or intracoronary injection of adenoviral gene therapy vectors deep in the lumen of one or both femoral or coronary arteries (or graft vessels) is described in U.S. Patent Hammond et al., incorporated herein by reference.

U.S. Pat. No. 5,770,580 to Ledley et al., incorporated herein by reference, describes somatic gene therapy to cells associated with fluid spaces, such as follicles of the thyroid, the synovium of the joint, the vitreous of the eye and the inner or middle ear. Formulated DNA expression vectors are introduced with or without formulation elements into fluid spaces under conditions in which cells associated with the fluid space can incorporate the formulated DNA expression vector. Formulated DNA expression-mediated gene therapy allows treatment of diseases involving cells associated with fluid spaces. Recombinant viral and plasmid vectors for gene-therapy directed to the lung are described in U.S. Pat. No. 5,240,846 to Collins et al., incorporated herein by reference.

Generation of high titers of recombinant adeno-associated virus (AAV) vectors and the application of AAV vectors in gene therapy is described in U.S. Pat. No. 5,658,776 to Flotte et al., incorporated herein by reference. AAV-mediated gene therapy is also described in Gene Therapy of Cancer (1999) Lattime et al., (eds.) Academic Press, Chapter 6, incorporated herein by reference.

5.50 Production and Purification of C/EBPβ-1 and C/EBPβ-3 Proteins

In certain embodiments, C/EBPβ-1 and C/EBPβ-3 polypeptides are used for treatments described in the present invention. Although the C/EBPβ-1 and C/EBPβ-3 polypeptides can be isolated from natural sources such as rat, mouse, or human cells; it is preferred that they be produced using recombinant techniques due to the increased risk of contamination by pathogens when derived from native sources. The cloning of and propagation of the human C/EBPβ nucleotide sequence in plasmid vectors are described in U.S. Pat. No. 5,215,892 to Kishimoto et al. and U.S. Pat. No. 5,360,894 to Kishimoto et al., each patent incorporated herein by reference. Additional methods for cloning and propagation of nucleotide sequences in general, and the C/EBPβ-1 and C/EBPβ-3 nucleotide sequences in particular are known to one with ordinary skill in the art. Methods for obtaining such sequences from different sources (i.e., murine, rat, chicken, xenopus, etc.) are also known.

In general, C/EBPβ-1 and C/EBPβ-3 proteins can be made by inserting their respective nucleic acid sequences into an expression vector suitable for expression in the host cell of choice including bacteria (e.g., BL21-pLysS, which does not phosphorylate the protein product), yeast (e.g., SF9), insect (phosphorylated peptides, used in conjunction with a baculovirus vector), and mammalian host cells which provide wild type phosphorylation. These recombinant expression systems are known in the art and commercially available.

The present invention also provides purified, and in certain preferred embodiments, substantially purified C/EBPβ-1 and C/EBPβ-3 polypeptides. The term “purified C/EBPβ-1 and C/EBPβ-3 polypeptides” as used herein, is intended to refer to a C/EBPβ-1 or a C/EBPβ-3 proteinaceous composition, isolatable from endogenous or recombinant sources, wherein the C/EBPβ-1 or C/EBPβ-3 polypeptide is purified to any degree relative to its naturally-obtainable state, i.e., relative to its purity within a cellular extract. A purified C/EBPβ-1 and C/EBPβ-3 polypeptide therefore also refers to a wild-type or mutant C/EBPβ-1 and C/EBPβ-3 polypeptide free from the environment in which it naturally occurs. In certain embodiments purified C/EBPβ-1 and purified C/EBPβ-3 can be combined together or mixed for use in treatment.

Generally, “purified” will refer to a C/EBPβ-1 or C/EBPβ-3 polypeptide composition that has been subjected to fractionation to remove various non-C/EBPβ-1 or C/EBPβ-3 proteins, polypeptides, or peptides, and which composition substantially retains its C/EBPβ-1 or C/EBPβ-3 activity, as may be assessed, for example, by a C/EBPβ-1 or C/EBPβ-3 assay, such as inhibiting cell proliferation or promoting or inducing cell death. Several of these assays are described in the Examples section, infra.

Where the term “substantially purified” is used, this will refer to a composition in which the C/EBPβ-1 or C/EBPβ-3 polypeptide forms the major component of the composition, such as constituting about 51% of the proteins in the composition or more. In preferred embodiments, a substantially purified protein will constitute more than 70%, 80%, 90%, or even more than 95% of the proteins in the composition. In even more preferred embodiments, a substantially purified protein will constitute 99%, or even more than 99% of the proteins in the composition. The amount of a particular protein can be determined by comparing the opacity of the protein band with other proteins in the same lane after running the sample by SDS/PAGE and visualizing the proteins by silver staining as is known in the art.

A peptide, polypeptide or protein that is “purified to homogeneity,” as applied to the present invention, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Various methods for quantifying the degree of purification of C/EBPβ-1 or C/EBPβ-3 polypeptides will be known to those of skill in the art in light of the present disclosure. These include, for example, determining the specific C/EBPβ-1 or C/EBPβ-3 protein activity of a fraction, or assessing the number of polypeptides within a fraction by gel electrophoresis. Assessing the number of polypeptides within a fraction by SDS/PAGE analysis will often be preferred in the context of the present invention as this is straightforward.

To purify a C/EBPβ-1 or C/EBPβ-3 polypeptide a natural or recombinant composition comprising at least some C/EBPβ-1 or C/EBPβ-3 polypeptides will be subjected to fractionation to remove various non-C/EBPβ-1 or C/EBPβ-3 components from the composition. In addition to those techniques, various other techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques.

Another example is the purification of an C/EBPβ-1 or C/EBPβ-3 fusion protein using a specific binding partner. Such purification methods are routine in the art. As the Kishimoto 5,215,892 patent provides a C/EBPβ nucleotide sequence and the present invention provides sequences capable of expressing C/EBPβ-1 and C/EBPβ-3, but not C/EBPβ-2; then any fusion protein purification method can now be practiced. This is exemplified by the generation of an C/EBPβ-1 or C/EBPβ-3-glutathione S-transferase fusion protein, expression in E. coli, and isolation (including to homogeneity) using affinity chromatography on glutathione-agarose or the generation of a polyhistidine tag on the N- or C-terminus of the protein, and subsequent purification using Ni-affinity chromatography. Given the DNA and proteins described in the present invention, any purification method can now be employed.

The preferred method of protein isolation is by affinity chromatography of a 6×His Tag included in the nucleic acid encoding the C/EBPβ-1 and C/EBPβ-3 protein products (see the Examples section). The 6×His Tag adds an additional 0.84 kDa to the overall molecular weight of the protein product and does not interfere with the C/EBPβ-1 and C/EBPβ-3 activity. The expression product is then purified by chromatography on a nickel-nitrilotriacetic acid (Ni-NTA) column. If the 6×His Tag is found to interfere with an activity of the protein product for a specific purpose, the tag can be removed. All of these protein product purification techniques are known to one with skill in the art (see e.g., Petty (1996) Current Protocols in Molecular Biology Vol. 2, John Wiley and Sons Publishers, incorporated herein by reference) and are commercially available from Qiagen (Valencia, Calif. 91355).

Although preferred for use in certain embodiments, there is no general requirement that the C/EBPβ-1 and C/EBPβ-3 polypeptide always be provided in their most purified state. Indeed, it is contemplated that less substantially purified C/EBPβ-1 or C/EBPβ-3 polypeptide which are nonetheless enriched in C/EBPβ-1 or C/EBPβ-3 protein compositions, relative to the natural state, will have utility in certain embodiments. These include, for example, antibody generation where subsequent screening assays using purified C/EBPβ-1 or C/EBPβ-3 proteins are conducted. Methods exhibiting a lower degree of relative purification may have advantages in total recovery of protein product, or in maintaining the activity of an expressed protein. Inactive products also have utility in certain embodiments, such as, e.g., in antibody generation.

Turning to the expression of the proteins, once a suitable clone or clones have been obtained, whether they be cDNA based or genomic, one may proceed to prepare an expression system. The engineering of DNA segment(s) for expression in a prokaryotic or eukaryotic system may be performed by techniques generally known to those of skill in recombinant expression. It is believed that virtually any expression system may be employed in the expression of the proteins.

Both cDNA and genomic sequences are suitable for eukaryotic expression, as the host cell will generally process the genomic transcripts to yield functional mRNA for translation into protein. Generally speaking, it may be more convenient to employ as the recombinant gene a cDNA version of the gene. In general, it is believed that the use of a cDNA version will provide advantages in that the size of the gene will generally be much smaller and more readily employed to transfect the targeted cell than will a genomic gene, which will typically be up to an order of magnitude or more larger than the cDNA gene. Although, it is contemplated that a genomic version of a particular gene may be employed where desired. In the present case, however, C/EBPβ (including C/EBPβ-1 and C/EBPβ-3) does not contain an intron. Thus, the genomic and cDNA sequences are similar without intervening intron(s).

In expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette, in certain embodiments, is a terminator. These elements can serve to enhance message levels and to minimize read through from the cassette into other sequences.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements. As described in certain embodiments herein, the second in-frame ATG (for human, mouse, and rat nucleotide sequences) is altered to prevent expression of the C/EBPβ-2 isoform.

It is proposed that proteins, polypeptides or peptides may be co-expressed with other selected proteins, wherein the proteins may be co-expressed in the same cell or a gene(s) may be provided to a cell that already has another selected protein. Co-expression may be achieved by co-transfecting the cell with two distinct recombinant vectors, each bearing a copy of either of the respective DNA. Alternatively, a single recombinant vector may be constructed to include the coding regions for both of the proteins, which could then be expressed in cells transfected with the single vector. In either event, the term “co-expression” herein refers to the expression of both the gene(s) and the other selected protein in the same recombinant cell. In certain embodiments, a C/EBPβ-1 sequence and a C/EBPβ-3 sequence are co-expressed. It is preferred that the C/EBPβ-1 sequence is altered to prevent expression of the C/EBPβ-2 isoform of C/EBPβ.

As used herein, the terms “engineered” and “recombinant” cells or host cells are intended to refer to a cell into which an exogenous DNA segment or gene, such as a cDNA or gene encoding an protein has been introduced. Engineered cells are thus cells having a nucleic acid, a gene, or genes introduced through the hand of man. Therefore, engineered cells are distinguishable from naturally occurring cells which do not contain a recombinantly introduced exogenous DNA segment or gene. Recombinant cells include those having an introduced cDNA or genomic gene, and may also include genes positioned adjacent to a promoter not naturally associated with the particular introduced gene.

To express a recombinant protein, polypeptide or peptide, whether mutant or wild-type, in accordance with the present invention one would prepare an expression vector that comprises a wild-type, or mutant protein-encoding nucleic acid under the control of one or more promoters. To bring a coding sequence “under the control of” a promoter, one positions the 5′ end of the transcription initiation site of the transcriptional reading frame generally between about 1 and about 50 nucleotides “downstream” (i.e., 3′) of the chosen promoter. The “upstream” (i.e., 5′) promoter stimulates transcription of the DNA and promotes expression of the encoded recombinant protein. This is a meaning of “recombinant expression” in this context.

Many standard techniques are available to construct expression vectors containing the appropriate nucleic acids and transcriptional/translational control sequences in order to achieve protein, polypeptide or peptide expression in a variety of host-expression systems. Cell types available for expression include, but are not limited to, bacteria, such as E. coli and B. subtilis transformed with recombinant bacteriophage DNA, plasmid DNA, or cosmid DNA expression vectors. The use of cosmid DNA and artificial chromosomes is common particularly in yeast or mammalian expression systems.

Certain examples of prokaryotic hosts are E. coli strains:DH5α (preferred), HB101, E. coli BL21, E. coli BL21-pLysS, E. coli BL21-pLysE, RR1, E. coli LE392, E. coli B, E. coli X 1776 (ATCC No. 31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is often transformed using derivatives of pBR322, a plasmid derived from an E. coli species. pBR322 contains genes for ampicillin and tetracycline resistance and thus provides easy method for identifying transformed cells. The pBR plasmid, or other microbial plasmid or phage must also contain, or be modified to contain, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as E. coli LE392.

Further useful vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors. In certain preferred embodiments, the T7 promoter is used.

The following details concerning recombinant protein production in bacterial cells, such as E. coli, are provided by way of exemplary information on recombinant protein production in general, the adaptation of which to a particular recombinant expression system will be known to those of skill in the art.

Bacterial cells, for example, E. coli, containing the expression vector are grown in any of a number of suitable media, for example, LB. The expression of the recombinant protein may be induced, e.g., by adding IPTG to the media or by switching incubation to a higher temperature. After culturing the bacteria for a further period, generally of between 2 and 24 hours, the cells are collected by centrifugation and washed to remove residual media.

The bacterial cells are then lysed, for example, by disruption in a cell homogenizer or by sonication (preferred) and centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed.

If the recombinant protein is expressed in the inclusion bodies, as is the case for C/EBPβ isoforms, the inclusion bodies can be washed in any of several solutions to remove some of the contaminating host proteins, then solubilized in solutions containing high concentrations of urea (e.g. 8M) or chaotropic agents such as guanidine hydrochloride in the presence of reducing agents, such as β-mercaptoethanol or DTT (dithiothreitol). These techniques are known in the art.

In certain embodiments, it is preferred to incubate the protein for several hours under conditions suitable for the protein to undergo a refolding process into a conformation which more closely resembles that of the native protein. Such conditions generally include low protein concentrations, less than 500 mg/ml, a reducing agent (high levels of a reducing agent are preferred for the present invention), concentrations of urea less than 2 M and often the presence of reagents such as a mixture of reduced and oxidized glutathione which facilitate the interchange of disulfide bonds within the protein molecule.

The refolding process can be monitored, for example, by SDS-PAGE, or with antibodies specific for the native molecule (which can be obtained from animals vaccinated with the native molecule or smaller quantities of recombinant protein). Following refolding, the protein can then be purified further and separated from the refolding mixture by chromatography on any of several supports including ion exchange resins, gel permeation resins or on a variety of affinity columns. As described supra, a polyhistidine tag and Ni-agarose chromatography are preferred.

For expression in Saccharomyces, the plasmid YRp7, for example, is commonly used. This plasmid already contains the trpl gene which provides a selection marker for a mutant strain of yeast lacking the ability to grow in tryptophan, for example ATCC No. 44076 or PEP4-1. The presence of the trpl lesion as a characteristic of the yeast host cell genome then provides an effective environment for detecting transformation by growth in the absence of tryptophan.

Suitable promoting sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are may also by ligated into the expression vector 3′ of the sequence desired to be expressed to provide polyadenylation of the mRNA and termination.

Other suitable promoters, which have the additional advantage of transcription controlled by growth conditions, include the promoter region for alcohol dehydrogenase, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to micro-organisms, cultures of cells derived from multicellular organisms may also be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus (CaMV) or tobacco mosaic virus, (TMV)) or transformed with recombinant plasmid expression vectors (e.g., Ti plasmid) containing one or more protein, polypeptide or peptide coding sequences.

In a useful insect system, Autograph californica nuclear polyhedrosis virus (AcNPV) is used as a vector to express foreign genes. The virus grows in Spodoptera frugiperda cells. The protein, polypeptide or peptide coding sequences are cloned into non-essential regions (for example the polyhedrin gene) of the virus and placed under control of an AcNPV promoter (for example the polyhedrin promoter). Successful insertion of the coding sequences results in the inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., U.S. Pat. No. 4,215,051, Smith, incorporated herein by reference).

Examples of useful mammalian host cell lines are VERO and HeLa cells, Chinese hamster ovary (CHO) cell lines, W138, BHK, COS-7, 293, HepG2, 3T3, RIN and MDCK cell lines. In addition, a host cell strain may be chosen that modulates the expression of the inserted sequences, or modifies and processes the gene product in the specific fashion desired. Such modifications (e.g., glycosylation, phosphorylation) and processing (e.g., cleavage) of protein products may be important for the function of the protein in certain embodiments. Different host cells have characteristic and specific mechanisms for the post-translational processing and modification of proteins. Appropriate cells lines or host systems can be chosen to ensure the correct modification and processing of the foreign protein expressed. In addition, the protein product (e.g., C/EBPβ-1 or C/EBPβ-3) may be co-expressed with a specific kinase which will provide the desired phosphorylation of the protein product.

Expression vectors for use in mammalian cells ordinarily include an origin of replication (as necessary), a promoter located in front of the gene to be expressed, along with any necessary ribosome binding sites, RNA splice sites, polyadenylation site, and possibly transcriptional terminator sequences. The origin of replication may be provided either by construction of the vector to include an exogenous origin, such as may be derived from SV40 or other viral (e.g., polyoma virus, adenovirus, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the gene sequence(s), provided such control sequences are compatible with the host cell systems.

A number of viral based expression systems may be utilized, for example, commonly used promoters are derived from polyoma, Adenovirus 2, and frequently Simian Virus 40 (SV40). The early and late promoters of SV40 virus are particularly useful because both are obtained easily from the virus as a fragment which also contains the SV40 viral origin of replication. Smaller or larger SV40 fragments may also be used, provided there is included the approximately 250 basepair sequence extending from the Hind III site toward the Bg1I site located in the viral origin of replication.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1, E3, or E4) will result in a recombinant virus that is viable and capable of expressing proteins, polypeptides or peptides in infected hosts.

Specific initiation signals may also be required for efficient translation of protein, polypeptide or peptide coding sequences. These signals include the ATG initiation codon and adjacent sequences. Exogenous translational control signals, including the ATG initiation codon, may additionally need to be provided. One of ordinary skill in the art would readily be capable of determining this and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements and transcription terminators.

In eukaryotic expression, one will also typically desire to incorporate into the transcriptional unit an appropriate polyadenylation site (e.g., 5′-AATAAA-3′) if one is not contained within the original cloned segment. Typically, the poly A addition site is placed about 30 to 2000 nucleotides “downstream” of the termination site of the protein at a position prior to transcription termination.

For long-term, high-yield production of a recombinant protein, polypeptide or peptide, stable expression is preferred. For example, cell lines that stably express constructs encoding an protein, polypeptide or peptide by the methods disclosed herein may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines.

A number of selection systems may be used, including, but not limited to, the herpes simplex virus thymidine kinase (tk), hypoxanthine-guanine phosphoribosyltransferase (hgprt) and adenine phosphoribosyltransferase (aprt) genes, in tk⁻, hgprt⁻ or aprt⁻ cells, respectively. Also, antimetabolite resistance can be used as the basis of selection for dihydrofolate reductase (dhfr), that confers resistance to methotrexate; gpt, that confers resistance to mycophenolic acid; neomycin (neo), that confers resistance to the aminoglycoside G-418; and hygromycin (hygro), that confers resistance to hygromycin. Also, puromycin is often used.

Animal cells can be propagated in vitro in two modes: as non-anchorage dependent cells growing in suspension throughout the bulk of the culture or as anchorage-dependent cells requiring attachment to a solid substrate for their propagation (i.e., a monolayer type of cell growth). Non-anchorage dependent or suspension cultures from continuous established cell lines are the most widely used method of large scale production of cells and cell products. However, suspension cultured cells have limitations, such as tumorigenic potential and lower protein production than adherent cells.

Large scale suspension culture of mammalian cells in stirred tanks is a common method for production of recombinant proteins. Two suspension culture reactor designs are in wide use: the stirred reactor and the airlift reactor. The stirred design has successfully been used on an 8000 liter capacity for the production of interferon. Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcorner section of the reactor. The main advantage of this design is the simplicity and lack of need for mechanical mixing. Typically, the height-to-diameter ratio is 10:1. The airlift reactor scales up relatively easily, has good mass transfer of gases and generates relatively low shear forces.

It is contemplated that the proteins, polypeptides or peptides produced by the methods of the invention may be “overexpressed”, i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

5.60 Pharmaceutical Compositions, Administration, and Dosage

In preferred embodiments, the administration of C/EBPβ-1 and/or C/EBPβ-3 comprises introducing either a protein and/or nucleic acid form of C/EBPβ-1 and/or C/EBPβ-3 to a mammalian cell. Preferably, the nucleic acid form expresses C/EBPβ-1 and/or C/EBPβ-3 in the mammalian cell. The administration to the cell can be conducted in vivo, ex vivo, or in vitro. These terms are known in the art. In certain preferred embodiments, the ex vivo approach is used where tissue is removed from the mammal (or an acceptable donor organism), treated, and placed back into the mammal. In certain exemplary embodiments, the in vivo approach is used where the cell is treated directly in the mammal. Naturally, it is preferred that the C/EBPβ-1 and C/EBPβ-3 are administered to the cell in the context of a pharmaceutical formulation or composition.

In certain embodiments, the preferred method of administering C/EBPβ-1 and C/EBPβ-3 isoforms is in combination with an excipient (a pharmaceutically acceptable carrier). The excipient combined with the C/EBPβ-1 and/or C/EBPβ-3 may be administered by any mode or route known and to any cell, tissue, or organ of the mammal. The combination of an pharmaceutically acceptable carrier and the pharmaceutically active ingredient (including, but not limited to, C/EBPβ-1 and/or C/EBPβ-3) is referred to herein as a pharmaceutical formulation. The preferred pharmaceutical formulation is substantially free of a C/EBPβ-2 isoform of C/EBPβ. In certain embodiments, “substantially free of a C/EBPβ-2 isoform” means that a pharmaceutical formulation will contain less than 49% of the C/EBPβ-2 isoform. In more preferred embodiments, “substantially free” means that a pharmaceutical formulation contains less than 5% of the C/EBPβ-2 isoform. In still more preferred embodiments, “substantially free” means that a pharmaceutical formulation will contain less than 1% of the C/EBPβ-2 isoform. The amount of C/EBPβ-2 isoform can be determined by optical density on a silver stained SDS/PAGE gel and is in relation to total protein in a sample.

In certain embodiments, the preferred active ingredient is C/EBPβ-1 (protein or encoding nucleic acid). In certain embodiments, the preferred active ingredient is C/EBPβ-3 (protein or encoding nucleic acid). In certain embodiments, C/EBPβ-1 and C/EBPβ-3 are both preferred active ingredients of the pharmaceutical composition. In certain preferred embodiments, the active ingredient includes novel compositions of C/EBPβ-1 and C/EBPβ-3 disclosed herein including the hybrid EBV/retroviral C/EBPβ-1/C/EBPβ-3 vector modified to prevent expression of C/EBPβ-2 and for enhance expression of C/EBPβ-1 and C/EBPβ-3. Additional active ingredients are also contemplated (for example, anti-tumor factors, such as known forms of tumor treatments that are supplied in addition to the compositions of the present invention).

The particular excipient is not believed to be critical as long as it is compatible with the biological activity of the active ingredient and compatible with administration to the subject (including a cell, mammal, or human). The choice of excipient depends on the type of tumor or cell being treated, the location of treatment, and the active ingredient. A pharmaceutical formulation of liposomes (excipient) and the exemplary hybrid-retroviral/EBV-C/EBPβ-1/C/EBPβ-3 expression vector is highly preferred for certain embodiments. This formulation can be injected into the local tissue or the afferent blood supply for treatment of a tumor or a population of cells, it can be combined with additional inert or carrier ingredients and used as a topical salve (e.g., for treatment of a melanoma or sarcoma), and it can be used with an aerosolization device for inhalation (e.g., to treat a bronchiopulmonary tumor).

As mentioned, the choice of excipient depends on the type, location, and nature of the tumor; as well as, the route and mode of administration. The choice of pharmaceutically acceptable carrier can be made by one with skill in the art, such as the treating physician. The liposome/adenoviral formulation and additional recommended carriers, formulations, and modes of administration are described below.

Pharmaceutical compositions of the present invention will have an effective amount of a gene or peptide for therapeutic administration in combination with a pharmaceutically acceptable carrier. The gene or peptide may be dissolved or dispersed in the carrier and the carrier may be as simple as water. Although purified and sterile water is preferred and the addition of salts, pH buffers, and preservatives may be desired.

As used herein, “pharmaceutically acceptable carrier” includes any and all solvents, dispersion media, coatings, antibacterial and antifungal agents, isotonic and absorption delaying agents and the like. “Pharmaceutically acceptable carrier” also includes water, and water plus buffers and/or salts. The use of such media and agents for pharmaceutical active substances is well known in the art. Except insofar as any conventional media or agent is incompatible with the active ingredients, its use in the therapeutic compositions is contemplated. Thus, the numerous examples of pharmaceutically acceptable carriers that are provided herein, are provided by way of example and are not meant to limit the scope of the present invention. Supplementary active ingredients, such as other anti-tumor agents, can also be incorporated into the compositions. These may include, but are not limited to: radiotherapeutics, chemotherapeutics, hormone therapy, or other biological therapies. Also, compounds that are found to act synergistically with agents of the present invention may be used in combination or incorporated into the pharmaceutical compositions. The present invention may also be performed in combination with surgery.

In one example, the present compositions and methods may be used to render an unresectable tumor resectable. A tumor may be unresectable due to size, location, dissemination, etc. The compositions of the present invention can be used to reduce the size, eliminate the tumor in an unresectable location, or treat a metastatic portion of a cancer which has disseminated. Thus, the compositions and methods of the present invention can be used in combination with surgical resection of a tumor, or portion of a tumor, at any point before, during, or after resection, or some combination thereof. The compositions and methods of the present invention can be used also as a inhibitor to the recurrence of a tumor, including a metastatic tumor.

The expression vectors and delivery vehicles of the present invention may include classic pharmaceutical preparations. Administration of these compositions according to the present invention will be via any common route so long as the target tissue is available via that route. This includes, but is not limited to: oral, dermal, nasal, buccal, rectal, vaginal or topical. Administration may be by orthotopic, intradermal, subcutaneous, intramuscular, intraocular, intraperitoneal or intravenous injection. An exemplary route of administration is intratumoral. Such compositions would normally be administered as pharmaceutically acceptable compositions, described supra. In certain preferred embodiments, pharmaceutical formulations of the present invention are directly injected into a tumor or among a population of cells. In the case of intratumoral injection, it is preferred that the pharmaceutical formulation is injected into the tumor, optionally multiple times, optionally multiple times spaced about 5 millimeters apart over the area of the tumor.

In embodiments wherein a tumor is identified, the tumor can be in and treated in any body component or in multiple body components including, but not limited to the: adipose, bladder, bone, brain, central nervous system, cartilage, cervix, eye, fallopian tube, heart, intestine, joint, kidney, liver, lung, lymphoid, muscle, ovary, pancreas, peripheral nervous system, peritoneum, prostate, skin, spleen, stomach, tendon, testicle, uterus, and vasculature.

In embodiments wherein a tumor is identified, the tumor can have originated from cells in any body component or in multiple body components including, but not limited to the: adipose, bladder, bone, brain, central nervous system, cartilage, cervix, eye, fallopian tube, heart, intestine, joint, kidney, liver, lung, lymphoid, muscle, ovary, pancreas, peripheral nervous system, peritoneum, prostate, skin, spleen, stomach, tendon, testicle, uterus, and vasculature. In certain embodiments the above body components are treated when a tumor has not been identified or when a subject has not been examined for a tumor. The purposes of such treatment with compositions and methods of the present invention include, but are not limited to: the inhibition of tumorigenesis, the inhibition of cellular proliferation, the stimulation of cellular differentiation, and the potentiation of cell death.

The vectors of the present invention are advantageously administered in the form of injectable compositions either as liquid solutions or suspensions; solid forms suitable may be solubilized or suspended in liquid prior to injection. These preparations also may be emulsified. In certain embodiments, a typical composition comprises about 50 mg or up to about 100 mg of human serum albumin per milliliter of phosphate buffered saline. Other pharmaceutically acceptable carriers include aqueous solutions, salts, preservatives, buffers and the like. Examples of non-aqueous solvents are propylene glycol polyethylene glycol. vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well known parameters.

Additional formulations are suitable for oral administration. Oral formulations include such typical excipients as; for example, pharmaceutical grades of mannitol, lactose, starch, magnesium stearate, sodium saccharine, cellulose, magnesium carbonate, and the like. The compositions take the form of solutions. suspensions tablets, pills, capsules, sustained release formulations or powders. When the route is topical, the form may be a cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

All the essential materials and reagents required for treating a tumor, inhibiting tumor cell proliferation, inhibiting cellular proliferation in a population of cells, stimulating cellular differentiation, potentiating cell death, and inducing cell death; may be assembled together in a kit. When the components of the kit are provided in one or more liquid solutions, the liquid solution preferably is an aqueous solution, with a sterile aqueous solution being particularly preferred. Containers for the components may include an inhalant, syringe, pipette, eye dropper, or other apparatus, from which the formulation may be applied to an infected area of the body, such as the lungs, injected into a mammal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container within the kit. The kits of the invention may also include an instruction sheet defining administration of C/EBPβ-1 and/or C/EBPβ-3 polypeptide therapy and/or gene therapy and the pharmaceutical indications of the kit components.

The kits of the present invention also will typically include a vessel for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of a mammal. Such an instrument may be an inhalant, syringe, pipette, forceps, measured spoon, eye dropper or any such medically approved delivery vehicle.

Parenteral administration, such as intravenous or intramuscular injection, is an exemplary method of delivering the anti-tumor agents of the present invention to many tumor types and cell populations. Administration of polypeptides and nucleic acids by injection for the treatment of disease is described in U.S. Pat. No. 5,580,859 Feigner et al., incorporated herein by reference. Alternatively non-parenteral administration may be desired, including: oral administration; time release capsules; and any other form known in the art, including cremes, lotions, mouthwashes, inhalants and the like. In one example, gene therapy using the intestine is described in U.S. Pat. No. 5,821,235 to Henning et al., incorporated herein by reference.

The active compounds of the present invention will often be formulated for parenteral administration, e.g., formulated for injection via the intravenous, intramuscular, subcutaneous, and intraperitoneal routes. Typically, such compositions can be prepared as injectables, either as liquid solutions or suspensions; solid forms suitable for preparation of solutions or suspensions upon the addition of a liquid prior to injection may be desired; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations may contain a preservative to inhibit or prevent the growth of microorganisms.

Pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions can be used. It is preferred that the form is sterile and that it is fluid to the extent that it can be aspirated into a syringe. It should be stable under the conditions of manufacture and storage and should be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof and vegetable oils. The proper fluidity can be maintained, for example, by the use of a coating, such as lecithin, by the maintenance of the required particle size in the case of dispersion and by the use of surfactants. The inhibition or prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as needed, followed by filter sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in cremes and lotions. These forms may be used for treating skin-associated diseases, such as various sarcomas.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. The sterile aqueous media which can be employed will be known to those of skill in the art in light of the present disclosure. For example, one dosage could be dissolved in 1 milliliter of isotonic NaCl solution and either added to about 1 liter of hypodermoclysis fluid or injected at the proposed site of infusion, (see for example, “Remington's Pharmaceutical Sciences” 15th Edition, pages 1035-1038 and 1570-1580, incorporated herein by reference). Some variation in dosage will necessarily occur depending on the condition of the subject being treated. The person responsible for administration will, in any event, determine the appropriate dose for the individual subject.

Targeting of cancerous tissues may be accomplished in any one of a variety of ways. Plasmid vectors and retroviral vectors, adenovirus vectors, and other viral vectors all present methods by which to target human cancers. The inventor anticipates particular success for the use of liposomes to transfer polynucleotides and polypeptides of the present invention into tumor cells including cancer cells. In one of the first series of clinical phase to be performed, DNA encoding C/EBPβ-1 and/or C/EBP3 will be mixed with liposomes, and this DNA/liposome complex will be injected into patients with certain forms of advanced stage breast cancer. Intravenous injection into the afferent blood supply to the organ can be used to direct the gene to most or all cells of the breast and/or tumor. Directly injecting the liposome complex into the proximity of a cancer can also provide for targeting of the complex with some forms of cancer. For example, cancers of the ovary can be targeted by injecting the liposome mixture directly into the peritoneal cavity of patients with ovarian cancer. Of course the potential for liposomes that are selectively taken up by a population of cancerous cells exists, and such liposomes will also be useful for targeting the gene.

Those of skill in the art will recognize that the best treatment regimens for using compositions of the present invention, including C/EBPβ-1 and/or C/EBPβ-3, to suppress tumors can be straightforwardly determined. This is not a question of experimentation, but rather one of optimization which is routinely conducted in the medical arts. The in vivo studies in nude mice provide a starting point from which to begin to optimize the dosage and delivery regimes. The frequency of injection will initially be once a week. However, this frequency might be optimally adjusted from one day to every two weeks to monthly, depending upon the results obtained from the initial clinical trials and the needs of a particular patient. Human dosage amounts can initially be determined by extrapolating from tests with other anti-tumor biological therapeutics; for example, U.S. Pat. No. 5,922,688 to Hung et al., incorporated herein by reference. Accordingly, approximately 15 μg of C/EBPβ-1 and/or C/EBPβ-3 DNA per 50 kg body weight is desirable. Based on this, a 50 kg woman would receive a dose of approximately 15 mg of C/EBPβ-1 and/or C/EBPβ-3 DNA per treatment. In certain embodiments it is envisioned that this dosage may vary from between about 100 μg/50 kg body weight to about 5 μg/g body weight; or from about 90 μg/50 kg body weight to about 10 μg/g body weight or from about 80 μg/50 kg weight to about 15 μg/g body weight; or from about 75 μg/50 kg body weight to about 20 μg/g body weight; or from about 60 μg/50 kg body weight to about 30 μg/g body weight about 50 μg/50 kg body weight to about 40 μg/g body weight. In certain embodiments this dose may be about 0.015, 0.15, 0.5, 1, 2, 3, 5, 8, 10 15, or 20 μg/50 kg of body weight. Of course, these dosage amounts may be adjusted upward or downward, as is routinely done in such treatment protocols, depending on the results of the initial clinical trials and the needs of a particular patient. Dosage amounts may be adjusted upward or downward by any amount determined to be needed including 10 fold, 100 fold, and 1000 fold.

5.61 Administration by Transfection

The term “transfection” generally refers to the introduction of a nucleic acid into a eukaryotic cell. The present invention also contemplates the transfer or “transfection” of polypeptide/protein forms of C/EBPβ-1 and C/EBPβ-3 into mammalian cells. In many the instances, the methods used for nucleic acid transfection are easily adopted for the transfer of proteins and are known in the art (see, e.g., Gene Transfer and Expression Protocols (Methods in Molecular Biology, VOL 7) (1991) E. J. Murray (ed.) Humana Press; M. Kriegler, Gene Transfer and Expression: A Laboratory Manual (1991) Oxford University Press, each reference incorporated herein by reference). In addition, there are numerous commercially available transfection kits (e.g., Stratagene, Invitrogen, Roche, and the like). Transfection techniques being utilized in vivo and ex vivo, are disclosed in U.S. Pat. No. 5,858,784 to R. J. Debs et al., incorporated herein by reference. Transfection techniques are particularly useful for the transfer of nucleic acid compositions of the present invention into cells wherein transduction by viral-based expression vectors is not employed. Although, these techniques may be used in combination with viral-based vector transduction.

5.62 Lipid Mediated Transfer

Liposome mediated transfection is an exemplary method of introducing C/EBPβ-1 and/or C/EBPβ-3 polypeptide or polynucleotide compositions into a cell for treatment. Liposome and lipid based methods are readily known to those of skill in the art and numerous kits are available commercially (see e.g., Liposome Technology: Liposome Preparation and Related Techniques (1992) G. Gregoriadis (ed.) CRC Press, incorporated herein by reference; U.S. Pat. No. 5,279,833 to Rose, incorporated herein by reference; U.S. Pat. No. 5,567,433 to Collins, incorporated herein by reference; U.S. Pat. No. 4,515,736 to Deamer, incorporated herein by reference; Felgner et al., (1987) Proc. Nat. Acad. Sci., USA 84:471-477, incorporated herein by reference; and Gao et al (1991) Biochem. Biophys. Res. Comm. 179:280-285, incorporated herein by reference).

Currently, gene delivery with cationic lipids is currently the most clinically developed approach to gene therapy (Gene Therapy of Cancer (1999) Lattime et al., (eds.) Academic Press, Chapter 20, incorporated herein by reference). In general, cationic lipids are synthetically manufactured and can be readily mixed with any polynucleotide or polypeptide desired and form linkages based on non-covalent charge-charge interactions; although, covalent linkage is possible also. Numerous cationic lipids are available commercially and can be used in conjunction with the present invention for gene delivery (including in vivo, in vitro, and ex vivo). One of the biggest advantages to using cationic or liposome mediated gene transfer is that the non-infectious expression vectors described herein are not immunogenic when administered to a mammal including a human. This is especially true when compared to adenoviral-based expression systems which may be the most immunogenic (although useable) embodiment described herein.

Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. One such commercially available liposomal transfection reagent is Lipofectamin™ (DOTMA:DOPE by Gibco-BRL).

In certain embodiments of the present invention, the liposome may be complexed with a hemagglutinating virus (HVJ). This has been shown to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-1. In other embodiments, the delivery vehicle may comprise a ligand and a liposome to target the liposome to particular cell types or tissues

U.S. Pat. No. 5,059,421 to Loughrey et al., incorporated herein by reference; describes a general method of attaching protein molecules to liposomes to achieve well-characterized and sized protein-liposome conjugate systems for inter-changeable targeting applications. This pharmaceutical liposomal composition can be targeted to essentially any cell type or tissue, if desired. U.S. Pat. No. 4,885,172 to Bally et al., incorporated herein by reference; describes compositions and methods for storing liposomes including targeted liposomes and the loading of the such liposomes on an “as needed” basis. U.S. Pat. No. 5,851,818 to Huang et al., incorporated herein by reference; discloses improved methods for preparing nucleic acid/liposome complexes including selection of the working medium and liposome lipid to nucleic acid ratios. U.S. Pat. No. 5,279,883 to Rose, incorporated herein by reference; describes liposomal transfection of nucleic acids into animal cells. U.S. Pat. No. 5,225,212 to Martin, incorporated herein by reference; describes a liposome composition for extended release of a therapeutic compound into the bloodstream and methods for use thereof.

5.63 Membrane Transport Sequence (MTS) Mediated Transfer

In certain embodiments, a membrane transport sequence (MTS) can function as an agent for the administration of a composition of the present invention, including, but not limited to C/EBPβ-1 and/or C/EBPβ-3, to a cell. It is demonstrated that when a cell is contacted with a composition linked to an MTS, the entire MTS linked composition translocates through the cytoplasmic membrane of a cell (U.S. Pat. No. 5,807,746 to Lin et al., incorporated herein by reference and U.S. Pat. No. 5,877,282 to Nadler et al., incorporated herein by reference). Two functional MTS sequences are provided in the Sequence Listings (SEQ ID NO:12 and SEQ ID NO:13). Additional functional MTS sequences are described in U.S. Pat. No. 5,962,415 to Nadler, incorporated herein by reference.

The MTS can be combined with C/EBPβ-1 and C/EBPβ-3 chemically utilizing the carboxy and amino groups on the proteins or by molecular cloning of an MTS encoding DNA sequence into the C/EBPβ-1 and/or C/EBPβ-3 expression vector to form a fusion gene with subsequent expression of a fusion protein. The fusion protein may subsequently be expressed in vitro or in vivo. A fusion gene or fusion protein is one in which two or more sequences which are not combined in nature are combined by the hand of man. A similar term is “chimeric”. The MTS-C/EBPβ-1 chimera or MTS-C/EBPβ-3 chimera may include a linker sequence if desired.

5.65 Electroporation, Calcium Phosphate, and Particle Bombardment

In certain embodiments a composition of the present invention is introduced into a cell via electroporation. Electroporation involves the exposure of a suspension of cells and the composition to a high-voltage electric discharge (see U.S. Pat. No. 4,956,288 to Barsoum, incorporated herein by reference). Transfection of nucleic acids and proteins into eukaryotic cells using electroporation is quite successful and known in the art.

In certain embodiments a composition of the present invention is introduced into a cell using calcium phosphate precipitation. Transfection with calcium phosphate is described in U.S. Pat. No. 5,633,156 to Wurm et al., incorporated herein by reference. Human KB cells have been transfected with adenovirus 5 DNA using this technique (Graham et al., (1973) Virology 52:456-467, incorporated herein by reference).

In an alternative embodiment, a composition of the present invention is introduced into a cell by methods that include particle bombardment. This method depends on the ability to accelerate nucleic acid-coated microprojectiles to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed. and still obtain a like or similar result without departing from the spirit and scope of the present invention.

### EXAMPLE 1

### Expression of C/EBPβ Isoforms

Cytoplasmic and nuclear fractions are prepared from cultured human mammary epithelial cells (HMEC), resolved by 10% SDS-PAGE, and transferred to a nylon membrane. Whole cell extracts are prepared from any cell type being examined, including: MDA468, MDA 231, MCF7-neo, MCF7-218, MCF7, T47D, MCF10A, and BT474 immortalized cultured cell lines which are derived from mammary carcinomas; normal breast cells from reduction mammoplasty; and primary mammary tumor cells. The extracts are transferred to a nylon membrane in the same manner. The membranes are probed with affinity purified antibody to the C-terminal C/EBPβ peptide (available from Santa Cruz; see FIG. 1) or with an N-terminal C/EBPβ-1 specific antibody, supra, and detected with Supersignal CL-HRP substrate (Pierce) according to the manufacture's instructions. Positions of molecular weight markers and C/EBPβ protein are shown.

As shown in FIG. 6A, the expression product of C/EBPβ-1 in HMECs is essentially confined to the nuclear compartment of the cells. C/EBPβ-3 is also essentially confined to the nuclear compartment for this cell line. C/EBPβ-2 expression, however, is essentially observed in the cytoplasmic compartment of HMECs.

The data in FIG. 6A, demonstrate the following for C/EBPβ isoform expression in the whole cell extract of the cell lines derived from mammary carcinomas: C/EBPβ-1 expression is undetectable; C/EBPβ-2 is expressed in high levels; and the level of C/EBPβ-3 expression is variable among the cell lines, but generally less than the level of C/EBPβ-3 expression observed in HMECs.

Most of the cell lines represented in FIG. 6A, are rapidly proliferating in cell culture with a doubling time of about 24 hours in the mammary carcinoma cell line, and somewhat more than 24 hours in the HMECs. The BT474 cells, however, are slow growing. C/EBPβ-2 expression is observed in all proliferating cell lines tested. In the HMEC cell line, which is not capable of producing tumors after implantation in nude mice, the C/EBPβ-2 expression is compartmentalized in the cytoplasm.

### EXAMPLE 2

### Construction of Epitope Tagged C/EBPβ-1 Retroviral Vector

A hybrid Epstein-Barr virus (EBV)/retroviral vector construct, LZRSpBMN-Z, was provided by Gary P. Nolan (Stanford University, California, USA). The LZRSpBMN-Z is described in U.S. Pat. No. 5,830,725 to Nolan et al., incorporated herein by reference. The LZRSpBMN-Z vector features episomal replication which enables production of high titer virus and high efficiency gene transfer. In addition to a murine retroviral backbone with a polylinker region to facilitate insertion of cDNAs, the LZRSpBMN-Z vector contains the Epstein-Barr virus Nuclear Antigen (EBNA) gene, EBV origin of replication and nuclear retention sequences (oriP), and a puromycin resistance gene. The nuclear replication and retention function of this vector allow for rapid establishment of recombinant retroviral producer DNA as stable episomes within human retroviral packaging cell lines. Episomes are maintained at approximately 5-20 copies per cell for up to 2-3 months, given selection for puromycin resistance, resulting in high viral titers. The retroviral backbone in this vector consists of full-length Moloney murine leukemia virus (MoMLV) long terminal repeat (LTR) and extended Ψ packaging sequences derived from the MFG series of retroviral vectors developed by Mulligan and colleagues.

To obtain LZRS vector encoding epitope-tagged C/EBPβ-1, the β-galactosidase gene encoded by the prototype LZRSpBMN-Z vector is excised and replaced with his-tagged C/EBPβ-1 fragments to generate a pLZRShisC/EBPβ-1 vector. The construction of the pLZRShisC/EBPβ-1 vector is completed in several steps (FIGS. 9A-9D). First a CMV-NFIL6 vector is digested with Sal I, incubated at 4C with DNA Polymerase I to generate blunt ends, and digested with Eco RI to release a 1,045 basepair fragment containing C/EBPβ-1 from CMV-NFIL6. In this case the CMV-NFIL6 vector was obtained from S. Akira. However, a clone of C/EBPβ-1 can be obtained using standard methods known to one with skill in the art from a cDNA library or genomic DNA. Next, the 1,045 basepair fragment is inserted into the pRSETC vector (available from Invitrogen, San Diego, Calif., USA) for mutagenesis and to acquire the polyhistidine epitope tag which is included in the pRSETC vector (FIG. 9A). The PRSETC vector is digested with Hind III, incubated at 4C with DNA Polymerase I, and digested by Eco RI, and the 1,045 basepair C/EBPβ-1 fragment is ligated into place forming the pRSETC-C/EBPβ (which is also called pRSETC-NFIL6) (FIG. 9A). Mutagenesis is conducted by replacing a 106 basepair Bgl II to Msc I fragment of the pRSETC-C/EBPβ-1 vector with synthetic oligos (commercially available) shown in FIG. 9B. The top strand oligo is listed in SEQ ID NO:15. The bottom strand oligo is listed in SEQ ID NO:16. The mutant residues in the top strand oligonucleotide creating the Kozak sequence are underlined in FIG. 9B and run from about position 293 to 302. The mutant residues eliminating the ATG for C/EBPβ-2 expression are underlined also in FIG. 9B at approximately positions 368 and 369. The mutations can be compared to the wild-type C/EBPβ sequence for this region as shown in FIG. 9B, also. FIG. 9C shows that cloning of the epitope tagged C/EBPβ-1 with Kozak and C/EBPβ-2 elimination mutations from pcDNA3.1HisAC/EBPβ-1 into pLZRSpBMN-Z forming the pLZRS-His-C/EBPβ-1 vector. FIG. 9D shows a diagram of the LZRS vector with an epitope-tagged C/EBPβ-1 insert (pLZRShisC/EBPβ-1). Control vector would remain unchanged and contain a β-galactosidase encoding sequence. To ensure selective expression of C/EBPβ-1, a perfect Kozak sequence is created around the first ATG and the 2^{nd} in-frame methionine in C/EBPβ is mutated to glycine by site-directed mutagenesis techniques.

Helper-free retrovirus is obtained by transfecting the pLZRShisC/EBPβ-1 vector, or control LZRSpBMN-Z vector encoding β-galactosidase, into a 293T-based amphotropic packaging cell line termed φnx-ampho (provided by G. Nolan). These methods are known to those of skill in the art. Briefly, 1 μg of pLZRShisC/EBPβ-1 vector or 1 μg of LZRSpBMN-Z vector is transfected into the φnx-ampho cells using GenePorter liposome reagent according to the manufacture's directions. Transfected cells are maintained in puromycin to select for episomal maintenance of the transfected vector.

Three weeks prior to performing transfections, (Ψ)nx-ampho cells are reselected in the presence of diphtheria toxin and hygromycin B to increase envelope and gag-pol expression. Packaging cells are then transfected by standard calcium phosphate procedures, and viral supernatants (in culture medium) are harvested at 48 hours post-transfection, clarified, and stored frozen at 800C. Cells are then trypsinized and replated in medium containing puromycin (to select for episomal maintenance of the LZRS-based construct). Upon reaching 70% confluence, cells are placed in puromycin free medium for 24 hrs prior to harvesting virus as before. This procedure is carried out for production and collection of viral stocks for up to 3 weeks post-transfection. For general reference, see, Nolan et al. (1998) Expression vectors and delivery systems. Curr. Opin. Biotechnol. 9, 447-450 and Grignani et al. (1998) High efficiency gene transfer and selection of human hematopoietic progenitor cells with a hybrid EBV/retroviral vector expressing the green fluorescence protein Cancer Res. 58, 14-19; each reference incorporated herein by reference.

### EXAMPLE 3

### Construction of Epitope Tagged C/EBPβ-3 Retroviral Vector

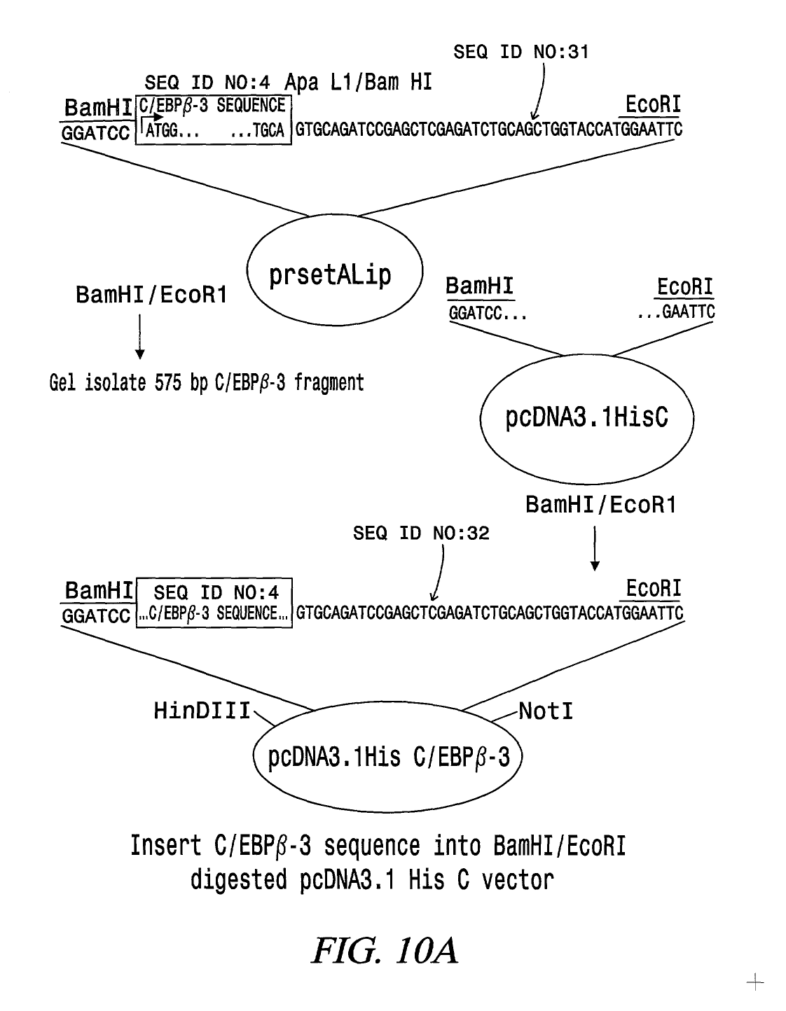

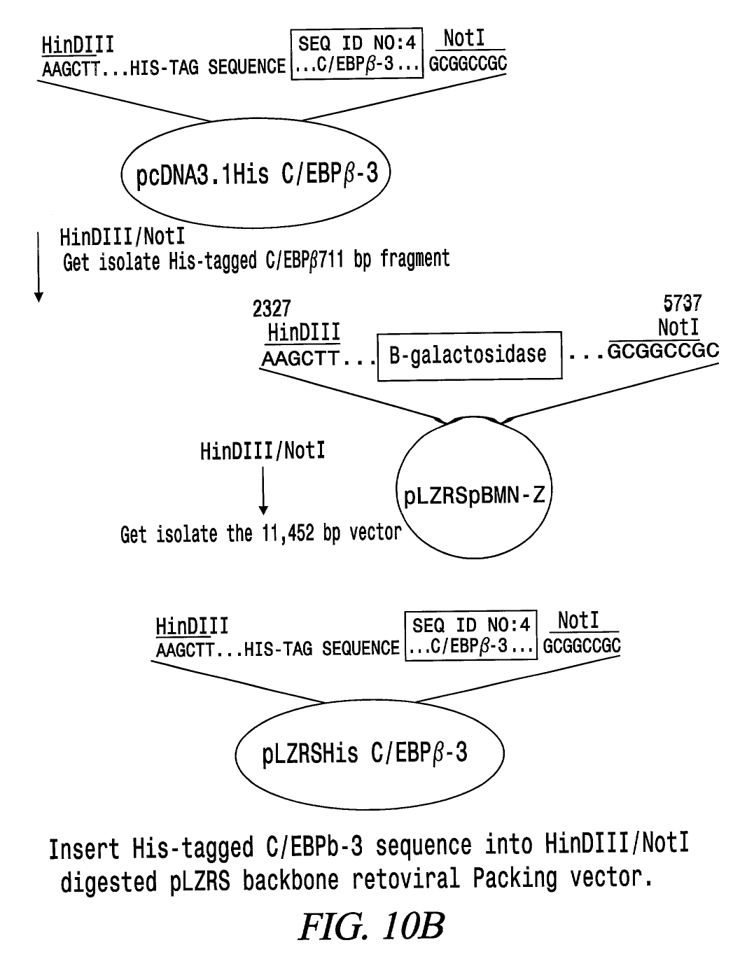

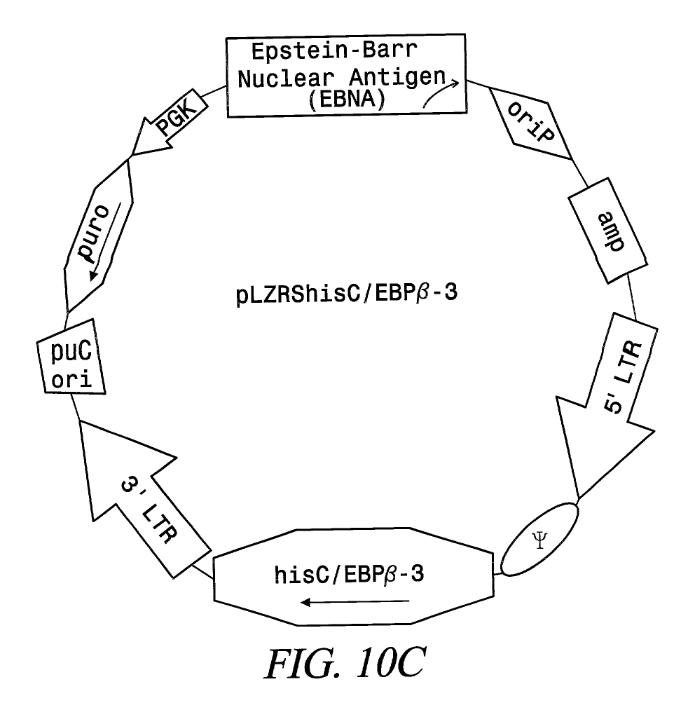

To obtain LZRS vector encoding epitope-tagged C/EBPβ-3, prsetALip plasmid (including the C/EBPβ-3 sequence) is digested with Bam HI and Eco RI restriction endonucleases followed by gel electrophoresis and isolation of a 575 basepair C/EBPβ-3 fragment. The C/EBPβ-3 fragment is ligated into a similarly digested (Bam HI and Eco RI) pcDNA3.1HisC vector to generate pcDNA3.1HisC/EBPβ-3 plasmid (FIG. 10A). This vector is then digested with Hin DIII/Not I restriction endonucleases followed by gel electrophoresis and isolation of a 711 basepair His-tagged C/EBPβ-3 fragment. The pLZRSpBMN-Z plasmid is digested with Hin DIII/Not I also. A 11,452 basepair fragment is isolated and ligated to the 711 basepair His-tagged C/EBPβ-3 fragment to generate pLZRS-His-C/EBPβ-3 (FIG. 10B). Again the control vector contains the β-galactosidase gene. A diagram of the pLZRS-His-C/EBPβ-3 vector is shown in FIG. 10C.

Helper-free retrovirus is obtained by transfecting the pLZRShisC/EBPβ-3 vector, or control LZRSpBMN-Z vector encoding β-galactosidase, into a 293T-based amphotropic packaging cell line termed φnx-ampho (provided by G. Nolan). These methods are known to those of skill in the art. Briefly, 1 μg of pLZRShisC/EBPβ-3 vector or 1 μg of LZRSpBMN-Z vector is transfected into the φnx-ampho cells using GenePorter liposome reagent according to the manufacture's directions. Transfected cells were maintained in puromycin to select for episomal maintenance of the transfected vector. High titre virus is collected as described in Example 2.

Compared to the transfection with the control prototype LZRSpBMN-Z vector, packaging cells transfected with pLZRShisC/EBPβ-3 stopped growing over a period of several weeks (these data are not shown). The decrease in proliferation of the pLZRShisC/EBPβ transfected cells is associated with a decline in retroviral production. This is evidence of the growth inhibitory properties of the C/EBPβ-3 gene product. High titer, helper free C/EBPβ-3 retrovirus can be collected during the first week after transfection, however.

### EXAMPLE 4

### Construction of Epitope Tagged C/EBPβ-1 Recombinant Adenoviral Vector

A recombinant adenovirus encoding vector, referred to herein as pGEM-RecA vector, was provided by Joe Nevins of Duke University, North Carolina. The C/EBPβ-1 gene is cloned into the pGEM-RecA vector along with a triple copy of the influenza hemagglutinin (HA) epitope tag forming the Ad-HA-C/EBPβ-1 vector, using standard techniques known to one with skill in the art. In the pGEM-RecA system, the insert, C/EBPβ-1 in this case, is placed under the control of a CMV promoter.

Samples of 293 cells are transfected with a mixture of linearized Ad-HA-C/EBPβ-1 vector and Xba I and Cla I cut adenovirus type 5 DNA (provided by Nevins) and the linearized vector undergoes in vivo recombination with the host cell genomic DNA. Lysates of the transfected 293 cells are prepared 5 days post-transfection and potential recombinant viruses are isolated by plaque assays on non-transfected 293 cells. After plaque purification, recombinants vectors are detected by Southern blotting methods, known to one with skill in the art. Ad-HA-C/EBPβ-1 viral stocks are obtained by transduction of monolayer culture of 293 cells and preparation of crude cell lysates. Recombinant virus is purified by CsCl density gradient centrifugation and titered as known to one with skill in the art (see, e.g. DeGregori, et al. (1995) MCB 10:5846-5847, incorporated herein by reference). As a control, MbAd2 virus, containing only pGEM-RecA vector without a cDNA insert is used.

### EXAMPLE 5

### Construction of Epitope Tagged C/EBPβ-3 Recombinant Adenoviral Vector

A recombinant adenovirus encoding vector, referred to herein as pGEM-RecA vector, was provided by Joe Nevins of Duke University, North Carolina. The C/EBPβ-3 gene is cloned into the pGEM-RecA vector along with a triple copy of the influenza hemagglutinin (HA) epitope tag forming the Ad-HA-C/EBPβ-3 vector, using standard techniques known to one with skill in the art. In the pGEM-RecA system, the insert, C/EBPβ-3 in this case, is placed under the control of a CMV promoter.

Samples of 293 cells are transfected with a mixture of linearized Ad-HA-C/EBPβ-3 vector and Xba I and Cla I cut adenovirus type 5 DNA (provided by Nevins) and the linearized vector undergoes in vivo recombination with the host cell genomic DNA. Lysates of the transfected 293 cells are prepared 5 days post-transfection and potential recombinant viruses are isolated by plaque assays on non-transfected 293 cells. After plaque purification, recombinants vectors are detected by Southern blotting methods, known to one with skill in the art. Ad-HA-C/EBPβ-3 viral stocks are obtained by transduction of monolayer culture of 293 cells and preparation of crude cell lysates. Recombinant virus is purified by CsCl density gradient centrifugation and titered as known to one with skill in the art (supra). As a control, MbAd2 virus, containing only pGEM-RecA vector without a cDNA insert is used.

### EXAMPLE 6

### C/EBPβ-1, C/EBPβ-3, and Variants as Anti-Tumor Agents

A dysregulation of the biological activity of one or more C/EBPβ isoform is shown to result in tumorigenesis as described above. This leads to testable methods for treating tumors using C/EBPβ-1, C/EBPβ-3, or a combination of both C/EBPβ-1 and C/EBPβ-3. These same methods apply to testing the biological functional equivalence of C/EBPβ-1 and C/EBPβ-3 sequences that are mutated, altered, modified, variant, polymorphic, etc. It is specifically contemplated that the same methods apply to testing C/EBPβ-1, C/EBPβ-3, and variants for activity in treating a population of cells to inhibit proliferation and tumorigenesis and to stimulate differentiation and cell death. These latter methods are implied although not necessarily stated when referring to “treating tumors”.

### EXAMPLE 7

### Transduction of Human Breast Cancer Cell Lines

Human breast cancer cell lines that are estrogen receptor positive (BT474, T47D, and MCF-7) and estrogen receptor negative (MDA-468, MDA-231) are transduced in culture with viral stocks of pLZRShisC/EBPβ-1, pLZRShisC/EBPβ-3, or Ad-HA-C/EBPβ-3 using methods known to one with skill in the art. A property of the BT474 cell line is the overexpression of the HER-2/erbB2 receptor. A Property of the MDA-468 cell line is the overexpression of the epidermal growth factor (EGF) receptor. The relative expression of C/EBPβ-3 in cultures of each cell line are shown in FIG. 6A. As seen in FIG. 6A, C/EBPβ-3 expression is roughly 25% that of C/EBPβ-2 expression in the MDA468 cell line. Also seen in FIG. 6A, is that the relative expression of C/EBPβ-3 to C/EBPβ-2 is different in each cell line, in order from highest C/EBPβ-3 expression to lowest is: MDA468, MCF7-neo, BT474, MCF7-218, and MDA 231. The MCF7 and T47D cell lines do not express detectable amounts of C/EBPβ-3 as seen in FIG. 6A.

The variable expression of C/EBPβ-3 relative to C/EBPβ-2 in each cell line allows for the determination of the effect of C/EBPβ-3 overexpression on cell growth and relationship of C/EBPβ-3 overexpression to estrogen receptor status, erbB2 receptor status, EGF receptor status, and to the C/EBPβ-3 levels normally in the cells.

The following cell types will be transduced or sham-transduced with the retroviral and adenoviral vectors detailed above: HMECs (normal control), the panel of mammary carcinoma cell lines (MDA468, MDA 231, MCF7-neo, MCF7-218, MCF7, T47D, and BT474), and cells derived from primary breast tumors. Such viral transduction techniques are known in the art.

To ascertain the fraction of cells transduced with the present chimeric viral vectors (LZRS-his-C/EBPβ-3, LZRS-his-C/EBPβ-3, adeno-C/EBPβ-1, and adeno-C/EBPβ-3), immunofluorescence microscopy is performed two days post-transduction using monoclonal antibodies to the epitope tag of each vector. These antibodies are widely available commercially. Non-transduced cells or immunofluorescence of transduced cells in the absence of primary antibody will be used to control for non-specific reactivity. For subcellular localization of the tagged protein, a confocal laser scanning microscope is used to view immunofluorescence. Western blotting is performed to assess the overall level of tagged C/EBPβ-1 or C/EBPβ-3 expression relative to endogenous C/EBPβ isoform expression in transduced cell populations. For experimental purposes only in determining growth potential, the transduction is judged successful if greater than 90% of the transduced cells stain positive for tagged C/EBPβ-1 or C/EBPβ-3 in the nucleus. If the proportion of transduced cells is not sufficient after a single transduction, the exposure of the cells to the viral vector is repeated until greater than 90% of the cells stain positive. This is to provide a uniform base from which to make experimental comparisons. Transduction efficiency does not necessarily need to be determined along with therapeutic efficiency, but can be determined by the treating physician.

### EXAMPLE 8

### Growth Potential: Fraction of Cells in S Phase

The fraction of cells in S phase of the cell cycle is determined by fluorescent activated cell sorting (FACS) analysis of cells independently transduced with LZRS-his-C/EBPβ-3, LZRS-his-C/EBPβ-3, adeno-C/EBPβ-1, and adeno-C/EBPβ-3 and compared to cells transduced with parental viral vector without a C/EBPβ-1 or a C/EBPβ-3 insert and to non-transduced cells. It is expected that the number of tagged-C/EBPβ immuno-positive cells in S phase will be significantly lower than the control-transduced or non-transduced randomly growing cells as a result of a block to cellular proliferation. FACS analysis will also yield a determination of the cell cycle phase for growth arrest induced by the overexpression of C/EBPβ-1 and C/EBPβ-3. Cells to be examined include, but are not limited to: HMECs (normal control), the panel of mammary carcinoma cell lines (MDA468, MDA 231, MCF7-neo, MCF7-218, MCF7, T47D, and BT474), and cells derived from primary breast tumors.

It is also contemplated that the expression of C/EBPβ-3 will induce cell death in the cells. If cell death is induced, then the living cells may have a normal S phase distribution until cell death occurs whereby the cells are eliminated from the population. The apoptotic potential of the cells will be determined by a TUNEL assay which is known in the art. Apoptotic nuclei in fixed cells will be visualized by end-labeling oligonucleosomal DNA in situ using the TdT In Situ Non-isotopic End Labeling Kit from Amersham/Pharmacia, according to the manufacture's instructions. The methods of Example 8 can be used to determine biological activity of compositions of the present invention including biologically functional variants.

### EXAMPLE 9

### Growth Potential: Doubling Time

The rate at which a population of cells doubles in number is a common method for determining the relative growth potential of cells grown in culture. Cells to be examined include, but are not limited to: HMECs (normal control), the panel of mammary carcinoma cell lines (MDA468, MDA 231, MCF7-neo, MCF7-218, MCF7, T47D, and BT474), and cells derived from primary breast tumors. It is expected that the expression of C/EBPβ-1 or C/EBPβ-3 (or overexpression) in these cells will result in a significant increase in the doubling time. Cancer derived cells are expected to show the most dramatic increase in doubling time. HMECs already have a relatively long doubling time. Doubling time can be used to assay for biological activity of a composition of the present invention including biologically functional equivalents.

### EXAMPLE 10

### Growth Potential: Colony Forming Ability and Assay

The ability of cells to form colonies has long been used to assess growth potential and can be used to identify a biological activity of a composition of the present invention including biologically functional equivalents. Comparisons will be made between cells including, but not limited to: HMECs (normal control), the panel of mammary carcinoma cell lines (MDA468, MDA 231, MCF7-neo, MCF7-218, MCF7, T47D, BT474, and MCF10A), and cells derived from primary breast tumors which are separately transduced with LZRS-his-C/EBPβ-3, LZRS-his-C/EBPβ-3, adeno-C/EBPβ-1, adeno-C/EBPβ-3, parental viral vectors, and sham transduction. Cells of each type and with each transduction regimen are plated at approximately 800 cells per dish. After 7 to ten days in culture, cells are stained with hematoxylin and the number and size of the colonies is determined. It is expected that positively treated cells will proliferate more slowly and form colonies more poorly than parental vector or sham transduced cells.

A colony assay is performed with MDA 231 breast cancer cells transduced with LZRS-his-C/EBPβ-3 (see FIG. 11). Immunofluorescent staining indicated that approximately 80% of the MDA 231 cells are transduced by the LZRS-his-C/EBPβ-3 in this particular experiment (data not shown). After one week in culture, the non-transduced cells readily proliferate and colonies are easily visible upon hematoxylin staining. In contrast, only a few colonies are visible with the LZRS-his-C/EBPβ-3 transduced cells. These data demonstrate that the introduction of exogenous C/EBPβ-3 into tumor cells inhibits growth and/or induces cell death in the MDA 231 cell population. Of further note is that cell proliferation is inhibited or cell death is stimulated in the 20% of tumor derived cells that are not transduced by the LZRS-his-C/EBPβ-3 vector. Given an 80% transduction rate, it is expected that about 200 colonies out of 800 should form; however, fewer than ten colonies are identifiable. This is evidence that the “bystander effect” occurs during the course of treatment by the present invention. The bystander effect is where the biological activity of a treatment introduced into a subset of cells is effective on nearby unmodified cells. The mechanism(s) of the bystander effect are not well understood, but empirically the existence of the bystander effect is well known (see e.g., Gene Therapy of Cancer (1999) Lattime et al., (eds.) Academic Press, especially pp:158-164, 328, incorporated herein by reference).

### EXAMPLE 11

### Growth Potential: Colony Forming Ability in Soft Agar

The ability of cells to form colonies in soft agar has long been used to assess anchorage independence and tumorigenesis and can be used to determine biological activity of a composition of the present invention including biologically function equivalents. Comparisons are made between cells and experimental conditions as in the previous section except that the cells are plated in soft agar. It is expected that HMECs will have a relatively low ability to grow in soft agar compared to the panel of mammary carcinoma cell lines and the cells derived from primary breast tumors. Transduction with LZRS-his-C/EBPβ-1, LZRS-his-C/EBPβ-3, adeno-C/EBPβ-1, and adeno C/EBPβ-3 are all expected to significantly reduce the colony forming potential of each carcinoma cell type compared to the parental vector (without insert), or sham transduction.

### EXAMPLE 12

### Growth Potential: Tumorigenicity in Vivo

The nude mouse, which is immuno-compromised, has long served as a model for the tumorigenic potential of implanted cells. In this series of tests, non-producer C/EBPβ1 and/or C/EBPβ-3 retrovirus or adenovirus transduced cells are harvested, washed with PBS (phosphate buffered saline), and implanted subcutaneous (s.c.) into the mammary pad of female nude mice. Control-transduced or non-transduced cells are also implanted into a separate group of nude mice as controls. If the cell line requires estrogen for growth, the nude mice will also receive a slow release estrogen pellet s.c. 24 hours prior to injection of the cells. Tumor formation is assessed for about 10 weeks or until the tumor size reaches about 5 to 6 mm in diameter. The latency and size of tumor formation in the population of nude mice receiving cells overexpressing C/EBPβ-1 or C/EBPβ-3 will be compared with controls. By performing the tumorigenicity and anchorage-independent growth studies with the panel of mammary carcinoma cell lines, any possible correlation between the ability of C/EBPβ-3 overexpression to suppress tumor formation or anchorage-independent cell growth and overexpression of the EGFR family of tyrosine kinase receptors, estrogen receptor status, or endogenous level of C/EBPβ-3; can be examined. The inhibition of tumorigenicity can be measured in this manner.

### EXAMPLE 13

### Growth Potential: Influence on Gene Expression

The influence of C/EBPβ-1 and C/EBPβ-3 expression on the transcription of some key growth regulatory genes will be determined, using methods known to one with skill in the art. The transcription of additional endogenous genes can be evaluated also by similar methods. The initial battery of genes to be examined include cfos, cmyc, cyclin D1, cyclin E, the CDK inhibitors p16^{INK4} and p27^{KIP1}, and p21^{WAF1/CIP1}. At 24 hour post-transduction and various time intervals thereafter, the endogenous expression of these genes is monitored by Northern blotting or RNase protection. Protein levels are analyzed in parallel by Western blotting.

It is expected that in C/EBPβ-3 overexpressing, growth arrested cells, expression of one or more growth promoting genes, such as cfos, cmyc, cyclin D1 and cyclin E, will be down regulated at either the protein or mRNA level or both. Conversely, whether any genes involved in growth arrest, such as p16^{INK4}, p27^{KIP1}, and p21^{WAF1/CIP1} are concomitantly up regulated will be determined. It is important to note that while these experiments may help to establish a mechanism for the inhibition of growth by C/EBPβ-3 (either direct or indirect), the use of C/EBPβ-1 and/or C/EBPβ-3 as anti-tumor agents is not limited by any particular mechanism.

It is expected that in C/EBPβ-1 overexpressing, growth arrested cells, expression of one or more growth promoting genes, such as cfos, cmyc, cyclin D1 and cyclin E, will be down regulated, also, at either the protein or mRNA level or both. Conversely, whether any genes involved in growth arrest, such as p16^{INK4}, p27^{KIP1}, and p21^{WAF1/CIP1} are concomitantly up regulated will be determined. It is important to note that while these experiments may help to establish a mechanism for the inhibition of growth by C/EBPβ-1 (either direct or indirect), the use of C/EBPβ-1 and/or C/EBPβ-3 as anti-tumor agents is not limited by any particular mechanism.

### EXAMPLE 14

### Growth Potential: Combined Action of C/EBPβ-1 and C/EBPβ-3

The effect of expressing both C/EBPβ-1 and C/EBPβ-3 on cancer cell growth will be examined. In this particular embodiment, C/EBPβ-1 is expressed by transduction with LZRS-his-C/EBPβ-1 and C/EBPβ-3 is expressed by transduction with adeno-C/EBPβ-3. The cells tested include, but are not limited to: HMECs (normal control), the panel of mammary carcinoma cell lines (MDA468, MDA 231, MCF7-neo, MCF7-218, MCF7, T47D, and BT474), and cells derived from primary breast tumors. The cells are first transduced with the LZRS-his-C/EBPβ-1 viral vector followed by the adeno-C/EBPβ-3 viral vector 24 hours later. This procedure is chosen because although retroviruses require the cells to be proliferating for viral integration, adenoviruses do not; quiescent cells are efficiently transduced by adenovirus. Thus, the cells are still efficiently transduced by adeno-C/EBPβ-3 regardless of cell cycle arrest induced by LZRS-his-C/EBPβ-1.

LZRS-his-C/EBPβ-1 and adeno-C/EBPβ-3 carry different epitope tags; so, the fraction of cells expressing each protein is determined by immunofluorescent co-staining. LZRS-his-C/EBPD-1 expresses a histidine (His) tagged C/EBPβ-1 which is detected with FITC-conjugated 6-His mouse monoclonal antibody (Babco). Adeno-C/EBPβ-3 expresses the influenza hemagglutinin (HA) epitope tagged C/EBPβ-3 which is identified using a rabbit anti-HA antibody and rhodamine conjugated anti-rabbit secondary antibody. Co-expressing cells give rise to yellow fluorescence. The growth analyses described above for independent transductions with each viral vector of the present invention are repeated for the combination of C/EBPβ-1 and C/EBPβ-3 co-transduction and co-expression including: S phase distribution, doubling time, colony forming assay, soft agar growth and tumorigenicity in nude mice, and influence on gene expression.

Even though both C/EBPβ-3 and C/EBPβ-1 arrest cancer cell growth, introducing the two isoforms together may be more beneficial for several reasons. First, increased efficacy may be observed with the two proteins together. If a higher proportion of the cells are growth arrested and subsequent cell death ensues more quickly, this has obvious advantages for tumor therapy. Moreover, it is contemplated that certain tumor cells might escape growth arrest by either C/EBPβ-3 and C/EBPβ-1 singly, by overexpressing C/EBPβ-2, or another mechanism. C/EBPβ-2 homodimers could directly compete for promoter sites. C/EBPβ-2 readily heterodimerizes with C/EBPβ-3, although it is not clear whether heterodimerization with C/EBPβ-2 would necessarily neutralize C/EBPβ-3 activity. Whether C/EBPβ-2 heterodimerizes with C/EBPβ-1 in the cell is not known. Nonetheless, expression of both C/EBPβ-1 and C/EBPβ-3 may make escape of the tumor cell from treatment according to the present invention more difficult, regardless of mechanism. Also, by promoting a differentiation pathway or upregulating p21, C/EBPβ-1 is contemplated to act further downstream of cyclin D1 to inhibit growth. Thus, if C/EBPβ-3 proves to act through affecting cyclin D1 status, then C/EBPβ-1 may inhibit the escape of certain tumor cells that are resistant to treatment with C/EBPβ-3. The effectiveness of C/EBPβ-1, C/EBPβ-3, and combined therapy with C/EBPβ-1 and C/EBPβ-3 on the growth and tumorigenicity parameters listed above will be determined for a number of cancer cell types. A correlation between the effectiveness of each therapy with estrogen receptor status, erbB2 receptor status, EGF receptor status, cyclin D1 overexpression, Rb, p16, and p53 status will be made. Although it is possible to transduce cell with both retroviruses and adenoviruses, the inventor contemplates that, in vivo, C/EBPβ-3 and C/EBPβ-1 will be expressed from the same mRNA by use of an internal ribosome entry site (IRES) as is known in the art. Additional studies with many more cell lines and primary tumors, including tumors derived from tissues other than will also be performed. The tests described herein can be used in the determination of biological activity of a particular C/EBPβ-1/C/EBPβ-3 sequence.

### EXAMPLE 15

Administration of isolated C/EBPβ-3 (polypeptide or as a expressed from an encoding nucleic acid) is further tested in the following cells which serve as models for the indicated types of cancers or tumors. The cell lines are available from the American Type Culture Collection (ATCC, Rockville, Md.) and the ATCC number is provided for convenience in ordering. This list is present by way of example and is non-limiting.

| Tumor type | Cell line | Characteristics | ATCC # |
|---|---|---|---|
| Prostate | PC3 | adenocarcinoma, metastatic | CRL-1435 |
| Colon | HCT15 | adenocarcinoma, epithelial | CCL225 |
| Colon | SW48 | adenocarcinoma, epithelial | CCL231 |
| Pancreatic | PANC1 | epitheloid carcinoma of | CRL1469 |
| | | pancreas duct | |
| Liver | HepG2 | hepatocellular carcinoma | HB8065 |
| Stomach | RF-1 | gastric adenocarcinoma | CRL1864 |
| Bladder | HT1376 | carcinoma urinary bladder | CRL1472 |
| Ovarian | ES-2 | carcinoma, ovary | CRL1978 |
| Cervical | Hela | adenocarcinoma, cervix | CCL-2 |
| Endometrium | KLE | adenocarcinoma, utereus | CRL1622 |
| Epidermis | A431 | epidermoid carcinoma | CRL1555 |
| Skin | C32 | melanoma | CRL1585 |
| Bone | SK-ES-1 | Ewing sarcoma, bone | HTB-86 |
| Eye | Y79 | retinoblastoma | HTB-18 |
| Adrenal | SW13 | Small cell carcinoma adrenal | CCL105 |
| | | cortex | |
| Lung | DM5 79 | carcinoma, small cell lung | CRL2049 |

It is contemplated that C/EBPβ-3 (p20) inhibits growth and/or promotes cell death in some or all of the above tumor models. It is also contemplated that C/EBPβ-3 inhibits growth and/or promotes cell death in tumors of epithelial origin. It is further contemplated that C/EBPβ-3 inhibits growth and/or promotes cell death in tumors of mesenchymal origin. It is contemplated that administration of isolated C/EBPβ-3 to any type of cancer/tumor, including those listed in the present example, comprises a treatment of the cancer/tumor, including inhibition of tumorigenesis.

### EXAMPLE 16

Example 15 is repeated except that C/EBPβ-3 is replaced with C/EBPβ-1.

### EXAMPLE 17

Example 15 is repeated except that C/EBPβ-3 is replaced with C/EBPβ-1 and C/EBPβ-3.

### EXAMPLE 18

Transgenic mice are generated using standard methods wherein the mice include an isolated nucleic acid for the expression of C/EBPβ-3 and wherein the mice express the transgenic C/EBPβ-3. The transgenic C/EBPβ-3 mice are crossed with each of the following transgenic mice that overexpress: TGFα, neu, ras, myc, ras-myc bigenic, and TGFα-myc bigenic. The bigenic and trigenic mice expressing C/EBPβ-3 are compared to their respective counterpart to determine the effect of the introduction (administration) of C/EBPβ-3 into each of these models for cancer and tumorigenesis. It is expected that C/EBPβ-3 expressing mice will have fewer tumors and/or tumor formation will be delayed.

### EXAMPLE 19

Example 17 is repeated except that the isolated C/EBPβ-3 expressing nucleic acid is operably linked to an MMTV promoter.

### EXAMPLE 20

Additional experiments will include the transplantation of C/EBPβ-3 expressing MDA 231 cells into nude mice using standard techniques with comparison to non-expressing controls. For example, by infecting MDA 231 cells with LZRS-his-C/EBPβ-3-IRES-eGFP and sorting of C/EBPβ-3 expressing versus non-expressing cells. It is expected that C/EBPβ-3 expression will substantially reduce or even eliminate the transforming or tumorigenic potential of the MDA 231 cells.

### EXAMPLE 21

Additional experiments will include the transplantation of C/EBPβ-1 expressing MDA 231 cells into nude mice with comparison to non-expressing controls. For example, by infecting MDA 231 cells with LZRS-his-C/EBPβ-1-IRES-eGFP and sorting of C/EBPβ-1 expressing versus non-expressing cells. It is expected that C/EBPβ-1 expression will substantially reduce or even eliminate the transforming or tumorigenic potential of the MDA 231 cells.

### EXAMPLE 22

Additional experiments will include the transplantation of C/EBPβ-1 and C/EBPβ-3 expressing MDA 231 cells into nude mice with comparison to non-expressing controls. For example, by infecting MDA 231 cells with an LZRS-his-C/EBPβ-1-IRES-C/EBPβ-3-IRES-eGFP and sorting of C/EBPβ-1 expressing versus non-expressing cells. It is expected that C/EBPβ-1 expression will substantially reduce or even eliminate the transforming or tumorigenic potential of the MDA 231 cells.

What is claimed is:

1. A method of treating a tumor in a mammal in need thereof, comprising administering an anti-tumor effective amount of an isolated C/EBPβ isoform to the mammal.

2. The method of claim 1, wherein the isolated C/EBPβ isoform comprises an isolated C/EBPβ-1 isoform polypeptide.

3. The method of claim 1, wherein the isolated C/EBPβ isoform comprises an isolated C/EBPβ-3 isoform polypeptide.

4. The method of claim 1, wherein the isolated C/EBPβ isoform comprises an isolated C/EBPβ-1 isoform polypeptide and an isolated C/EBPβ-3 isoform polypeptide.

5. The method of claim 1, wherein the isolated C/EBPβ isoform is substantially free of a C/EBPβ-2 isoform.

6. The method of claim 1, wherein the administering step further comprises introducing a pharmaceutical formulation to the mammal, wherein the pharmaceutical formulation includes the C/EBPβ isoform and a pharmaceutically acceptable carrier.

7. The method of claim 6, wherein the pharmaceutical formulation is substantially free of a C/EBPβ-2 isoform.

8. The method of claim 6, wherein the pharmaceutically acceptable carrier comprises a liposome.

9. The method of claim 1, wherein the administering step includes introducing the isolated C/EBPβ isoform ex vivo to a mammal compatible carrier and introducing the mammal compatible carrier to the mammal.

10. The method of claim 1, wherein the tumor is a breast tumor.

11. The method of claim 1, wherein the tumor is a metastatic tumor.

12. The method of claim 1, wherein the tumor is selected from the following group: an epithelial tumor, a mesenchymal tumor, a prostate tumor, a colorectal tumor, a pancreatic tumor, a liver tumor, a stomach tumor, a bladder tumor, an ovarian tumor, a cervical tumor, a tumor of the endometrium, a tumor of the epidermis, a skin tumor, a bone tumor, a tumor of the eye, an adrenal tumor, and a lung tumor.

13. The method of claim 1, wherein the administering step includes introducing the C/EBPβ isoform to a cell of the mammal, wherein the cell is selected from a group consisting of: an endothelial cell, a vascular endothelial cell, an epithelial cell, a mammary epithelial cell, a lymphoid cell, and a myeloid cell.

14. The method of claim 13, wherein the isolated C/EBPβ isoform includes the polypeptide sequence listed in SEQ ID NO:5 and conservatively modified variants thereof or the amino acid sequence listed in SEQ ID NO:7 and conservatively modified variants thereof.

15. The method of claim 13, wherein the administrating step further comprises introducing a fusion protein to the mammal, wherein fusion protein includes the C/EBPβ isoform and a membrane transport sequence operatively linked to the C/EBPβ isoform.

16. The method of claim 1, wherein the administering step includes administering a nucleic acid to a cell of the mammal and expressing a C/EBPβ isoform polypeptide in the cell, wherein the nucleic acid comprises a polynucleotide segment encoding the C/EBPβ isoform polypeptide.

17. The method of claim 16, wherein the polynucleotide segment encodes a C/EBPβ-1 isoform polypeptide, a C/EBPβ-3 isoform polypeptide, or both the C/EBPβ-1 isoform polypeptide and the C/EBPβ-3 isoform polypeptide.

18. The method of claim 17, wherein the nucleic acid comprises an expression vector operatively linked to the polynucleotide segment and having at least one control element for expressing the C/EBPβ isoform polypeptide in the cell.

19. The method of claim 17, further comprising modifying the polynucleotide segment to prevent a production of a C/EBPβ-2 isoform polypeptide.

20. The method of claim 16, wherein the polynucleotide segment includes an isolated polynucleotide sequence as set forth in SEQ ID NO:17 and conservatively modified variants thereof or an isolated poly-nucleic acid sequence as set forth in SEQ ID NO:4 and conservatively modified variants thereof.

21. The method of claim 16, wherein the nucleic acid comprises a viral expression vector operatively linked to the polynucleotide segment and having at least one genetic control element for expressing the C/EBPβ isoform polypeptide in the cell.

22. The method of claim 21, wherein the viral expression vector is an adenovirus expression vector, a retroviral expression vector, or a hybrid retrovirus-Epstein Barr virus vector.

23. A kit for treating a tumor in a mammal in need thereof, comprising one or more suitable containers holding an amount of an isolated C/EBPβ isoform polypeptide and a set of instructions for administering the isolated C/EBPβ isoform to the mammal, wherein the isolated C/EBPβ isoform polypeptide is an isolated C/EBPβ-1 isoform polypeptide or a C/EBP3 isoform polypeptide, and wherein the isolated C/EBPβ isoform polypeptide is substantially free of a C/EBPβ-2 isoform polypeptide.

24. A kit for treating a tumor in a mammal in need thereof, comprising one or more suitable containers holding an amount of an isolated expression vector including an expression insert having a polynucleotide segment encoding a C/EBPβ isoform, wherein the expression vector includes at least one genetic control element operatively linked to the expression insert for driving an expression of the C/EBPβ isoform in a cell of the mammal.

25. The kit of claim 24, further comprising a set of instructions for administering the isolated expression vector to the mammal.

26. The kit of claim 24, wherein the insert is modified to prevent a production of a C/EBPβ-2 isoform.

27. A method of inhibiting tumorigenesis in a population of cells in a mammal comprising administering an effective amount of an isolated C/EBPβ isoform to the population of cells in the mammal, wherein the isolated C/EBPβ isoform is an isolated C/EBPβ-1 isoform or an isolated C/EBPβ-3 isoform.

28. A method of inhibiting proliferation of a mammalian cell, comprising administering an effective amount of an isolated C/EBPβ isoform to the mammalian cell, wherein the isolated C/EBPβ isoform is an isolated C/EBPβ-1 isoform or an isolated C/EBPβ-3 isoform.

29. A method of inducing differentiation of a mammalian cell, comprising introducing an effective amount of an isolated C/EBPβ-1 isoform to the mammalian cell.

30. A method of promoting a cell death of a mammalian cell, comprising administering an effective amount of an isolated C/EBPβ-3 isoform to the mammalian cell.

31. A composition comprising an isolated polynucleotide encoding a C/EBPβ-1 isoform having a mutation which eliminates a C/EBPβ-2 isoform production site.

32. The composition of claim 31, wherein the C/EBPβ-2 isoform production site comprises a C/EBPβ-2 translation start site.

33. The composition of claim 32, wherein the polynucleotide further comprises a Kozak site at a C/EBPβ-1 translation initiation site.

34. The composition of claim 32, further comprising an expression vector operatively linked to the C/EBPβ-1 polynucleotide sequence for driving expression of a C/EBPβ-1 isoform polypeptide in a mammalian cell.

35. The composition of claim 34, further comprising an internal ribosomal entry site and a second C/EBPβ-3 polynucleotide sequence.

36. The composition of claim 35, further comprising at least one membrane transport sequence.

37. A composition comprising an isolated polypeptide including a membrane transport sequence operably linked to a C/EBPβ-1 isoform polypeptide or a C/EBPβ-3 isoform polypeptide.

38. A method of manufacturing an anti-tumor agent comprising the steps of:
a. obtaining a polynucleotide sequence encoding a C/EBPβ-1 isoform of a C/EBPβ;
b. modifying the polynucleotide sequence encoding the C/EBPβ-1 isoform to prevent a production of a C/EBPβ-2 isoform;
c. combining the modified polynucleotide sequence encoding the C/EBPβ-1 isoform with a nucleic acid capable of expressing a C/EBPβ-1 isoform polypeptide in a cell; and
d. expressing the C/EBPβ-1 isoform polypeptide in the cell.

39. The method of claim 38, further comprising isolating an essentially pure C/EBPβ-1 isoform polypeptide.

40. The method of claim 38, wherein the modifying step comprises mutating a translation initiation site for the C/EBPβ-2 isoform.

41. A method of making an anti-tumor agent comprising the steps of:
a. obtaining a polynucleotide sequence encoding a C/EBPβ-3 isoform of a C/EBPβ;
b. combining the modified polynucleotide sequence encoding the C/EBPβ-1 isoform with a nucleic acid capable of expressing a C/EBPβ-1 isoform polypeptide in a cell; and
c. expressing the C/EBPβ-1 isoform polypeptide in the cell.
